(19) United States
(12) Patent Application Publication
(10) Pub. No.: US 2024/0285801 A1
(43) Pub. Date: Aug. 29, 2024

(54) METHODS AND MATERIALS FOR TREATING MUSCULAR DYSTROPHY

(71) Applicant: UNIVERSITY OF PITTSBURGH - OF THE COMMONWEALTH SYSTEM OF HIGHER EDUCATION, Pittsburgh, PA (US)

(72) Inventors: Dwi U. Kemaladewi, Pittsburgh, PA (US); Robert David Nicholls, Pittsburgh, PA (US); Jia Qi Cheng Zhang, Pittsburgh, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 18/588,657

(22) Filed: Feb. 27, 2024

Related U.S. Application Data
(60) Provisional application No. 63/529,236, filed on Jul. 27, 2023.
(60) Provisional application No. 63/448,599, filed on Feb. 27, 2023.

(51) Int. Cl. A61K 48/00; A61K 38/39; A61P 21/00; C12N 9/22; C12N 15/11; C12N 15/86

(52) U.S. Cl. CPC A61K 48/005; A61K 38/39; A61P 21/00; C12N 9/22; C12N 15/11; C12N 15/86; C12N 2310/20; C12N 2750/14143

(57) ABSTRACT

This document provides methods and materials for treating a mammal (e.g., a human) having, or at risk of developing, muscular dystrophy (e.g., a laminin α2-related dystrophy (LAMA2-RD)). For example, an adeno-associated virus (AAV) designed for targeted gene activation of nucleic acid encoding a Lama1 polypeptide (e.g., the endogenous Lama1 gene) can be administered to a mammal (e.g., a human) having, or at risk of developing, muscular dystrophy (e.g., a LAMA2-RD) to treat the mammal.

### CROSS-REFERENCE TO RELATED APPLICATIONS

This application claims the benefit of U.S. Patent Application Ser. No. 63/448,599, filed on Feb. 27, 2023, and U.S. Patent Application Ser. No. 63/529,236, filed on Jul. 27, 2023. The disclosures of the prior applications are considered part of, and are incorporated by reference in, the disclosure of this application.

### STATEMENT REGARDING FEDERAL FUNDING

This invention was made with government support under AR078872 awarded by the National Institutes of Health. The government has certain rights in the invention.

### SEQUENCE LISTING

This application contains a Sequence Listing that has been submitted electronically as an XML file named “48881-0049001_SL.xml.” The XML file, created on Feb. 13, 2024, is 94000 bytes in size. The material in the XML file is hereby incorporated by reference in its entirety.

### TECHNICAL FIELD

This document relates to methods and materials for treating a mammal (e.g., a human) having, or at risk of developing, muscular dystrophy (e.g., a laminin α2-related dystrophy (LAMA2-RD)). For example, adeno-associated virus (AAV) vectors designed for targeted gene activation of nucleic acid encoding a Lama1 polypeptide (e.g., the endogenous Lama1 gene) can be administered to a mammal (e.g., a human) having, or at risk of developing, muscular dystrophy (e.g., a LAMA2-RD) to treat the mammal.

### BACKGROUND

Congenital muscular dystrophies (CMDs) are a heterogeneous set of neuromuscular diseases with varying clinical phenotypes and genetic causes. CMDs are mapped across 30 genetic loci and characterized by their disease onset at birth or in infancy (Gawlik et al., Int. J. Mol. Sci., 19(5):1490 (2018)). The prevalence of CMD is estimated to range from 0.68 to 2.5 per 100,000 (Norwood et al., Brain, 132:3175-3186 (2009)). LAMA2-RD subtypes are caused by mutations in the LAMA2 gene, which encodes a laminin-α2 polypeptide, and make up about 30% to 40% of total CMD cases. LAMA2-RD leads to destabilization of the basement membrane, which causes skeletal muscle damage and abnormal nerve myelination. There is currently no cure available for any form of CMD.

### SUMMARY

This document relates to methods and materials for treating a mammal (e.g., a human) having, or at risk of developing, muscular dystrophy (e.g., a LAMA2-RD). In some cases, this document provides AAV vectors designed for targeted gene activation of nucleic acid encoding a Lama1 polypeptide (e.g., the endogenous Lama1 gene). For example, AAV vectors designed for targeted gene activation of nucleic acid encoding a Lama1 polypeptide (e.g., the endogenous Lama1 gene) can be administered to a mammal (e.g., a human) having, or at risk of developing, muscular dystrophy (e.g., a LAMA2-RD) to treat the mammal. For example, AAV vectors designed for targeted gene activation of nucleic acid encoding a Lama1 polypeptide (e.g., the endogenous Lama1 gene) can be used to increase expression of a Lama1 polypeptide by cells within a mammal (e.g., a human) having, or at risk of developing, muscular dystrophy (e.g., a LAMA2-RD) to treat the mammal.

Upregulation of the compensatory gene Lama1 (e.g., resulting in expression of a Lama1 polypeptide) can be used to treat LAMA2-RDs. As demonstrated herein, AAV vectors designed for targeted gene activation of an endogenous nucleic acid encoding a Lama1 polypeptide (e.g., an endogenous Lama1 gene) can be used to increase expression of a Lama1 polypeptide within a cell (e.g., within a cell within a mammal such as a human). For example, an AAV vector having a genome (e.g., a single-stranded or double-stranded DNA genome) including a nucleic acid sequence (e.g., an engineered DNA sequence) including (a) a nucleic acid encoding a guide RNA (gRNA) that is complementary to a target site upstream of a transcriptional start site of an endogenous nucleic acid sequence encoding a Lama1 polypeptide (e.g., an endogenous Lama1 gene), and (b) a nucleic acid encoding a catalytically inactive Cas (dCas) polypeptide including one or more transcriptional activators can use CRISPR activation (CRISPRa) to increase expression of a Lama1 polypeptide within a cell (e.g., within a cell within a mammal such as a human).

Having the ability to increase expression of a Lama1 polypeptide within a cell (e.g., within a cell within a mammal such as a human) using one or more AAV vectors provided herein (e.g., one or more AAV vectors designed for targeted gene activation of nucleic acid encoding a Lama1 polypeptide) provides a unique and unrealized opportunity to treat LAMA2-RDs. In addition, AAV vectors provided herein (e.g., one or more AAV vectors designed for targeted gene activation of nucleic acid encoding a Lama1 polypeptide) can be produced using high throughput and cost-effective methods. In some cases, a single AAV vector provided herein can be used to target gene activation of nucleic acid encoding an endogenous Lama1 polypeptide to treat LAMA2-RDs.

In general, one aspect of this document features adeno-associated viruses comprising a single-stranded DNA, where the single-stranded DNA comprises a first inverted terminal repeat (ITR) sequence followed by an engineered DNA sequence followed by a second ITR sequence, where the engineered DNA sequence comprises (a) a first DNA sequence comprising a first promotor sequence operably linked to a DNA sequence encoding a guide RNA or (a-ii) a reverse-complement thereof and (b) a second DNA sequence comprising (b-i) a second promotor sequence operably linked to a DNA sequence encoding a catalytically inactive Cas polypeptide, where the guide RNA is at least 95 percent complementary to a genomic sequence located between 0 and 1000 nucleotides upstream of a transcriptional start site of an endogenous nucleic acid sequence encoding a Lama1 polypeptide within a mammal, where the second promotor sequence comprises at least four motifs having the DNA sequence set forth in SEQ ID NO:45 (5′-YGCGCANGCGCR-3′), where 8 to 12 intervening nucleotides are located between each adjacent motif of the at least four motifs, where the catalytically inactive Cas polypeptide comprises at least one transcriptional activator, and where delivery of the adeno-associated virus to a cell within the mammal results in expression of the Lama1 polypeptide within the cell. The mammal can be a human. The cell can be a muscle cell. The cell can be a skeletal muscle cell. The cell can be a Schwann cell. The adeno-associated virus can be AAV6, AAV8, AAV2, or AAV9. The first ITR sequence can be directly followed by the engineered DNA sequence. The first ITR sequence can be indirectly followed by the engineered DNA sequence. The engineered DNA sequence can be directly followed by the second ITR sequence. The engineered DNA sequence can be indirectly followed by the second ITR sequence. The first ITR sequence can include the DNA sequence set forth in SEQ ID NO:67. The second ITR sequence can include the DNA sequence set forth in SEQ ID NO: 69. The first promotor sequence can be a type 3 RNA polymerase III promoter. The first promotor sequence can be a 7SK promoter, a H1 promoter, or a U6 promotor. The guide RNA can be complementary to the genomic sequence. The genomic sequence can be located between 0 and 500 nucleotides upstream of the transcriptional start site. The guide RNA can include the RNA sequence set forth in SEQ ID NO:1. The second promotor sequence can include four of the motifs. Each of the motifs can include a DNA sequence set forth in any one of SEQ ID NOs: 46 to 55. Each of the motifs can include a DNA sequence set forth in any one of SEQ ID NOs: 46 to 49. Each of the 8 to 12 intervening nucleotides can include a DNA sequence set forth in any one of SEQ ID NOs: 56 to 64. Each of the 8 to 12 intervening nucleotides can include a DNA sequence set forth in any one of SEQ ID NOs: 56 to 58. The catalytically inactive Cas polypeptide can be a catalytically inactive Cas9 polypeptide. The catalytically inactive Cas polypeptide can be a Staphylococcus aureus deactivated Cas9 (SadCas9) polypeptide. The catalytically inactive Cas polypeptide can include the amino acid sequence set forth in SEQ ID NO:43. The catalytically inactive Cas polypeptide can include a VP64 transcriptional activator. The catalytically inactive Cas polypeptide can include a VP64-Δp65-ΔRTA transcriptional activator. The VP64-Δp65-ΔRTA transcriptional activator can include the amino acid sequence set forth in SEQ ID NO:44.

In another aspect, this document features isolated nucleic acid molecules including an engineered DNA sequence having (a) a first DNA sequence comprising a first promotor sequence operably linked to a DNA sequence encoding a guide RNA or (a-ii) a reverse-complement thereof and (b) a second DNA sequence comprising (b-i) a second promotor sequence operably linked to a DNA sequence encoding a catalytically inactive Cas polypeptide, where the guide RNA is at least 95 percent complementary to a genomic sequence located between 0 and 1000 nucleotides upstream of a transcriptional start site of an endogenous nucleic acid sequence encoding a Lama1 polypeptide within a mammal, where the second promotor sequence comprises at least four motifs having the DNA sequence set forth in SEQ ID NO:45 (5′-YGCGCANGCGCR-3′), where 8 to 12 intervening nucleotides are located between each adjacent motif of the at least four motifs, where the catalytically inactive Cas polypeptide comprises at least one transcriptional activator.

In another aspect, this document features in vitro host cells, where the host cell comprises an adeno-associated virus comprising a single-stranded DNA, where the single-stranded DNA comprises a first ITR sequence followed by an engineered DNA sequence followed by a second ITR sequence, where the engineered DNA sequence comprises (a) a first DNA sequence comprising a first promotor sequence operably linked to a DNA sequence encoding a guide RNA or (a-ii) a reverse-complement thereof and (b) a second DNA sequence comprising (b-i) a second promotor sequence operably linked to a DNA sequence encoding a catalytically inactive Cas polypeptide, where the guide RNA is at least 95 percent complementary to a genomic sequence located between 0 and 1000 nucleotides upstream of a transcriptional start site of an endogenous nucleic acid sequence encoding a Lama1 polypeptide within a mammal, where the second promotor sequence comprises at least four motifs having the DNA sequence set forth in SEQ ID NO:45 (5′-YGCGCANGCGCR-3′), where 8 to 12 intervening nucleotides are located between each adjacent motif of the at least four motifs, where the catalytically inactive Cas polypeptide comprises at least one transcriptional activator, and where delivery of the adeno-associated virus to a cell within the mammal results in expression of the Lama1 polypeptide within the cell. The host cell can be a muscle cell, a fibroblast, a Schwann cell, or an epithelial cell.

In another aspect, this document features in vitro host cells, where the host cell comprises a nucleic acid molecule including an engineered DNA sequence having (a) a first DNA sequence comprising a first promotor sequence operably linked to a DNA sequence encoding a guide RNA or (a-ii) a reverse-complement thereof and (b) a second DNA sequence comprising (b-i) a second promotor sequence operably linked to a DNA sequence encoding a catalytically inactive Cas polypeptide, where the guide RNA is at least 95 percent complementary to a genomic sequence located between 0 and 1000 nucleotides upstream of a transcriptional start site of an endogenous nucleic acid sequence encoding a Lama1 polypeptide within a mammal, where the second promotor sequence comprises at least four motifs having the DNA sequence set forth in SEQ ID NO:45 (5′-YGCGCANGCGCR-3′), where 8 to 12 intervening nucleotides are located between each adjacent motif of the at least four motifs, where the catalytically inactive Cas polypeptide comprises at least one transcriptional activator. The host cell can be a muscle cell, a fibroblast, a Schwann cell, or an epithelial cell.

In another aspect, this document features compositions comprising an adeno-associated virus comprising a single-stranded DNA, where the single-stranded DNA comprises a first ITR sequence followed by an engineered DNA sequence followed by a second ITR sequence, where the engineered DNA sequence comprises (a) a first DNA sequence comprising a first promotor sequence operably linked to a DNA sequence encoding a guide RNA or (a-ii) a reverse-complement thereof and (b) a second DNA sequence comprising (b-i) a second promotor sequence operably linked to a DNA sequence encoding a catalytically inactive Cas polypeptide, where the guide RNA is at least 95 percent complementary to a genomic sequence located between 0 and 1000 nucleotides upstream of a transcriptional start site of an endogenous nucleic acid sequence encoding a Lama1 polypeptide within a mammal, where the second promotor sequence comprises at least four motifs having the DNA sequence set forth in SEQ ID NO:45 (5′-YGCGCANGCGCR-3′), where 8 to 12 intervening nucleotides are located between each adjacent motif of the at least four motifs, where the catalytically inactive Cas polypeptide comprises at least one transcriptional activator, and where delivery of the adeno-associated virus to a cell within the mammal results in expression of the Lama1 polypeptide within the cell.

In another aspect, this document features methods for increasing expression of a Lama1 polypeptide by cells within a mammal. The methods can include, or consist essentially of, administering an adeno-associated virus comprising a single-stranded DNA, where the single-stranded DNA comprises a first ITR sequence followed by an engineered DNA sequence followed by a second ITR sequence, where the engineered DNA sequence comprises (a) a first DNA sequence comprising a first promotor sequence operably linked to a DNA sequence encoding a guide RNA or (a-ii) a reverse-complement thereof and (b) a second DNA sequence comprising (b-i) a second promotor sequence operably linked to a DNA sequence encoding a catalytically inactive Cas polypeptide, where the guide RNA is at least 95 percent complementary to a genomic sequence located between 0 and 1000 nucleotides upstream of a transcriptional start site of an endogenous nucleic acid sequence encoding a Lama1 polypeptide within a mammal, where the second promotor sequence comprises at least four motifs having the DNA sequence set forth in SEQ ID NO:45 (5′-YGCGCANGCGCR-3′), where 8 to 12 intervening nucleotides are located between each adjacent motif of the at least four motifs, where the catalytically inactive Cas polypeptide comprises at least one transcriptional activator, and where delivery of the adeno-associated virus to a cell within the mammal results in expression of the Lama1 polypeptide within the cell to a mammal, thereby increasing expression of the Lama1 polypeptide by the cells within the mammal. The mammal can be a human.

In another aspect, this document features methods for treating muscular dystrophy. The methods can include, or consist essentially of, administering an adeno-associated virus comprising a single-stranded DNA, where the single-stranded DNA comprises a first ITR sequence followed by an engineered DNA sequence followed by a second ITR sequence, where the engineered DNA sequence comprises (a) a first DNA sequence comprising a first promotor sequence operably linked to a DNA sequence encoding a guide RNA or (a-ii) a reverse-complement thereof and (b) a second DNA sequence comprising (b-i) a second promotor sequence operably linked to a DNA sequence encoding a catalytically inactive Cas polypeptide, where the guide RNA is at least 95 percent complementary to a genomic sequence located between 0 and 1000 nucleotides upstream of a transcriptional start site of an endogenous nucleic acid sequence encoding a Lama1 polypeptide within a mammal, where the second promotor sequence comprises at least four motifs having the DNA sequence set forth in SEQ ID NO:45 (5′-YGCGCANGCGCR-3′), where 8 to 12 intervening nucleotides are located between each adjacent motif of the at least four motifs, where the catalytically inactive Cas polypeptide comprises at least one transcriptional activator, and where delivery of the adeno-associated virus to a cell within a mammal having muscular dystrophy results in expression of the Lama1 polypeptide within the cell, where expression of a Lama1 polypeptide by cells within the mammal is increased following the administering step, and where severity of a symptom of the muscular dystrophy is reduced following the administering step. The mammal can be a human.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTIONS OF THE DRAWINGS

FIGS. 1A-1C. Cell specificity and upregulation strength comparisons between CRISPRa 1.0 and 2.0. FIG. 1A) Schematics of exemplary CRISPRa 1.0 and 2.0 systems. FIG. 1B) 4×NRF1 and CMV driven mNeongreen expression in cell lines of interest. C2C12 mouse myoblasts, LAMA2-RD patient-derived fibroblasts, HEI193 schwannoma cells, and immortalized dy^{2j}/dy^{2j} myoblasts were transfected with plasmids containing the indicated promoters (4×NRF1 or CMV) to drive mNeongreen expression. Cells were imaged 48 hours post-transfection. Scale bars=320 nm. FIG. 1C) tdTomato fluorescent reporter assay carried out in HEK293T cells to compare the upregulation power between CRISPRa 1.0 and 2.0. HEK293T cells were transfected with the reporter plasmid (miniCMV-tdTomato) only, or in combination with the indicated activators. Cells only express tdTomato in the presence of an activator. The cells were imaged at 72 hours post-transfection and analyzed for tdTomato intensity by flow cytometry (p.value<0.0001). Three biological replicates per group were used. Parallel lines represent the group means and significance was determined via the student T-test.

FIGS. 2A-2F. gRNA selection and in vitro validation. FIG. 2A) Scheme representing the upstream region of a Lama1 transcription start site (SEQ ID NO:81) with the selected gRNAs. FIG. 2B) RNA was harvested from C2C12 cells 72 hours post-electroporation with either CRISPRa 2.0 with single gRNA or CRISPRa 1.0 with the three gRNAs. RT-PCR was carried out to detect Lama1 upregulation and the presence of CRISPRa. FIGS. 2C-2F) Upregulation of Lama1 in disease relevant immortalized dy^{2j}/dy^{2j} myoblasts to compare CRISPRa 1.0 with three gRNAs and 2.0 with gRNA1 upregulation strength. FIG. 2C) RT-PCR with three biological replicates. FIG. 2D) qPCR to calculate the fold expression of Lama1 normalized by the housekeeping gene. No significant differences were observed at the transcriptional level. FIG. 2E) Western blot with three biological replicates. FIG. 2F) Western blot densitometry analysis shows a significantly higher 1.88-fold Lama1 upregulation under CRISPRa 2.0 (p. value=0.0177).

FIGS. 3A-3C. Transcriptomic analysis of immortalized dy^{2j}/dy^{2j} myoblasts with CRISPRa 2.0 Lama1 upregulation. FIG. 3A) Volcano plot showing the expression of Lama1. FIG. 3B) Differentially expressed genes (DEG) identified using Qiagen CLG and IPA software with FDR<=0.05. FIG. 3C) Gene enrichment of up and down regulated genes of the differentially expressed genes.

FIGS. 4A-4B. CRISPRa 1.0 and 2.0 were packaged into AAV9 vectors and delivered via intramuscular injection. FIG. 4A) 5-7 weeks old wild-type and LAMA2-RD model mice (dy^{w}/dy^{w} homozygous mice) were injected with CRISPRa 1.0 or 2.0 with and without gRNA(s) as shown in the schematic. 7.5E+11 genome copies (GC) AAV9 were injected per condition, except for the CRISPRa 1.0 with 3gRNAs, which consisted of total 1.5E+12 GC due to the dual AAV9 system. Tibialis anterior were isolated 2 weeks post injection and analyzed for Lama1 polypeptide levels by western blot and immunofluorescent staining. FIG. 4B) Western blot and immunofluorescence images comparing CRISPRa 1.0 and 2.0 for both wild-type and dy^{w}/dy^{w} homozygous mice. Scale bars=60 μm.

FIG. 5. CRISPRa 2.0 gRNA1 AAV9 titration in dy^{w}/dy^{w} homozygous mice. 6 different viral loads of CRISPRa 2.0 gRNA1 AAV9 were delivered intramuscularly, 1.5E+12 GC, 7.5E+11 GC, 3E+11 GC, 2E+11 GC, 1E+11 GC, and 1E+10 GC. Stronger Lama1 upregulation was observed with CRISPRa 2.0 even with a 15 times lower dosage (1E+11 GC).

FIGS. 6A-6C. Nucleotide sequences of an exemplary endogenous mouse Lama1 promoter sequence (FIG. 6A (SEQ ID NO:82)) and exemplary endogenous human LAMA1 promoter sequences (FIG. 6B (SEQ ID NO:83) and FIG. 6C (SEQ ID NO:84)) and the binding locations of exemplary gRNAs.

FIGS. 7A-7B. Systemic administration of CRISPRa 2.0. FIG. 7A) Schematic of systemic administration of AAV9 carrying either CRISPRa 2.0 without gRNA (denoted as CRISPRa 2.0) or with gRNA1 Lama1 (denoted as CRISPRa 2.0 Lama1) at the dose of 7.5E+11 GC per injection. Functional tests and tissue analysis were performed at the indicated time points. FIG. 7B) Western blot of tibialis anterior muscles from wild-type and dy^{w}/dy^{w} mice at 5 weeks post-injection and 11 weeks post-injection.

FIG. 8. LAMA1 expression and histopathology in skeletal muscles following systemic administration of AAV9. Tibialis anterior muscle isolated at 5 weeks post-injection and 11 weeks post-injection were sectioned at 8 μm and subjected to LAMA1 immunofluorescence (scale bars=100 μm) and hematoxylin and eosin (scale bars=210 μm) staining.

FIG. 9. Sciatic nerves were isolated from the mice at the indicated time points, sectioned at 8 μm, and stained for LAMA1, phosphorylated neurofilament H (indicating the axonal fibers), and DAPI (indicating nuclei). LAMA1 was only detected when gRNA1 was used (scale bars=60 μm).

FIG. 10. Sciatic nerves were sectioned at 300 nm for toluidine blue staining (scale bars=50 μm) and sectioned at 60 nm for transmission electron microscopy (scale bars=2 μm). dy^{w}/dy^{w} (denoted as dyw) nerves treated with Lama1 showed decreased demyelinated spots and increased thickness of myelination.

FIG. 11. RT-PCR of Lama1, Sa dCas9, and Gapdh from tissues of interest. The presence of CRISPRa 2.0 is detected in all tissues analyzed but at varying levels.

FIGS. 12A-12I. Functional analyses after systemic administration of CRISPRa 2.0. FIG. 12A) Survival curve of dy^{w}/dy^{w} (denoted as dyw) with CRISPRa 2.0 gRNA1 Lama1 (n=4) and dy^{w}/dy^{w} with CRISPRa 2.0 no gRNA (n=4). FIG. 12B) Body weight from 4 weeks to 11 weeks of dy^{w}/dy^{w} non-injected (n=6), dy^{w}/dy^{w} with CRISPRa 2.0 gRNA1 Lama1 (n=4), and wild-type with CRISPRa 2.0 gRNA1 Lama1 (n=7). FIG. 12C) Representative image of 5-week-old dy^{w}/dy^{w} non-injected, dy^{w}/dy^{w} with CRISPRa 2.0 no gRNA, dy^{w}/dy^{w} with CRISPRa 2.0 gRNA1 Lama1, and wild-type control. FIG. 12D) Body weight of 5-week-old mice. dy^{w}/dy^{w} non-injected (n=20), dy^{w}/dy^{w} with CRISPRa 2.0 no gRNA (n=5), dy^{w}/dy^{w} with CRISPRa 2.0 gRNA1 Lama1 (n=8), wild-type non-injected (n=6), wild-type with CRISPRa 2.0 no gRNA (n=15), wild-type with CRISPRa 2.0 gRNA1 Lama1 (n=10). FIG. 12E) Gastrocnemius weight harvested from 5-week-old mice. dy^{w}/dy^{w} non-injected (n=7), dy^{w}/dy^{w} with CRISPRa 2.0 no gRNA (n=4), dy^{w}/dy^{w} with CRISPRa 2.0 gRNA1 Lama1 (n=4), wild-type non-injected (n=4), wild-type with CRISPRa 2.0 no gRNA (n=5), wild-type with CRISPRa 2.0 gRNA1 Lama1 (n=4). FIG. 12F) Distribution of tibialis anterior muscle fiber diameters, Ferret's diameter of 300 fibers were analyzed per sample. dy^{w}/dy^{w} non-injected (n=6), dyw with CRISPRa 2.0 no gRNA (n=3), dy^{w}/dy^{w} with CRISPRa 2.0 gRNA1 Lama1 (n=3), wild-type non-injected (n=4). Error bars correspond to the SEM. FIG. 12G) Grip strength quantification of 5-week-old mice. dy^{w}/dy^{w} non-injected (n=10), dy^{w}/dy^{w} with CRISPRa 2.0 no gRNA (n=5), dy^{w}/dy^{w} with CRISPRa 2.0 gRNA1 Lama1 (n=8), wild-type non-injected (n=10), wild-type with CRISPRa 2.0 no gRNA (n=9), wild-type with CRISPRa 2.0 gRNA1 Lama1 (n=19). FIG. 12H) Maximum tetanic force normalized to tibia length. dy^{w}/dy^{w} non-injected (n=5), dy^{w}/dy^{w} with CRISPRa 2.0 no gRNA (n=4), dy^{w}/dy^{w} with CRISPRa 2.0 gRNA1 Lama1 (n=4), wild-type non-injected (n=4), wild-type with CRISPRa 2.0 no gRNA (n=5), wild-type with CRISPRa 2.0 gRNA1 Lama (n=4). Note that the tetanic force of dy^{w}/dy^{w} cannot be compared with the wild-type data due to limitations in the Aurora 1300A, in which wild-type gastrocnemius tetanic force exceed the detection range of the 1N sensor. FIG. 12I) Open field rearing activity of 5-week-old mice. dy^{w}/dy^{w} non-injected (n=6), dy^{w}/dy^{w} with CRISPRa 2.0 no gRNA (n=5), dy^{w}/dy^{w} with CRISPRa 2.0 gRNA1 Lama1 (n=8), wild-type non-injected (n=4), wild-type with CRISPRa 2.0 no gRNA (n=15), wild-type with CRISPRa 2.0 gRNA1 Lama1 (n=13).

FIG. 13. Schematic overview of the delayed treatment study. 1 week old mice (dyw and WT) were injected with AAV9 CRISPRa 2.0 (or AAV9 control) via retroorbital administration route at the dose of 7.5×10¹¹ vg/mouse. Open field (OF) and grip strength (GS) tests were performed at the age of 7 weeks old. The animals were monitored for survival.

FIG. 14. AAV9-CRISPRa 2.0 increased survival of the dy^{w}/dy^{w} mice. The lifespan of dy^{w}/dy^{w} mice receiving either control AAV (n=12) or AAV CRISPRa 2.0 (n=8) were monitored. Statistical analysis was performed using Gehan-Breslow Wilcoxon test and p value is indicated.

FIG. 15. AAV9-CRISPRa 2.0 treatment increased body weight of the dy^{w}/dy^{w} mice. The bodyweight of WT and dy^{w}/dy^{w} mice receiving either control AAV or AAV CRISPRa 2.0 were monitored weekly. Statistical analysis was performed using Student's t-test. ***P<0.005. ****P<0.001.

FIG. 16. dy^{w}/dy^{w} grip strength did not change upon AAV9-CRISPRa 2.0 treatment. WT and dy^{w}/dy^{w} mice receiving either control AAV or AAV CRISPRa 2.0 were subjected for grip strength test at the age of 7 weeks old. Statistical analysis was performed using Student's t-test. ns, not significant. **P<0.01, ***P<0.005, ***P<0.001.

FIG. 17. The dy^{w}/dy^{w} mice receiving AAV9-CRISPRa 2.0 demonstrated similar motor performance to wildtype in the open field assay. WT and dy^{w}/dy^{w} mice receiving either control AAV or AAV CRISPRa 2.0 were subjected for 20-minutes open field assay at the age of 7 weeks old. Parameters such as activity, total distance travelled, number of rearing, stereotypy, locomotion, and resting time throughout the 20 minutes test period were captured and compared. Statistical analysis was performed using Student's t-test. ns, not significant. *P<0.05, **P<0.01, ***P<0.005. ****P<0.001.

### DETAILED DESCRIPTION

This document provides methods and materials for treating a mammal (e.g., a human) having, or at risk of developing, muscular dystrophy (e.g., a LAMA2-RD). In some cases, this document provides AAV vectors designed for targeted gene activation of nucleic acid encoding a Lama1 polypeptide (e.g., the endogenous Lama1 gene). For example, an AAV vector designed for targeted gene activation of nucleic acid encoding a Lama1 polypeptide (e.g., the endogenous Lama1 gene) can have a genome (e.g., a single-stranded or double-stranded DNA genome) including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators. In some cases, AAV vectors provided herein (e.g., one or more AAV vectors designed for targeted gene activation of nucleic acid encoding a Lama1 polypeptide) can use CRISPRa to increase expression of a Lama1 polypeptide within a cell (e.g., within a cell within a mammal such as a human). In some cases, one or more AAV vectors provided herein (e.g., a single AAV vector designed for targeted gene activation of nucleic acid encoding a Lama1 polypeptide) can be administered to a mammal (e.g., a human) having, or at risk of developing, muscular dystrophy (e.g., a LAMA2-RD) to treat the mammal. For example, a single AAV vector provided herein (e.g., a single AAV vector designed for targeted gene activation of nucleic acid encoding a Lama1 polypeptide) can be used to increase expression of a Lama1 polypeptide by cells within a mammal (e.g., a human) having, or at risk of developing, muscular dystrophy (e.g., a LAMA2-RD) to treat the mammal.

Any appropriate AAV can be designed for targeted gene activation of nucleic acid encoding a Lama1 polypeptide (e.g., can be designed to have a genome including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide, and (b) a nucleic acid encoding a dCas polypeptide including one or more transcriptional activators). In some cases, an AAV vector designed for targeted gene activation of nucleic acid encoding a Lama1 polypeptide can have a single-stranded or double-stranded DNA genome. When containing a single-stranded DNA genome, the AAV vector designed for targeted gene activation of nucleic acid encoding a Lama1 polypeptide can be a positive-strand virus or a negative-strand virus. In some cases, an AAV vector designed for targeted gene activation of nucleic acid encoding a Lama1 polypeptide can infect dividing cells. In some cases, an AAV vector designed for targeted gene activation of nucleic acid encoding a Lama1 polypeptide can infect non-dividing cells. Examples AAV vectors that can be designed for targeted gene activation of nucleic acid encoding a Lama1 polypeptide as described herein (e.g., designed to have a genome including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide, and (b) a nucleic acid encoding a dCas polypeptide including one or more transcriptional activators) include, without limitation, serotype 9 AAV (AAV9) vectors, AAV6 vectors, AAV8 vectors, and AAV2 vectors.

An AAV vector provided herein (e.g., an AAV vector designed for targeted gene activation of nucleic acid encoding a Lama1 polypeptide) can include any appropriate nucleic acid encoding a gRNA that is complementary to a target site upstream of a transcriptional start site of an endogenous nucleic acid sequence encoding a Lama1 polypeptide. For example, an AAV vector having a genome (e.g., a single-stranded or double-stranded DNA genome) including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators can include any appropriate nucleic acid encoding a gRNA that is complementary to any appropriate target site upstream of a transcriptional start site of an endogenous nucleic acid sequence encoding a Lama1 polypeptide.

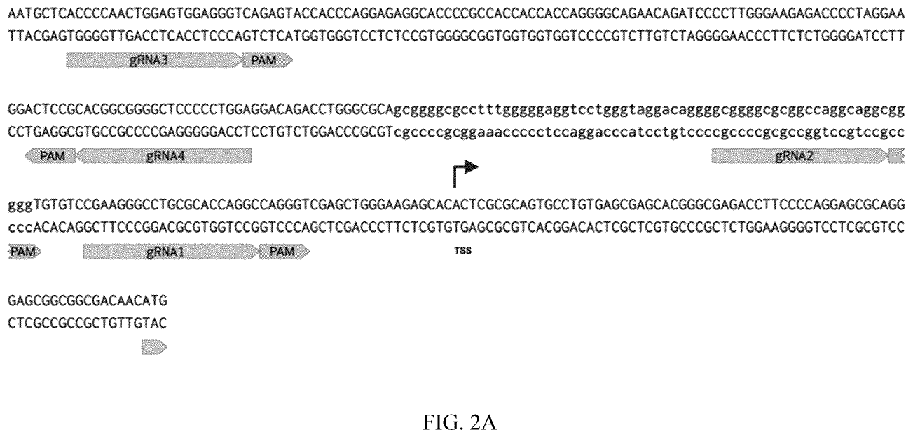

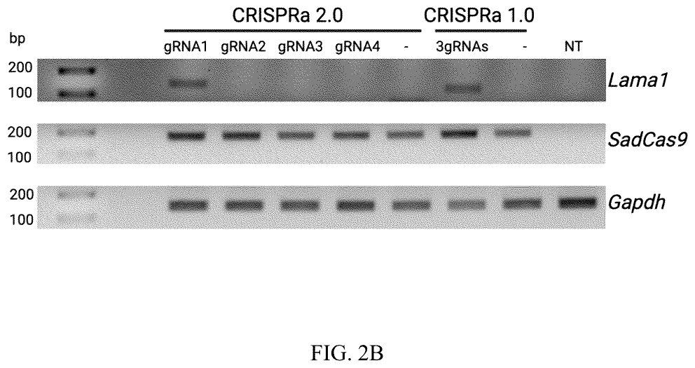

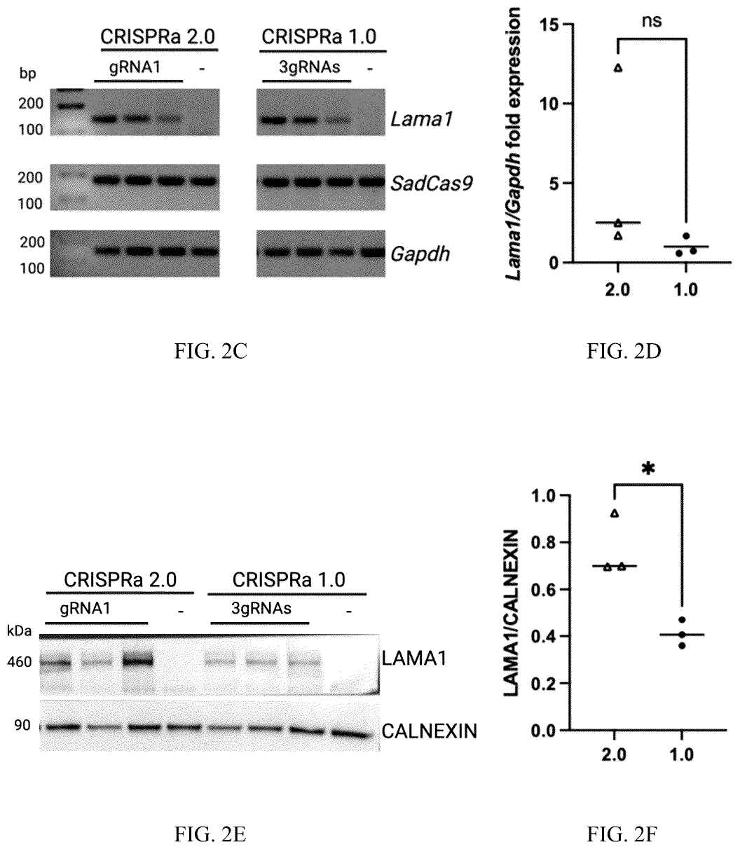

A nucleic acid encoding a gRNA that is complementary to a target site upstream of a transcriptional start site of an endogenous nucleic acid sequence encoding a Lama1 polypeptide in an AAV vector provided herein (e.g., an AAV vector designed for targeted gene activation of nucleic acid encoding a Lama1 polypeptide) can be complementary to any appropriate target site upstream of a transcriptional start site of an endogenous nucleic acid sequence encoding a Lama1 polypeptide. For example, an AAV vector having a genome (e.g., a single-stranded or double-stranded DNA genome) including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators can include a nucleic acid encoding a gRNA that is complementary to any appropriate target site upstream of a transcriptional start site of an endogenous nucleic acid sequence encoding a Lama1 polypeptide. In some cases, a target site upstream of a transcriptional start site of an endogenous nucleic acid sequence encoding a Lama1 polypeptide can be include at least a portion of an endogenous promoter sequence that can drive expression of an endogenous nucleic acid sequence encoding a Lama1 polypeptide (e.g., an endogenous Lama1 gene). In some cases, a target site upstream of a transcriptional start site of an endogenous nucleic acid sequence encoding a Lama1 polypeptide can be at least a portion of a genomic sequence located between 0 and 1000 nucleotides (e.g., between 0 and 750, between 0 and 500, between 0 and 300, between 0 and 250, between 0 and 200, between 0 and 150, between 0 and 100, between 0 and 50, between 50 and 1000, between 100 and 1000, between 150 and 1000, between 200 and 1000, between 250 and 1000, between 300 and 1000, between 400 and 1000, between 500 and 1000, between 0 and 1000, between 750 and 1000, between 50 and 750, between 100 and 500, between 150 and 300, between 50 and 100, between 100 and 250, between 250 and 500, between 300 and 600, between 400 and 700, or between 600 and 800 nucleotides) upstream of a transcriptional start site of an endogenous nucleic acid sequence encoding a Lama1 polypeptide within a mammal. For example, a target site upstream of a transcriptional start site of an endogenous nucleic acid sequence encoding a Lama1 polypeptide can be at least a portion of a genomic sequence located between 0 and 500 nucleotides upstream of a transcriptional start site of an endogenous nucleic acid sequence encoding a Lama1 polypeptide within a mammal. Examples of genomic sequences located between 0 and 1000 nucleotides upstream of a transcriptional start site of an endogenous nucleic acid sequence encoding a Lama1 polypeptide within a mammal include, without limitation, those sequences set forth in the National Center for Biotechnology Information (NCBI) databases at Accession Nos. AJ519495 and NG_034251.1. In some cases, a target site upstream of a transcriptional start site of an endogenous nucleic acid sequence encoding a Lama1 polypeptide can be at least a portion of the sequence shown in FIG. 2A.

A nucleic acid encoding a gRNA that is complementary to a target site upstream of a transcriptional start site of an endogenous nucleic acid sequence encoding a Lama1 polypeptide in an AAV vector provided herein (e.g., an AAV vector designed for targeted gene activation of nucleic acid encoding a Lama1 polypeptide) can encode a gRNA having any appropriate amount of complementarity to a target site upstream of a transcriptional start site of an endogenous nucleic acid sequence encoding a Lama1 polypeptide. For example, an AAV vector having a genome (e.g., a single-stranded or double-stranded DNA genome) including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators can include a nucleic acid encoding a gRNA having any appropriate amount of complementarity to a target site upstream of a transcriptional start site of an endogenous nucleic acid sequence encoding a Lama1 polypeptide. In some cases, a gRNA that is complementary to a target site upstream of a transcriptional start site of an endogenous nucleic acid sequence encoding a Lama1 polypeptide in an AAV vector provided herein (e.g., an AAV vector designed for targeted gene activation of nucleic acid encoding a Lama1 polypeptide) can be at least 95 percent (e.g., about 95 percent, about 96 percent, about 97 percent, about 98 percent, about 99 percent, or 100 percent) complementary to a target site upstream of a transcriptional start site of an endogenous nucleic acid sequence encoding a Lama1 polypeptide.

A nucleic acid encoding a gRNA that is complementary to a target site upstream of a transcriptional start site of an endogenous nucleic acid sequence encoding a Lama1 polypeptide in an AAV vector provided herein (e.g., an AAV vector designed for targeted gene activation of nucleic acid encoding a Lama1 polypeptide) can encode a gRNA having any appropriate sequence. For example, an AAV vector having a genome (e.g., a single-stranded DNA genome) including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators can include a nucleic acid encoding a gRNA having any appropriate sequence. In some cases, a gRNA that is complementary to a target site upstream of a transcriptional start site of an endogenous nucleic acid sequence encoding a Lama1 polypeptide in an AAV vector provided herein (e.g., an AAV vector designed for targeted gene activation of nucleic acid encoding a Lama1 polypeptide) can have an RNA sequence set forth in any one of SEQ ID NOs: 1 to 21 (see, Table 1). In some cases, a gRNA that is complementary to a target site upstream of a transcriptional start site of an endogenous nucleic acid sequence encoding a Lama1 polypeptide in an AAV vector provided herein (e.g., an AAV vector designed for targeted gene activation of nucleic acid encoding a Lama1 polypeptide) can be a reverse/complement of a sequence set forth in any one of SEQ ID NOs: 1 to 21 (see, e.g., Table 1). In some cases, a gRNA that is complementary to a target site upstream of a transcriptional start site of an endogenous nucleic acid sequence encoding a Lama1 polypeptide in an AAV vector provided herein (e.g., an AAV vector designed for targeted gene activation of nucleic acid encoding a Lama1 polypeptide) can be a variant of a sequence set forth in any one of SEQ ID NOs: 1 to 21 (see, e.g., Table 1). A variant of a gRNA that is complementary to a target site upstream of a transcriptional start site of an endogenous nucleic acid sequence encoding a Lama1 polypeptide can have the nucleotide sequence of any one of SEQ ID NOs: 1 to 21 with one or more (e.g., e.g., one, two, three, four, five, six, seven, eight, nine, ten, or more) nucleotide deletions, additions, substitutions, or combinations thereof, provided that the variant retains the ability to bind a target site upstream of a transcriptional start site of an endogenous nucleic acid sequence encoding a Lama1 polypeptide. In some cases, a gRNA that is complementary to a target site upstream of a transcriptional start site of an endogenous nucleic acid sequence encoding a Lama1 polypeptide in an AAV vector provided herein (e.g., an AAV vector designed for targeted gene activation of nucleic acid encoding a Lama1 polypeptide) can be as set forth in Table 1.

TABLE 1
Exemplary gRNA sequences.

| | | SEQ ID |
|---|---|---|
| gRNA | RNA sequence | NO |
| gRNA1 | GAAGGGCCUGCGCACCAGGC | 1 |
| gRNA2 | GCGGGGCGCGGCCAGGCAGGC | 2 |
| gRNA3 | GACCCCAACUGGAGUGGAGGGU | 3 |
| gRNA4 | GCACGGCGGGGCUCCCCCUGGA | 4 |
| g1 | GGCGGGGCGGGGCGCAGCCG | 5 |
| g2 | CAAGCUGGGCGCCCCCGGGGG | 6 |
| g3 | AGGCCAAGCUGGGCGCCCCCG | 7 |
| g4 | GUCAGCCCGGCCUCCCCGACU | 8 |
| g5 | AGCCGGGGAGGCGGCCGCGGU | 9 |
| g6 | GCAGCCCAGUUUCUCCUCCCC | 10 |
| g7 | CCGGACCCCCGCAGCCCAGUU | 11 |
| g8 | CCUCCCGGACCCCCGCAGCCC | 12 |
| g9 | UCAGCUGGGCACAUAGUUUCU | 13 |
| g10 | GCAGGGCCGCGGCGGGGGGGG | 14 |
| g11 | CCCAGCUCCUGGCAGGGGCGC | 15 |
| g12 | CCGAGCCUGGGUGGCUUCCCG | 16 |
| g13 | AGGGCGGCGGGUCGGCCGAGC | 17 |
| g14 | CUGCCCACGGCGGAAAGGGCG | 18 |
| g15 | AAGUCCUCUGCCCACGGCGGA | 19 |
| g16 | UUUCAGAAAAAAUCCUCAAGU | 20 |
| g17 | GCGUCCUCUCUCUCCAGCAGU | 21 |

In some cases, a gRNA that is complementary to a target site upstream of a transcriptional start site of an endogenous nucleic acid sequence encoding a Lama1 polypeptide in an AAV vector provided herein (e.g., an AAV vector designed for targeted gene activation of nucleic acid encoding a Lama1 polypeptide) can have the RNA sequence set forth in SEQ ID NO:1.

A nucleic acid encoding a gRNA that is complementary to a target site upstream of a transcriptional start site of an endogenous nucleic acid sequence encoding a Lama1 polypeptide in an AAV vector provided herein (e.g., an AAV vector designed for targeted gene activation of nucleic acid encoding a Lama1 polypeptide) can have any appropriate sequence. For example, an AAV vector having a genome (e.g., a single-stranded or double-stranded DNA genome) including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators can include a nucleic acid having any appropriate sequence. In some cases, a nucleic acid encoding a gRNA that is complementary to a target site upstream of a transcriptional start site of an endogenous nucleic acid sequence encoding a Lama1 polypeptide in an AAV vector provided herein (e.g., an AAV vector designed for targeted gene activation of nucleic acid encoding a Lama1 polypeptide) can have an RNA sequence set forth in any one of SEQ ID NOs: 22 to 42 set forth in Table 2.

TABLE 2
Nucleic acid sequences encoding
an exemplary gRNA.

| | | SEQ ID |
|---|---|---|
| gRNA | RNA sequence | NO |
| gRNA1 | GAAGGGCCTGCGCACCAGGC | 22 |
| gRNA2 | GCGGGGCGCGGCCAGGCAGGC | 23 |
| gRNA3 | GACCCCAACTGGAGTGGAGGGT | 24 |
| gRNA4 | GCACGGCGGGGCTCCCCCTGGA | 25 |
| g1 | GGCGGGGCGGGGCGCAGCCG | 26 |
| g2 | CAAGCTGGGCGCCCCCGGGGG | 27 |
| g3 | AGGCCAAGCTGGGCGCCCCCG | 28 |
| g4 | GTCAGCCCGGCCTCCCCGACT | 29 |
| g5 | AGCCGGGGAGGCGGCCGCGGT | 30 |
| g6 | GCAGCCCAGTTTCTCCTCCCC | 31 |
| g7 | CCGGACCCCCGCAGCCCAGTT | 32 |
| g8 | CCTCCCGGACCCCCGCAGCCC | 33 |
| g9 | TCAGCTGGGCACATAGTTTCT | 34 |
| g10 | GCAGGGCCGCGGCGGGGCGGG | 35 |
| g11 | CCCAGCTCCTGGCAGGGGCGC | 36 |
| g12 | CCGAGCCTGGGTGGCTTCCCG | 37 |
| g13 | AGGGCGGCGGGTCGGCCGAGC | 38 |
| g14 | CTGCCCACGGCGGAAAGGGCG | 39 |
| g15 | AAGTCCTCTGCCCACGGCGGA | 40 |
| g16 | TTTCAGAAAAAATCCTCAAGT | 41 |
| g17 | GCGTCCTCTCTCTCCAGCAGT | 42 |

In some cases, a nucleic acid encoding a gRNA that is complementary to any appropriate target site upstream of a transcriptional start site of an endogenous nucleic acid sequence encoding a Lama1 polypeptide in an AAV vector provided herein (e.g., an AAV vector designed for targeted gene activation of nucleic acid encoding a Lama1 polypeptide) can include one or more regulatory elements operably linked to the nucleic acid encoding the gRNA that is complementary to any appropriate target site upstream of a transcriptional start site of an endogenous nucleic acid sequence encoding a Lama1 polypeptide. For example, an AAV vector having a genome (e.g., a single-stranded or double-stranded DNA genome) including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators can include one or more regulatory elements operably linked to the nucleic acid encoding a gRNA that is complementary to any appropriate target site upstream of a transcriptional start site of an endogenous nucleic acid sequence encoding a Lama1 polypeptide. Such regulatory elements can include, without limitation, promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in an AAV vector provided herein (e.g., an AAV vector designed for targeted gene activation of nucleic acid encoding a Lama1 polypeptide) can depend on several factors, including, without limitation, inducibility, targeting, and the level of expression desired. For example, a promoter can be included in an AAV vector provided herein (e.g., an AAV vector designed for targeted gene activation of nucleic acid encoding a Lama1 polypeptide) to facilitate transcription of a nucleic acid encoding a gRNA that is complementary to a target site upstream of a transcriptional start site of an endogenous nucleic acid sequence encoding a Lama1 polypeptide. A promoter can be a naturally occurring promoter or a recombinant promoter. A promoter can be ubiquitous or inducible (e.g., in the presence of tetracycline), and can affect the expression of a nucleic acid encoding a gRNA that is complementary to a target site upstream of a transcriptional start site of an endogenous nucleic acid sequence encoding a Lama1 polypeptide in a general or tissue-specific manner. In some cases, a promoter that can be used to drive expression of a gRNA that is complementary to a target site upstream of a transcriptional start site of an endogenous nucleic acid sequence encoding a Lama1 polypeptide in cells can be a type 3 RNA polymerase III promoter. Examples of promoters that can be used to drive expression of a gRNA that is complementary to a target site upstream of a transcriptional start site of an endogenous nucleic acid sequence encoding a Lama1 polypeptide in cells include, without limitation, U6 promoters, 7SK promoters, H1 promoters, and reverse/complements thereof. As used herein, the term “operably linked” as used with respect to a nucleic acid encoding a gRNA or a nucleic acid encoding a polypeptide refers to positioning of a regulatory element relative to the nucleic acid encoding a gRNA or a polypeptide in such a way as to permit or facilitate expression of the gRNA or polypeptide. For example, an AAV vector provided herein (e.g., an AAV vector designed for targeted gene activation of nucleic acid encoding a Lama1 polypeptide) can contain a promoter and nucleic acid encoding a gRNA that is complementary to a target site upstream of a transcriptional start site of an endogenous nucleic acid sequence encoding a Lama1 polypeptide. In this case, the promoter can be operably linked to a nucleic acid encoding a gRNA that is complementary to a target site upstream of a transcriptional start site of an endogenous nucleic acid sequence encoding a Lama1 polypeptide such that it drives expression of the gRNA in cells.

An AAV vector provided herein (e.g., an AAV vector designed for targeted gene activation of nucleic acid encoding a Lama1 polypeptide) can include any appropriate nucleic acid encoding a dCas polypeptide including one or more transcriptional activators. For example, an AAV vector having a genome (e.g., a single-stranded or double-stranded DNA genome) including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators can include any appropriate nucleic acid encoding a dCas polypeptide including one or more transcriptional activators.

A nucleic acid encoding a dCas polypeptide including one or more transcriptional activators in an AAV vector provided herein (e.g., a single AAV vector designed for targeted gene activation of nucleic acid encoding a Lama1 polypeptide) can encode any appropriate dCas polypeptide. For example, an AAV vector having a genome (e.g., a single-stranded or double-stranded DNA genome) including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators can include nucleic acid encoding any appropriate dCas polypeptide. Examples of dCas polypeptides that can be encoded by a nucleic acid encoding a dCas polypeptide including one or more transcriptional activators in an AAV vector provided herein (e.g., a single AAV vector designed for targeted gene activation of nucleic acid encoding a Lama1 polypeptide) include, without limitation, catalytically inactive Cas9 (dCas9) polypeptides (e.g., Staphylococcus aureus dCas9 (SadCas9) polypeptides, Streptococcus pyogenes dCas9 polypeptides, Campylobacter jejuni dCas9 polypeptides, and Neisseria meningitidis dCas9 polypeptides) and dCas12a polypeptides. In some cases, a dCas polypeptide that can be encoded by a nucleic acid encoding a dCas polypeptide including one or more transcriptional activators in an AAV vector provided herein (e.g., an AAV vector designed for targeted gene activation of nucleic acid encoding a Lama1 polypeptide) can have the amino acid sequence set forth in the NCBI databases at Accession Nos. WP_038431314.1, J7RUA5, QSY61382.1, and UQW79990.1. In some cases, a dCas polypeptide that can be encoded by a nucleic acid encoding a dCas polypeptide including one or more transcriptional activators in an AAV vector provided herein (e.g., an AAV vector designed for targeted gene activation of nucleic acid encoding a Lama1 polypeptide) can have the amino acid sequence set forth in SEQ ID NO:43. In some cases, a dCas polypeptide that can be encoded by a nucleic acid encoding a dCas polypeptide including one or more transcriptional activators in an AAV vector provided herein (e.g., an AAV vector designed for targeted gene activation of nucleic acid encoding a Lama1 polypeptide) can comprise, consist essentially of, or consist of the amino acid sequence set forth in SEQ ID NO:43. In some cases, a dCas polypeptide that can be encoded by a nucleic acid encoding a dCas polypeptide including one or more transcriptional activators in an AAV vector provided herein (e.g., an AAV vector designed for targeted gene activation of nucleic acid encoding a Lama1 polypeptide) can comprise, consist essentially of, or consist of the amino acid sequence set forth in SEQ ID NO:43 with one, two, three, four, five, six, seven, eight, nine, or ten amino acid deletions, additions, substitutions, or combinations thereof. In some cases, a dCas polypeptide that can be encoded by a nucleic acid encoding a dCas polypeptide including one or more transcriptional activators in an AAV vector provided herein (e.g., a single AAV vector designed for targeted gene activation of nucleic acid encoding a Lama1 polypeptide) can comprise, consist essentially of, or consist of the amino acid sequence set forth in SEQ ID NO:43 with two or less, three or less, four or less, five or less, six or less, seven or less, eight or less, nine or less, or ten or less amino acid deletions, additions, substitutions, or combinations thereof.

A nucleic acid encoding a dCas polypeptide including one or more transcriptional activators in an AAV vector provided herein (e.g., an AAV vector designed for targeted gene activation of nucleic acid encoding a Lama1 polypeptide) can encode any appropriate one or more transcriptional activators. For example, an AAV vector having a genome (e.g., a single-stranded or double-stranded DNA genome) including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators can include nucleic acid encoding any appropriate one or more transcriptional activators. Examples of transcriptional activators that can be encoded by a nucleic acid encoding a dCas polypeptide including one or more transcriptional activators in an AAV vector provided herein (e.g., a single AAV vector designed for targeted gene activation of nucleic acid encoding a Lama1 polypeptide) include, without limitation, VP64 transcriptional activators (e.g., a VP64-Δp65-ΔRTA transcriptional activator), VP160 transcriptional activators, and VP32 transcriptional activators. In some cases, a transcriptional activator that can be encoded by a nucleic acid encoding a dCas polypeptide including one or more transcriptional activators in an AAV vector provided herein (e.g., a single AAV vector designed for targeted gene activation of nucleic acid encoding a Lama1 polypeptide) can have the amino acid sequence set forth in SEQ ID NO:44 (see, e.g., Example 2). In some cases, a transcriptional activator that can be encoded by a nucleic acid encoding a dCas polypeptide including one or more transcriptional activators in an AAV vector provided herein (e.g., an AAV vector designed for targeted gene activation of nucleic acid encoding a Lama1 polypeptide) can have the amino acid sequence set forth in the NCBI databases at Accession No. XP_016041658.1. In some cases, a transcriptional activator that can be encoded by a nucleic acid encoding a dCas polypeptide including one or more transcriptional activators in an AAV vector provided herein (e.g., an AAV vector designed for targeted gene activation of nucleic acid encoding a Lama1 polypeptide) can be as described elsewhere (see, e.g., Schmitz et al., The EMBO J., 10(12):3805-3817 (1991); Hardwick et al., J Virol., 66(9):5500-8 (1992); and Sadowski et al., Nature, 335(6190):563-4 (1988)).

In some cases, a nucleic acid encoding a dCas polypeptide including one or more transcriptional activators in an AAV vector provided herein (e.g., a single AAV vector designed for targeted gene activation of nucleic acid encoding a Lama1 polypeptide) can include one or more regulatory elements operably linked to the nucleic acid encoding the dCas polypeptide including one or more transcriptional activators. For example, an AAV vector having a genome (e.g., a single-stranded or double-stranded DNA genome) including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators can include one or more regulatory elements operably linked to the nucleic acid encoding a dCas polypeptide including one or more transcriptional activators. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in an AAV vector provided herein (e.g., a single AAV vector designed for targeted gene activation of nucleic acid encoding a Lama1 polypeptide) can depend on several factors, including, without limitation, inducibility, targeting, and the level of expression desired. For example, a promoter can be included in an AAV vector provided herein (e.g., a single AAV vector designed for targeted gene activation of nucleic acid encoding a Lama1 polypeptide) to facilitate transcription of a nucleic acid encoding a dCas polypeptide including one or more transcriptional activators. A promoter can be a naturally occurring promoter or a recombinant promoter. A promoter can be ubiquitous or inducible (e.g., in the presence of tetracycline), and can affect the expression of a nucleic acid encoding a dCas polypeptide including one or more transcriptional activators in a general or tissue-specific manner. In some cases, an AAV vector provided herein (e.g., a single AAV vector designed for targeted gene activation of nucleic acid encoding a Lama1 polypeptide) can contain a promoter and nucleic acid encoding a dCas polypeptide including one or more transcriptional activators. In this case, the promoter can be operably linked to a nucleic acid encoding a dCas polypeptide including one or more transcriptional activators such that it drives expression of the dCas polypeptide including one or more transcriptional activators in cells.

In some cases, a promoter that can be used to drive expression of a dCas polypeptide including one or more transcriptional activators in cells can include at least four (e.g., 4, 5, 6, 7, 8, 9, 10, or more) motifs having a DNA sequence of 5′-YGCGCANGCGCR-3′ (SEQ ID NO:45), each separated by an intervening nucleotide sequence. Examples of motifs having a DNA sequence of 5′-YGCGCANGCGCR-3′ (SEQ ID NO:45) are set forth in Table 3.

TABLE 3
Exemplary motifs having a DNA sequence
of 5′-YGCGCANGCGCR-3′ (SEQ ID NO: 45).

| DNA motif | SEQ ID NO |
|---|---|
| CGCGCATGCGCA | 46 |
| GTCGCATGCGCA | 47 |
| CGCGCAGGCGCG | 48 |
| TGCGCATGCGCG | 49 |
| TGCGCAAGCGCG | 50 |
| TGCGCATGCGCA | 51 |
| GGCGCCTGCGCA | 52 |
| CGCGCAGGCGCA | 53 |
| TGCGCAGGCGTG | 54 |
| GGCGCATGCGCA | 55 |

In some cases, a promoter that can be used to drive expression of a dCas polypeptide including one or more transcriptional activators in cells can include 10 motifs having a DNA sequence of 5′-YGCGCANGCGCR-3′ (SEQ ID NO:45), with each adjacent motif being separated by an intervening nucleotide sequence. For example, a promoter that can be used to drive expression of a dCas polypeptide including one or more transcriptional activators in cells can include motifs set forth in each of SEQ ID NOs: 46 to 55, with each adjacent motif being separated by an intervening nucleotide sequence. In some cases, a promoter that can be used to drive expression of a dCas polypeptide including one or more transcriptional activators in cells can include 4 motifs having a DNA sequence of 5′-YGCGCANGCGCR-3′ (SEQ ID NO:45), with each adjacent motif being separated by an intervening nucleotide sequence. For example, a promoter that can be used to drive expression of a dCas polypeptide including one or more transcriptional activators in cells can include motifs set forth in each of SEQ ID NOs: 46 to 49, with each adjacent motif being separated by an intervening nucleotide sequence.

A promoter that can be used to drive expression of a dCas polypeptide including one or more transcriptional activators in cells can include at least four (e.g., 4, 5, 6, 7, 8, 9, 10, or more) motifs having a DNA sequence of 5′-YGCGCANGCGCR-3′ (SEQ ID NO:45), with each adjacent motif being separated by an intervening nucleotide sequence, can include any appropriate intervening nucleotide sequences. An intervening nucleotide sequence can include any appropriate number of nucleotides. In some cases, an intervening nucleotide sequence can include from about 8 to about 12 nucleotides. Examples of intervening nucleotide sequences that can be present between each adjacent motif having a DNA sequence of 5′-YGCGCANGCGCR-3′ (SEQ ID NO:45) in a promoter that can be used to drive expression of a dCas polypeptide including one or more transcriptional activators in cells are set forth in Table 4.

TABLE 4
Exemplary intervening nucleotide sequence.

| DNA motif | SEQ ID NO |
|---|---|
| CCTCTGGGCC | 56 |
| CCAGTCTAGC | 57 |
| CTAGTCAGGC | 58 |
| CCATTGCCTG | 59 |
| CTACTCGCAC | 60 |
| CCTTTGCATG | 61 |
| CAATGGTCTG | 62 |
| CACGTCAGAC | 63 |
| CACGTCGGGC | 64 |

In some cases, a promoter that can be used to drive expression of a dCas polypeptide including one or more transcriptional activators in cells can include 10 motifs having a DNA sequence of 5′-YGCGCANGCGCR-3′ (SEQ ID NO:45), with each adjacent motif being separated by an intervening nucleotide sequence. For example, a promoter that can be used to drive expression of a dCas polypeptide including one or more transcriptional activators in cells can include 10 motifs having a DNA sequence of 5′-YGCGCANGCGCR-3′ (SEQ ID NO:45), with each adjacent motif being separated by an intervening nucleotide sequence set forth in each of SEQ ID NOs: 56 to 64. In some cases, a promoter that can be used to drive expression of a dCas polypeptide including one or more transcriptional activators in cells can include 4 motifs having a DNA sequence of 5′-YGCGCANGCGCR-3′ (SEQ ID NO:45), with each adjacent motif being separated by an intervening nucleotide sequence. For example, a promoter that can be used to drive expression of a dCas polypeptide including one or more transcriptional activators in cells can include 4 motifs having a DNA sequence of 5′-YGCGCANGCGCR-3′ (SEQ ID NO:45), with each adjacent motif being separated by an intervening nucleotide sequence set forth in each of SEQ ID NOs: 56 to 58.

In some cases, a promoter that can be used to drive expression of a dCas polypeptide including one or more transcriptional activators in cells can include the sequence set forth in SEQ ID NO:65 or a reverse/complement thereof.

In some cases, a promoter that can be used to drive expression of a dCas polypeptide including one or more transcriptional activators in cells can include the sequence set forth in SEQ ID NO:66 or a reverse/complement thereof.

In some cases, a promoter that can be used to drive expression of a dCas polypeptide including one or more transcriptional activators in cells can comprise, consist essentially of, or consist of one of the amino acid sequences set forth in Example 2. In some cases, a promoter that can be used to drive expression of a dCas polypeptide including one or more transcriptional activators in cells can comprise, consist essentially of, or consist of one of the amino acid sequences set forth in Example 2 with one, two, three, four, five, six, seven, eight, nine, or ten amino acid deletions, additions, substitutions, or combinations thereof. In some cases, a promoter that can be used to drive expression of a dCas polypeptide including one or more transcriptional activators in cells can comprise, consist essentially of, or consist of one of the amino acid sequences set forth in Example 2 with two or less, three or less, four or less, five or less, six or less, seven or less, eight or less, nine or less, or ten or less amino acid deletions, additions, substitutions, or combinations thereof.

In some cases, a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators within an AAV vector provided herein (e.g., a single AAV vector designed for targeted gene activation of nucleic acid encoding a Lama1 polypeptide) can be flanked by a first ITR sequence and a second ITR sequence. In some cases, a first ITR sequence can be directly or indirectly followed by a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators. In some cases, a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators can be directly or indirectly followed by a second ITR sequence. Examples of ITR sequences that can flank a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators within an AAV vector provided herein are set forth in Table 5.

TABLE 5
Exemplary ITR sequences.

| | SEQ ID |
|---|---|
| DNA motif | NO |
| CCTGCAGGCAGCTGCGCGCTCGCTCGCTC | 67 |
| ACTGAGGCCGCCCGGGCAAAGCCCGGGCG | |
| TCGGGCGACCTTTGGTCGCCCGGCCTCAG | |
| TGAGCGAGCGAGCGCGCAGAGAGGGAGTG | |
| GCCAACTCCATCACTAGGGGTTCCT | |
| GGACGTCCGTCGACGCGCGAGCGAGCGAG | 68 |
| TGACTCCGGCGGGCCCGTTTCGGGCCCGC | |
| AGCCCGCTGGAAACCAGCGGGCCGGAGTC | |
| ACTCGCTCGCTCGCGCGTCTCTCCCTCAC | |
| CGGTTGAGGTAGTGATCCCCAAGGA | |
| AGGAACCCCTAGTGATGGAGTTGGCCACT | 69 |
| CCCTCTCTGCGCGCTCGCTCGCTCACTGA | |
| GGCCGGGCGACCAAAGGTTGCCCGACGCC | |
| CGGGCTTTGCCCGGGCGGCCTCAGTGAGC | |
| GAGCGAGCGCGCAGCTGCCTGCAGG | |
| TCCTTGGGGATCACTACCTCAACCGGTGA | 70 |
| GGGAGAGACGCGCGAGCGAGCGAGTGACT | |
| CCGGCCCGCTGGTTTCCAACGGGCTGCGG | |
| GCCCGAAACGGGCCCGCCGGAGTCACTCG | |
| CTCGCTCGCGCGTCGACGGACGTCC | |

In some cases, a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators within an AAV vector provided herein (e.g., an AAV vector designed for targeted gene activation of nucleic acid encoding a Lama1 polypeptide) can be flanked by a first ITR sequence having a DNA sequence set forth in SEQ ID NO:67 and a second ITR sequence having DNA sequence set forth in SEQ ID NO:69.

In some cases, one or more AAV vectors provided herein (e.g., AAV vectors designed for targeted gene activation of nucleic acid encoding a Lama1 polypeptide) can be formulated into a composition (e.g., a pharmaceutically acceptable composition). For example, one or more AAV vectors provided herein (e.g., AAV vectors designed for targeted gene activation of nucleic acid encoding a Lama1 polypeptide) can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, a composition containing one or more AAV vectors provided herein (e.g., a population of a single AAV vector designed for targeted gene activation of nucleic acid encoding a Lama1 polypeptide) can be formulated for oral or parenteral (including, without limitation, an intramuscular, intrasciatic, intravenous, intradermal, intra-cerebral, intrathecal, or intraperitoneal (i.p.) injection) administration to the mammal. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

Also provided herein are nucleic acid molecules (e.g., isolated nucleic acid molecules) including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators.

Also provided herein are cells (e.g., host cells) containing one or more AAV vectors provided herein (e.g., host cells containing a population of a single AAV vector designed for targeted gene activation of nucleic acid encoding a Lama1 polypeptide). In some cases, a host cell containing one or more AAV vectors provided herein (e.g., a population of a single AAV vector designed for targeted gene activation of nucleic acid encoding a Lama1 polypeptide) can be an in vitro cell (e.g., a cell in an in vitro culture).

Also provided herein are cells (e.g., host cells) containing a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators. In some cases, a host cell containing a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators can be an in vitro cell (e.g., a cell in an in vitro culture).

Examples of cells (e.g., host cells) that can contain one or more AAV vectors provided herein (e.g., a population of a single AAV vector designed for targeted gene activation of nucleic acid encoding a Lama1 polypeptide) and/or a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide (e.g., an endogenous Lama1 gene), and (b) a nucleic acid encoding a dCas polypeptide including one or more transcriptional activators include, without limitation, muscle cells, fibroblasts, Schwann cells, and epithelial cells such as HEK293 cells.

Also provided herein are methods for using one or more AAV vectors provided herein (e.g., a single AAV vector designed for targeted gene activation of nucleic acid encoding a Lama1 polypeptide). In some cases, one or more AAV vectors provided herein (e.g., a single AAV vector designed for targeted gene activation of nucleic acid encoding a Lama1 polypeptide) can be administered to a mammal (e.g., a human) having, or at risk of developing, muscular dystrophy (e.g., a LAMA2-RD) to treat the mammal. For example, one or more AAV vectors provided herein (e.g., a single AAV vector designed for targeted gene activation of nucleic acid encoding a Lama1 polypeptide) can be used to increase expression of a Lama1 polypeptide by cells within a mammal (e.g., a human) having, or at risk of developing, muscular dystrophy (e.g., a LAMA2-RD) to treat the mammal.

In some cases, one or more AAV vectors provided herein (e.g., a single AAV vector designed for targeted gene activation of nucleic acid encoding a Lama1 polypeptide) can be administered to a mammal (e.g., a human) in need thereof (e.g., a human having, or at risk of developing, muscular dystrophy such as a LAMA2-RD) to increase expression of a Lama1 polypeptide by cells within the mammal. In some cases, the materials and methods described herein can be used to increase expression of a Lama1 polypeptide by cells within a mammal having, or at risk of developing, muscular dystrophy (e.g., a LAMA2-RD), by, for example, 10, 20, 30, 40, 50, 60, 70, 80, 90, 95, or more percent.

One or more AAV vectors provided herein (e.g., a single AAV vector designed for targeted gene activation of nucleic acid encoding a Lama1 polypeptide) can increase expression of a Lama1 polypeptide in any appropriate type of cells within a mammal (e.g., a human). In some cases, one or more AAV vectors designed for targeted gene activation of nucleic acid encoding a Lama1 polypeptide can increase expression of a Lama1 polypeptide in a muscle cell (e.g., a skeletal muscle cell). In some cases, one or more AAV vectors designed for targeted gene activation of nucleic acid encoding a Lama1 polypeptide can increase expression of a Lama1 polypeptide in a glial cell (e.g., a Schwann cell). In some cases, one or more AAV vectors designed for targeted gene activation of nucleic acid encoding a Lama1 polypeptide can increase expression of a Lama1 polypeptide in a fibroblast.

In some cases, one or more AAV vectors provided herein (e.g., a single AAV vector designed for targeted gene activation of nucleic acid encoding a Lama1 polypeptide) can be administered to a mammal (e.g., a human) in need thereof (e.g., a human having, or at risk of developing, muscular dystrophy such as a LAMA2-RD) to reduce or eliminate one or more symptoms of muscular dystrophy (e.g., a LAMA2-RD). Examples of symptoms of muscular dystrophy (e.g., a LAMA2-RD) include, without limitation, hypotonia, atrophy, contractures, spinal rigidity, white matter abnormalities, peripheral neuropathy, pulmonary dysfunctions, and paralysis. In some cases, the materials and methods described herein can be used to reduce one or more symptoms of muscular dystrophy (e.g., a LAMA2-RD) of within a mammal having, or at risk of developing, muscular dystrophy (e.g., a LAMA2-RD), by, for example, 10, 20, 30, 40, 50, 60, 70, 80, 90, 95, or more percent.

In some cases, one or more AAV vectors provided herein (e.g., a single AAV vector designed for targeted gene activation of nucleic acid encoding a Lama1 polypeptide) can be administered to a mammal (e.g., a human) in need thereof (e.g., a human having, or at risk of developing, muscular dystrophy such as a LAMA2-RD) to improve the survival of the mammal. In some cases, the materials and methods described herein can be used to improve survival of a mammal having, or at risk of developing, muscular dystrophy (e.g., a LAMA2-RD), by, for example, 10, 20, 30, 40, 50, 60, 70, 80, 90, 95, or more percent.

In some cases, one or more AAV vectors provided herein (e.g., a single AAV vector designed for targeted gene activation of nucleic acid encoding a Lama1 polypeptide) can be administered to a mammal (e.g., a human) in need thereof (e.g., a human having, or at risk of developing, muscular dystrophy such as a LAMA2-RD) to increase the body weight of the mammal. In some cases, the materials and methods described herein can be used to increase the body weight of a mammal having, or at risk of developing, muscular dystrophy (e.g., a LAMA2-RD), by, for example, 10, 20, 30, 40, 50, 60, 70, 80, 90, 95, or more percent.

Any appropriate mammal having, or at risk of developing, muscular dystrophy (e.g., a LAMA2-RD) can be treated as described herein (e.g., by administering one or more AAV vectors provided herein such as an AAV vector having genome including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide, and (b) a nucleic acid encoding a dCas polypeptide including one or more transcriptional activators). Examples of mammals that can have, or can be at risk of developing, muscular dystrophy (e.g., a LAMA2-RD) and that can be treated as described herein include, without limitation, humans, non-human primates (e.g., monkeys), dogs, cats, horses, cows, pigs, sheep, mice, rats, and rabbits. In some cases, a human having muscular dystrophy (e.g., a LAMA2-RD) can be treated as described herein. In some cases, the methods and materials described herein can be used in an animal other than a mammal (e.g., a zebrafish).

When treating a mammal (e.g., a human) having, or at risk of developing, muscular dystrophy (e.g., a LAMA2-RD) can be treated as described herein (e.g., by administering one or more AAV vectors provided herein such as an AAV vector having genome including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide, and (b) a nucleic acid encoding a dCas polypeptide including one or more transcriptional activators), the muscular dystrophy can be any type of muscular dystrophy. In some cases, a muscular dystrophy can be a LAMA2-RD.

In some cases, the methods described herein can include identifying a mammal (e.g., a human) as having, or as being at risk of developing, muscular dystrophy (e.g., a LAMA2-RD) by, for example, identifying that cells within the mammal include one or more mutations in an endogenous LAMA2 gene. Any appropriate method can be used to identify the presence of one or more mutations in an endogenous LAMA2 gene. For example, sequencing techniques (e.g., RNA seq) and/or PCR based techniques can be used to identify the presence of one or more mutations in an endogenous LAMA2 gene.

Any appropriate method can be used to deliver one or more AAV vectors provided herein (e.g., a population of a single AAV vector designed for targeted gene activation of nucleic acid encoding a Lama1 polypeptide) can be administered to a mammal (e.g., a human) having, or at risk of developing, muscular dystrophy (e.g., a LAMA2-RD). In some cases, a composition (e.g., a pharmaceutically acceptable composition) including one or more AAV vectors provided herein (e.g., a population of a single AAV vector designed for targeted gene activation of nucleic acid encoding a Lama1 polypeptide) can be administered locally or systemically. In some cases, a composition containing one or more AAV vectors provided herein (e.g., a population of a single AAV vector designed for targeted gene activation of nucleic acid encoding a Lama1 polypeptide) can be administered locally via intramuscular injection. In some cases, a composition containing one or more AAV vectors provided herein (e.g., a population of a single AAV vector designed for targeted gene activation of nucleic acid encoding a Lama1 polypeptide) can be administered locally via intrasciatic injection.

Any appropriate amount (e.g., any appropriate dose) of one or more AAV vectors provided herein (e.g., a population of a single AAV vector designed for targeted gene activation of nucleic acid encoding a Lama1 polypeptide) can be administered to a mammal (e.g., a human) having, or at risk of developing, muscular dystrophy (e.g., a LAMA2-RD). An effective amount of a composition containing one or more AAV vectors provided herein (e.g., a population of a single AAV vector designed for targeted gene activation of nucleic acid encoding a Lama1 polypeptide) can be any amount that can treat a mammal having muscular dystrophy (e.g., a LAMA2-RD) as described herein (e.g., that can increase expression of a Lama1 polypeptide by cells within the mammal) without producing significant toxicity to the mammal. In some cases, an effective amount of one or more AAV vectors provided herein (e.g., a population of a single AAV vector designed for targeted gene activation of nucleic acid encoding a Lama1 polypeptide) can be from about 5×10¹³ viral genomes per kilogram body weight (vg/kg) to about 2.0×10¹⁴ vg/kg. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the muscular dystrophy (e.g., a LAMA2-RD) in the mammal being treated may require an increase or decrease in the actual effective amount administered.

One or more AAV vectors provided herein (e.g., a population of a single AAV vector designed for targeted gene activation of nucleic acid encoding a Lama1 polypeptide) can be administered to a mammal (e.g., a human) having, or at risk of developing, muscular dystrophy (e.g., a LAMA2-RD) in any appropriate frequency. The frequency of administration can be any frequency that can treat a mammal having muscular dystrophy (e.g., a LAMA2-RD) as described herein (e.g., that can increase expression of a Lama1 polypeptide by cells within the mammal) without producing significant toxicity to the mammal. For example, the frequency of administration can be from about once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

One or more AAV vectors provided herein (e.g., a population of a single AAV vector designed for targeted gene activation of nucleic acid encoding a Lama1 polypeptide) can be administered to a mammal (e.g., a human) having, or at risk of developing, muscular dystrophy (e.g., a LAMA2-RD) for any appropriate duration. An effective duration for administering or using a composition containing one or more AAV vectors provided herein (e.g., AAV vectors designed for targeted gene activation of nucleic acid encoding a Lama1 polypeptide) can be any duration that can treat a mammal having muscular dystrophy (e.g., a LAMA2-RD) as described herein (e.g., that can increase expression of a Lama1 polypeptide by cells within the mammal) without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human) having, or at risk of developing, muscular dystrophy (e.g., a LAMA2-RD) can include administering to the mammal one or more AAV vectors provided herein (e.g., a population of a single AAV vector designed for targeted gene activation of nucleic acid encoding a Lama1 polypeptide) as the sole active ingredient to treat the mammal. For example, a composition containing one or more AAV vectors provided herein (e.g., a population of a single AAV vector designed for targeted gene activation of nucleic acid encoding a Lama1 polypeptide) can include the one or more AAV vectors provided herein (e.g., a population of a single AAV vector designed for targeted gene activation of nucleic acid encoding a Lama1 polypeptide) as the sole active ingredient in the composition that is effective to treat a mammal having, or at risk of developing, muscular dystrophy (e.g., a LAMA2-RD).

In some cases, methods for treating a mammal (e.g., a human) having, or at risk of developing, muscular dystrophy (e.g., a LAMA2-RD) as described herein (e.g., by administering one or more AAV vectors provided herein such as AAV vectors designed for targeted gene activation of nucleic acid encoding a Lama1 polypeptide) also can include administering to the mammal one or more (e.g., one, two, three, four, five or more) agents that can treat one or more symptoms of muscular dystrophy (e.g., a LAMA2-RD). Examples of agents that can be used to treat one or more symptoms of muscular dystrophy (e.g., a LAMA2-RD) and can be administered together with one or more AAV vectors provided herein (e.g., a population of a single AAV vector designed for targeted gene activation of nucleic acid encoding a Lama1 polypeptide) include, without limitation, corticosteroids (e.g., prednisone), angiotensin-converting enzyme (ACE) inhibitors, omigapil, losartan, anti-epileptic agents, and any combinations thereof. In cases where one or more AAV vectors provided herein (e.g., a population of a single AAV vector designed for targeted gene activation of nucleic acid encoding a Lama1 polypeptide) are used in combination with one or more agents used to treat one or more symptoms of muscular dystrophy (e.g., a LAMA2-RD), the one or more agents used to treat one or more symptoms of muscular dystrophy (e.g., a LAMA2-RD) can be administered at the same time (e.g., in a single composition containing both the one or more AAV vectors provided herein and the one or more agents used to treat one or more symptoms of muscular dystrophy such as a LAMA2-RD) or independently. For example, one or more AAV vectors provided herein (e.g., a population of a single AAV vector designed for targeted gene activation of nucleic acid encoding a Lama1 polypeptide) can be administered first, and the one or more agents used to treat one or more symptoms of muscular dystrophy (e.g., a LAMA2-RD) administered second, or vice versa.

In some cases, methods for treating a mammal (e.g., a human) having, or at risk of developing, muscular dystrophy (e.g., a LAMA2-RD) as described herein (e.g., by administering one or more AAV vectors provided herein such as AAV vectors designed for targeted gene activation of nucleic acid encoding a Lama1 polypeptide) also can include subjecting the mammal one or more (e.g., one, two, three, four, five or more) additional therapies used to treat muscular dystrophy (e.g., a LAMA2-RD). Examples of therapies that can be used to treat muscular dystrophy (e.g., a LAMA2-RD) include, without limitation, range-of-motion exercises, stretching exercises, low-impact aerobic exercise (e.g., walking and swimming), chest physiotherapy, cough machine, gastrostomy, and combinations thereof. In cases where one or more AAV vectors provided herein (e.g., a population of a single AAV vector designed for targeted gene activation of nucleic acid encoding a Lama1 polypeptide) are used in combination with one or more additional therapies used to treat muscular dystrophy (e.g., a LAMA2-RD), the one or more additional therapies can be performed at the same time or independently of the administration of one or more AAV vectors provided herein (e.g., a population of a single AAV vector designed for targeted gene activation of nucleic acid encoding a Lama1 polypeptide). For example, one or more AAV vectors provided herein (e.g., a population of a single AAV vector designed for targeted gene activation of nucleic acid encoding a Lama1 polypeptide) can be administered before, during, or after the one or more additional therapies are performed.

The invention will be further described in the following examples, which do not limit the scope of the invention described in the claims.

### EXAMPLES

### Example 1: A Miniaturized CRISPR Activation Enables a Single AAV Delivery to Upregulate LAMA1 for Congenital Muscular Dystrophy

This Example describes the design and generation of AAV vectors that drive expression of an endogenous nucleic acid encoding a Lama1 polypeptide (e.g., a Lama1 gene) in vivo resulting in expression of a Lama1 polypeptide within a mammal (e.g., within a cell within a mammal).

### Results

### The 4×NRF1 Promoter is Smaller and has the Desired Tissue Tropism

To reduce the size of the promoter driving the expression of CRISPRa, a synthetic 4×NRF1 promoter was evaluated. 4×NRF1 is a total of 78 nucleotides long including 4 spaced binding sites that recruit the Nuclear Respiratory Factor 1 (NRF1) transcriptional activator. The promoter's feasibility was first evaluated by testing its tissue tropism in different cells of interest. For this, plasmid constructs containing either a 4×NRF1 promoter sequence or a CMV (584 base pairs) promoter sequence driving expression of a nucleic acid sequence encoding mNeonGreen were used.

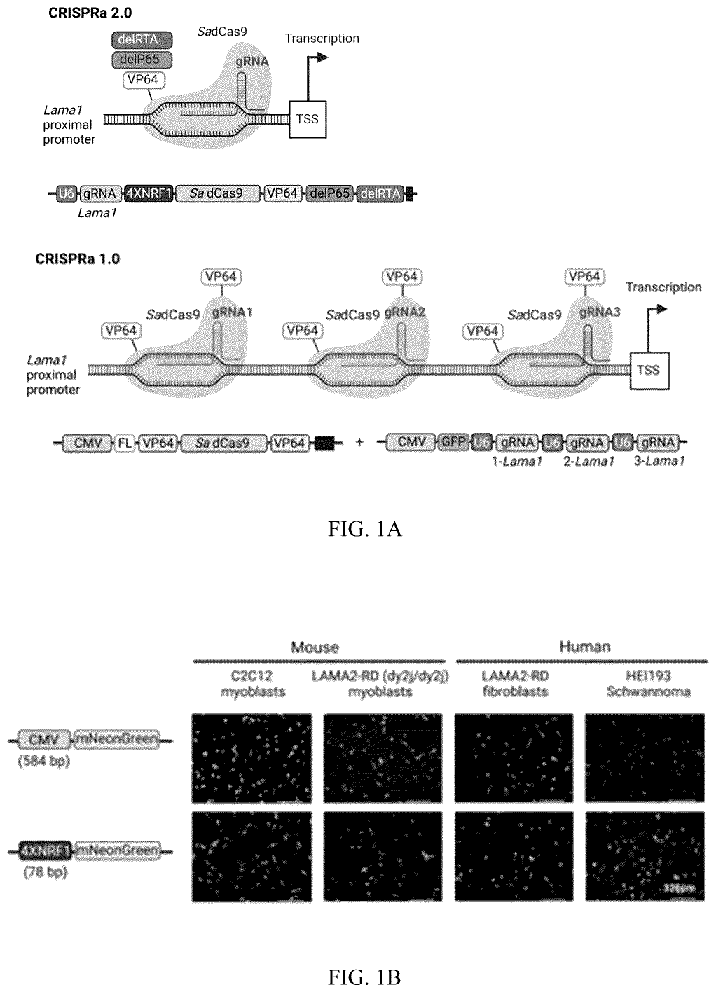

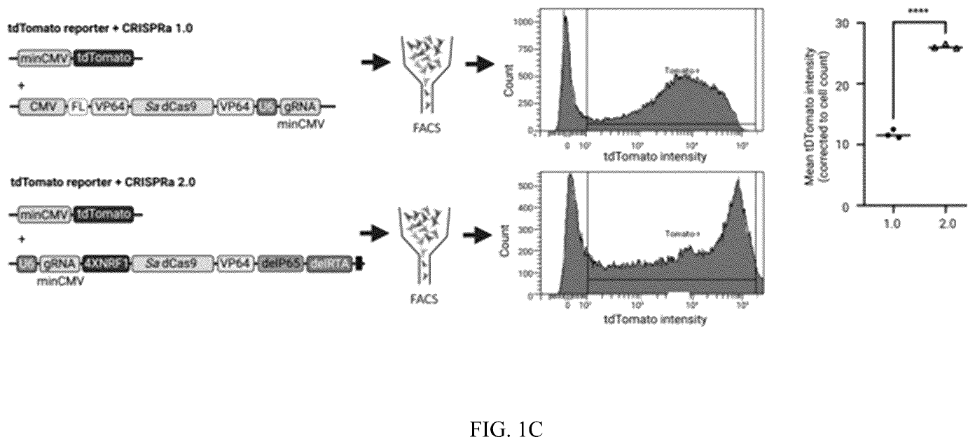

The constructs were transfected into 4 relevant cell lines (C2C12 murine myoblasts, LAMA2-RD dy^{2j}/dy^{2j} mouse myoblasts cells, HEI193 schwannoma cells, and LAMA2-RD patient-derived fibroblasts), and mNeonGreen expression was analyzed 24 hours post-transfection. It was observed that plasmids containing a 4×NRF1 promoter sequence drove mNeonGreen expression in the cell lines of interest (FIG. 1A), indicating its suitability as a small promoter to drive the expression of a CRISPRa system to treat LAMA2-RD.

### CRISPRa 2.0 has a Stronger Activation Strength in the Fluorescent Reporter Assay

After confirming the positive results of 4×NRF1 tissue tropism, the CRISPRa 2.0 was assembled. The starting backbone plasmid (pAAV-CMV-dSa-VPR mini.-2×snRP1 (Addgene #99688)) encodes for the CMV-driven SadCas9 with the miniaturized tripartite activators. The gRNA backbone (amplified from Addgene #135338) was then inserted via classical restriction enzyme XhoI digestion and sticky end ligation. Subsequently, the CMV promoter was swapped with 4×NRF1 via Gibson assembly. The final product generated (AAV-sgRNA-4×NRF1-SadCas9-miniVPR, also referred to herein as CRISPRa 2.0) has two BsaI restriction sites for the gRNA spacer sequence insertion.

The CRISPRa 2.0 system was tested in an in vitro miniCMV-driven tdTomato fluorescent reporter assay, where miniCMV cannot drive the expression of tdTomato without the help of an external transcriptional activator. HEK293T cells were co-transfected using two plasmids: the mini-CMV-tdTomato plasmid (gRNA_tdtomato_reporter_1_SA-2×) and a second plasmid with either CRISPRa 1.0 or 2.0, both with a gRNA targeting the upstream region of mini CMV. The presence and intensity of the tdTomato fluorescence were then analyzed via fluorescence-activated cell sorting (FACS) at 48 hours post-transfection. A significantly increased mean tdTomato intensity was observed in the CRISPRa 2.0 system (p<0.0001), suggesting that the new system is a stronger activator than the CRISPRa 1.0 (FIG. 1B).

### CRISPRa 2.0 can Successfully Upregulate Lama1 In Vitro

Different spacers targeting the upstream transcription start site of mouse Lama1 (between −50 bp and −400 bp) were inserted in the CRISPRa 2.0 construct (FIG. 2A). Final constructs were validated via Sanger sequencing and Oxford Nanopore reads. To select the optimal gRNA to upregulate Lama1, the upregulation efficiency of the original CRISPRa 1.0 system (consisting of 2 plasmids, one carrying the CRISPRa system and one with the 3 gRNAs) and the new CRISPRa 2.0 system (consisting of 1 plasmid) was tested in mouse C2C12 myoblasts. Considering that the CRISPRa 1.0 system uses a combination of three gRNAs, whereas CRISPRa 2.0 uses only 1 gRNA, each of the 3 gRNAs used in the CRISPRa 1.0 system was inserted in the CRISPRa 2.0 construct and tested separately. C2C12 myoblasts were electroporated and harvested at 72 hours post-transfection. RT-PCR results indicated that CRISPRa 2.0 with gRNA1 alone successfully upregulated Lama1 (FIG. 2B). Based on these results, subsequent studies focused on comparing the original CRISPRa 1.0 with 3 gRNAs and CRISPRa 2.0 with gRNA1 only.

Subsequently, the Lama1 upregulation potency of the new system was tested compared to the original system in vitro. For this, disease-relevant immortalized myoblasts from dy^{2j}/dy^{2j} mice were used. The cells were electroporated with either CRISPRa 1.0 or CRISPRa 2.0 and harvested at 72 hours post-transfection. Robust Lama1 upregulation in the dy^{2j}/dy^{2j} myoblasts was observed in both systems. Quantification of Lama1 transcript and LAMA1 polypeptide was done via qPCR and western blot densitometry analyses, respectively. Upon normalization to total mRNA/protein, no significant differences were observed between the systems at the mRNA level (FIGS. 2C and 2D). However, a significantly higher LAMA1 upregulation (p=0.0177) was observed in the new CRISPRa 2.0, with a 1.88-fold higher polypeptide levels as compared to CRISPRa 1.0 (FIGS. 2E and 2F). These in vitro results suggest that CRISPRa 2.0 is a suitable and improved CRISPRa system that can upregulate Lama1 expression using fewer gRNAs, indicating that a lower AAV9 dosage could be used for in vivo treatments.

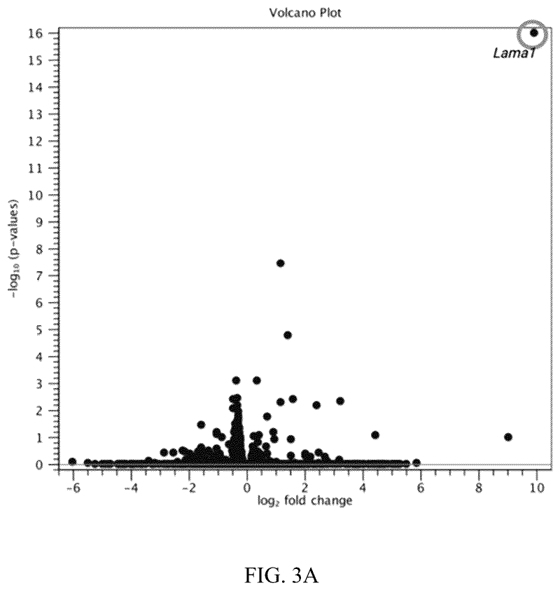

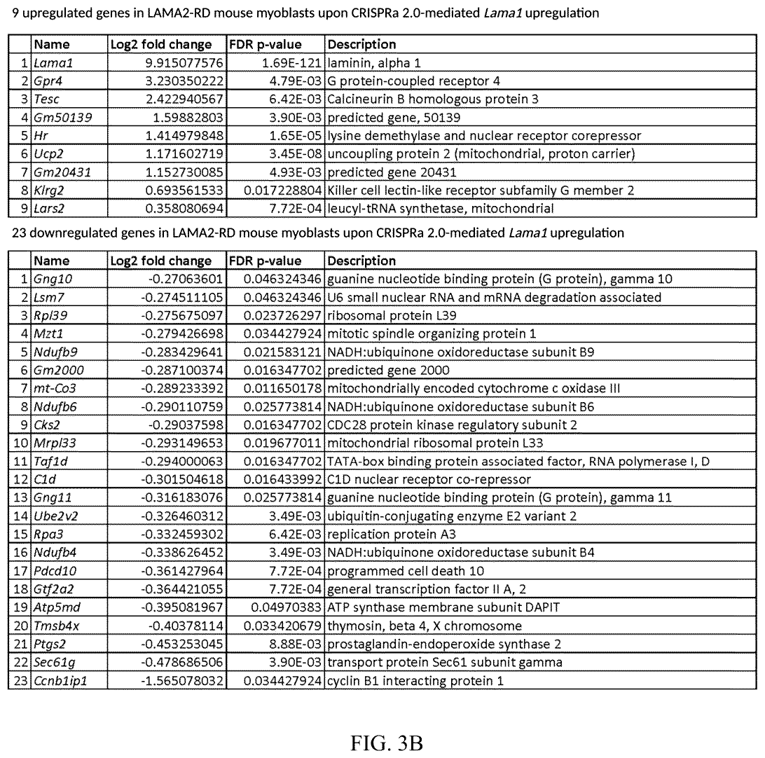

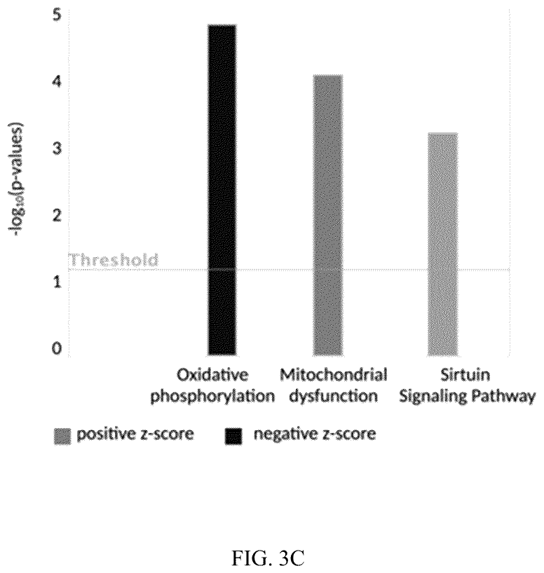

Bulk RNA-seq was carried out using RNA samples from the immortalized transfected myoblasts with the CRISPRa 2.0 system. Results further confirmed the robust upregulation of Lama1 (FIG. 3A). Thirty-two significantly differentially expressed genes, including Lama1, were observed in cells transfected with the CRISPRa 1.0 system (FIG. 3B). In cells transfected with the CRISPRa 1.0 system, 237 significantly differentially expressed genes were observed. In cells transfected with the CRISPRa 2.0 system, pathway enrichment analysis suggested that oxidative phosphorylation was downregulated, whereas mitochondrial dysfunction and sirtuin signaling pathway were increased after Lama1 upregulation (FIG. 3C).

AAV9-CRISPRa Delivery to Dy^{w}/Dy^{w} Mouse Muscles Leads to Lama1 Upregulation

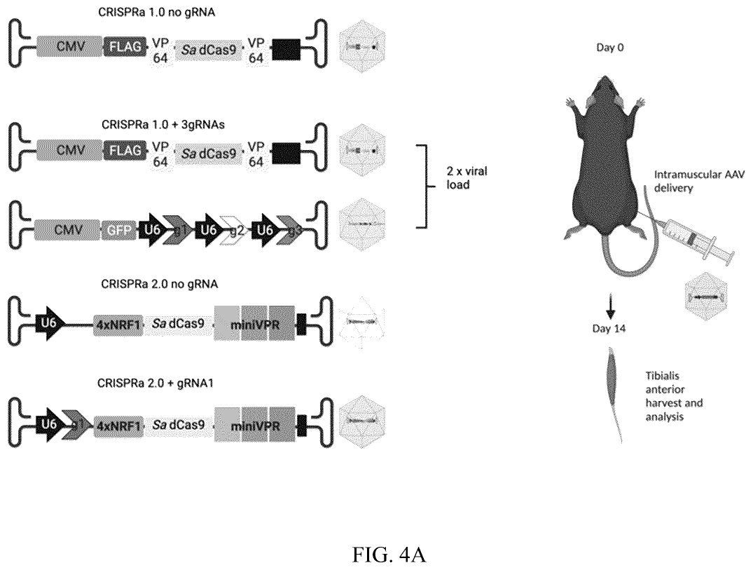

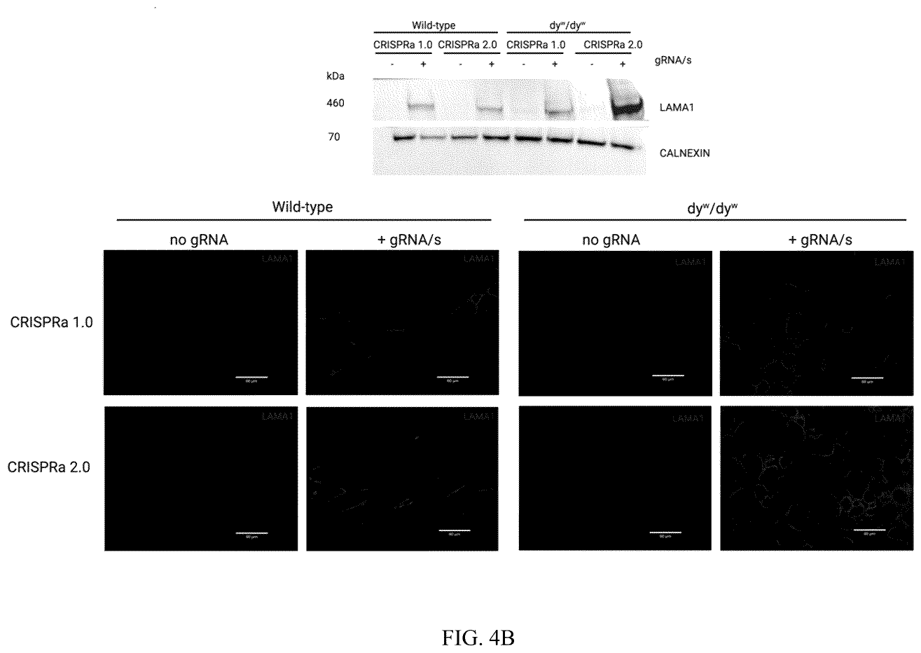

Following the positive results of the in vitro studies and to further confirm the efficacy of the new system, both CRISPRa systems with their corresponding gRNAs were packaged into AAV9 vectors. Then, dy^{w}/dy^{w} mice (Gawlik et al., Front. Mol. Neurosci., 13:59 (2020)) were injected intramuscularly with AAV9 carrying either CRISPRa 1.0 (with 3 gRNAs or no gRNA) or CRISPRa 2.0 (with gRNA1 or no gRNA) into their tibialis anterior muscles (FIG. 4A). Each mouse was injected with a single dose of 7.5E+11 GC AAV9 (1.5E+12 GC total AAV9 for CRISPRa 1.0 with 3 gRNAs due to the dual-AAV system) between 5 and 7 weeks of age. No abnormalities or adverse effects were observed in the mice during the 14 days following the injection. Mice were euthanized 14 days post-injection, and tibialis anterior muscles were harvested for analysis. The robust presence of LAMA1 polypeptide was only detected when the CRISPRa system plus corresponding gRNAs was delivered. Although no clear differences were observed in the wild-type mouse, western blot and immunofluorescence analyses of the dy^{w}/dy^{w} homozygous muscles isolated from the dyW mice revealed higher Lama1 upregulation using half the viral load (FIG. 4B).

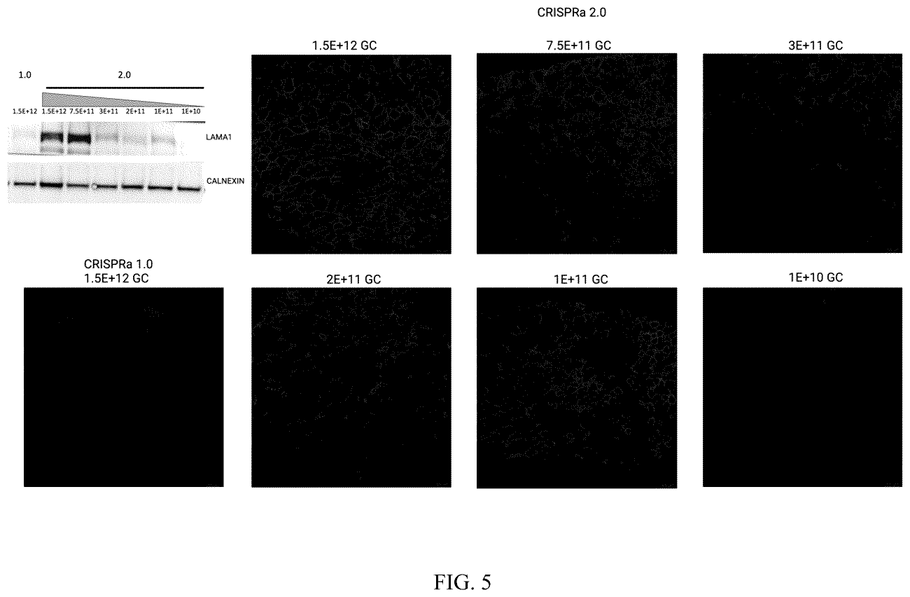

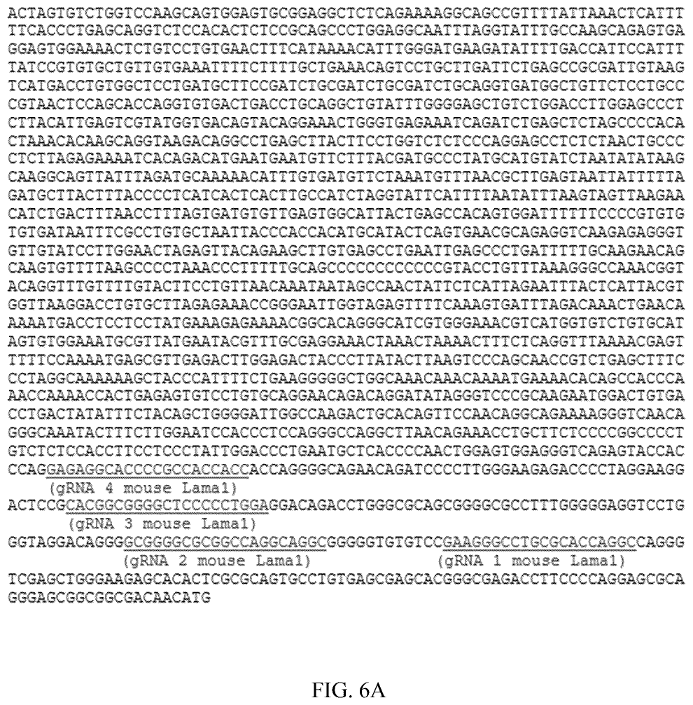

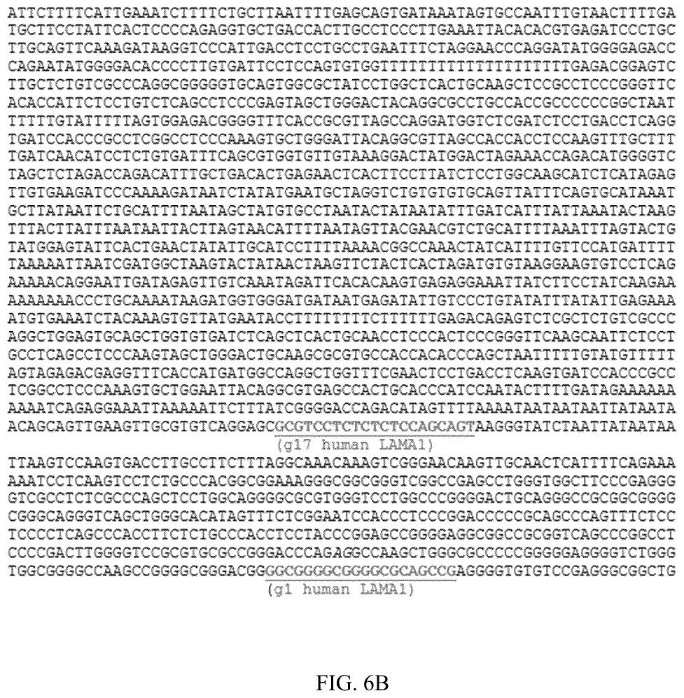

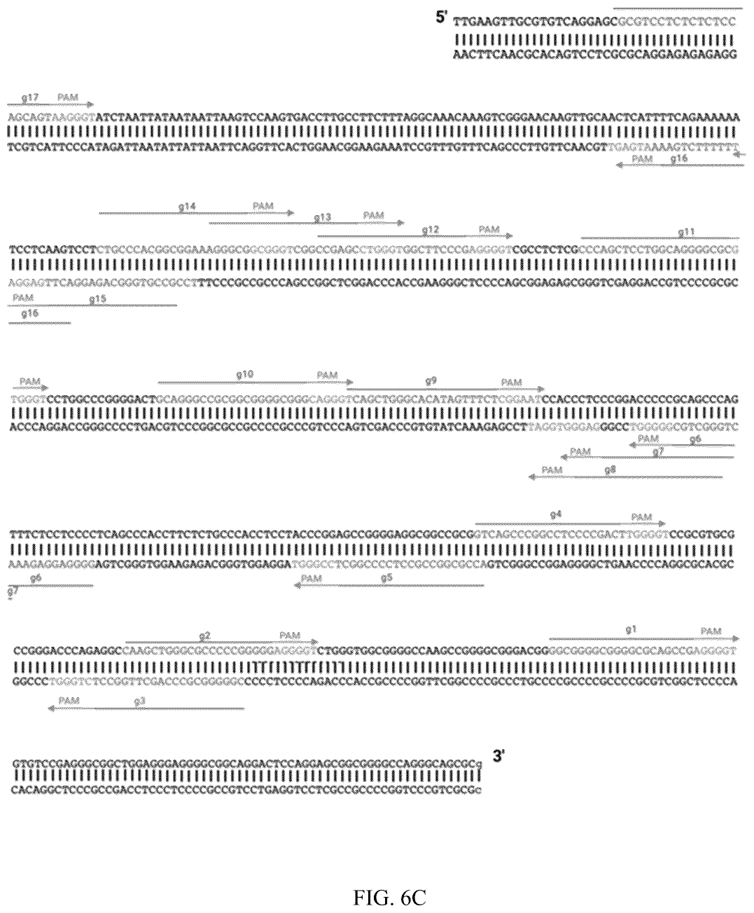

A titration test was performed to determine whether the viral dose of the CRISPRa 2.0 system could be reduced and still achieve a similar degree of upregulation as compared with the original CRISPRa 1.0 system. 6 different AAV9 loads of the CRISPRa 1.0 system were tested (1.5E+12 GC, 7.5E+11 GC, 3E+11 GC, 2E+11 GC, 1E+11 GC, and 1E+10 GC), and comparable LAMA1 upregulation was observed at a dose as low as 1E+11 GC (FIG. 5).

Together, these results demonstrate that one or more AAV vectors designed for targeted gene activation of nucleic acid encoding a Lama1 polypeptide (e.g., designed to have a genome including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide, and (b) a nucleic acid encoding a dCas polypeptide including one or more transcriptional activators) can be used to induce expression of a Lama1. For example, one or more AAV vectors designed for targeted gene activation of nucleic acid encoding a Lama1 polypeptide (e.g., designed to have a genome including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide, and (b) a nucleic acid encoding a dCas polypeptide including one or more transcriptional activators) can be used to increase a level of LAMA1 polypeptides in vivo resulting in expression of a Lama1 polypeptide within a mammal (e.g., within a cell within a mammal) having, or at risk of developing, muscular dystrophy (e.g., a LAMA2-RD) to treat the mammal.

### Material and Methods

### Cell Lines and Animals

HEK293T cells, C2C12 cells, HEI193 cells, and LAMA2-RD patient fibroblasts, immortalized dy²ⁱ/dy²ⁱ myoblasts (Kemaladewi et al., et al., Nature, 572:125-130 (2019)) were maintained in DMEM, high glucose glutamax supplemented with 10% FBS, and 1% penicillin-streptomycin (Gibco). Control fibroblasts were maintained in DMEM, 15% FBS, 1% L-glutamine, and 1% penicillin-streptomycin (Gibco). All cells were maintained at 37° C. and 5% CO₂.

dy^{w}/dy^{w} mice were purchased from the Jackson Laboratory. Both male and female mice were used in the study. The total number of experimental animals was based on TREAT-NMD standard operating protocol for MDC1A.

### 4×NRF1 Tissue Tropism Via Flow-Cytometry

A Neon Transfection System (ThermoFisher) was used to electroporate C2C12 cells (1650V, 10 ms, 3 pulses), HEI193 (950V, 30 ms, 2 pulses), dy^{2j}/dy^{2j} myoblasts (1400V, 20 ms, 2 pulses), and patient fibroblasts (1200V, 15 ms, 2 pulses) with a plasmid carrying a mNeonGreen gene under the control of either the 4×NRF1 promoter or a CMV promoter. Transfected cells were then cultured for 48 hours at 37° C. and 5% CO₂, after which the cells were imaged by fluorescent microscopy (ECHO Revolve).

### CRISPR-Activation 2.0 Assembly

CRISPR activation components were assembled via classical restriction enzyme ligation and Gibson Assembly methods. The starting backbone plasmid used was pAAV-CMV-dSa-VPR mini.-2×snRP1 (Addgene plasmid #99688). A gRNA backbone (amplified from Addgene #135338) was then inserted via classical restriction enzyme XhoI digestion and sticky end ligation. Subsequently, the CMV promoter was swapped with the 4×NRF1 promoter via Gibson Assembly. The final product generated, AAV-gRNA-4×NRF1-SadCas9-miniVPR (termed 4×NRF1-miniVPR) has two BsaI restriction sites used for gRNA spacer sequence insertion. Finally, the gRNA targeting mouse Lama1 was introduced into the spacer region of the AAV-gRNA-4×NRF1-SadCas9-miniVPR, to form the CRISPRa 2.0 construct. The guide RNAs targeting mouse Lama1 spacer sequences are listed in Table 6.

TABLE 6
Nucleic acid sequences that
encode gRNA sequences.

| Encoded | | SEQ ID |
|---|---|---|
| gRNA | Sequence 5′-3′ | NO |
| tdTomato | GTCCCCTCCACCCCACAGTG | 71 |
| gRNA | | |
| Lama1 | GAAGGGCCTGCGCACCAGGC | 22 |
| gRNA1 | | |
| Lama1 | GCGGGGCGCGGCCAGGCAGGC | 23 |
| gRNA2 | | |
| Lama1 | GACCCCAACTGGAGTGGAGGGT | 24 |
| gRNA3 | | |
| Lama1 | GCACGGCGGGGCTCCCCCTGGA | 25 |
| gRNA4 | | |

### CRISPRa 2.0 Upregulation Strength Validation Via Flow-Cytometry

Transfection of HEK293T cells with the reporter gRNA_tdtomato_reporter_1_SA-2× plasmid, alone (Addgene plasmid #79369) or in combination with either the original CRISPRa 1.0 or the CRISPRa 2.0 with a gRNA targeting the miniCMV, was performed using Lipofectamine® 3000 (ThermoFisher). Transfected cells were then cultured for 72 hours at 37° C. and 5% CO₂, after which the cells were trypsinized and sorted via FACS (BD FACSAria IIu) to quantify the average mean intensity of the tdTomato.

### RNA and Protein Expression Analysis of Lama1

C2C12 and dy²j/dy²j myoblasts were transfected as described herein and were cultured at 37° C. and 5% CO₂. 72 hours post-transfection RNA and protein were simultaneously harvested via Ambion PARIS kit (Invitrogen). cDNA synthesis was carried via the iScript Reverse Transcriptase Supermix kit (BioRad).

PCR amplification (Thermo fisher) was used to detect the presence of Lama1 upregulation for each of the CRISPRa systems. Primers targeting Lama1 exon 55 and junction of exons 55 and 56 were used for the amplification (Table 7).

qPCR was performed using Universal SYBR Green Fast qPCR (ABclonal) on a CFX96 Real-Time System (Bio-Rad). Lama1 expression was assessed using the same primers as the PCR amplification. CRISPRa expression was analyzed using primers targeting SadCas9 (Table 7). Endogenous Gapdh (Table 7) was used as an internal housekeeping gene control. ΔΔCₜ was used to assess fold change in expression between the different CRISPRa systems.

TABLE 7
Primer sequences.

| Sequence | | SEQ ID |
|---|---|---|
| name | Sequence 5′-3′ | NO |
| Lama1 | GGAAGGTTACAAAGTTCGAT | 72 |
| primer | TGG | |
| forward | | |
| Lama1 | ACGTGAAATAAGACCTTGCC | 73 |
| primer | ATC | |
| reverse | | |
| CRISPRa | AGCATGCCCGAGATCGAAAC | 74 |
| primer | | |
| forward | | |
| CRISPRa | TGTTGCCCTTGTCGTCCTTC | 75 |
| primer | | |
| reverse | | |
| Gapdh | TGTTTGTGATGGGTGTGAAC | 76 |
| primer | C | |
| forward | | |
| Gapdh | ACTGTGGTCATGAGCCCTTC | 77 |
| primer | | |
| reverse | | |

Total protein was quantified via Pierce BCA protein assay kit (Thermo fisher). For the western blot, protein samples were separated on NuPAGE 3 to 8% tris-acetate gels (Invitrogen) and transferred overnight at 150 mA onto 0.22 μm nitrocellulose membrane (Bio-Rad) via the Criterion Blotter (Bio-Rad) wet transfer using tris/glycine buffer with 20% methanol (Bio-Rad). The membranes were blocked in tris-buffered saline, 0.05% Tween (TBST), and 5% non-fat dry milk for 45 minutes at 4° C., followed by overnight incubation at 4° C. of the primary antibodies (rabbit anti-laminin α1 LNLEa (1 μg/mL) and rabbit polyclonal anti-Calnexin (abeam ab22595, 0.125 μg/mL)). Secondary antibodies (HRP anti-rabbit IgG (Bio-Rad 1706515) and HRP anti-mouse IgG (Bio-Rad 1706516)) were used to incubate the membrane for 1 hour at 4° C.

### RNA-Sequencing and Pathway Enrichment Analysis

Total RNA was isolated from dy^{2j}/dy^{2j} myoblasts using the Nucleospin RNA Plus kit (Macherey-Nagel). cDNA libraries were prepared via a 3′-Tag-RNA-Seq library kit (Illumina). Libraries were then sequenced with Hi-Seq 4000 platform with 2×58 bp pair-end reads.

Downstream quality control and analysis were carried out using the Qiagen-licensed CLC Genomic Workbench software version 22.0.1, the same software was used to obtain the differentially expressed genes (DEGs) and generate the volcano plot. Qiagen-licensed Ingenuity Pathway Analysis (IPA) software was then used for the pathway enrichment analysis of the DEGs.

### Viral Vector Production, AAV9 Injection, and Tissue Harvest

CRISPRa 1.0 and 2.0 plasmids were packaged into AAV9 vectors.

Tibialis anterior intramuscular injections were carried out in wild-type and dy^{w}/dy^{w} mice (ages ranging from 5 to 7 weeks) with either CRISPRa 1.0 (with 3 sgRNAs or no sgRNA) or CRISPRa 2.0 (with gRNA1 or no gRNA). For the initial experiment, each mouse was injected with a single dose of 7.5E+11 viral genome AAV9. For the CRISPRa 1.0 strategy, the mice received 2×7.5E+11 viral genome (totaling 1.5E+12 viral genome) due to the requirement for a dual AAV system. The tibialis anterior muscles were harvested two weeks post-injection, snap-frozen in isopentane submerged in liquid nitrogen, stored at −80° C. for downstream analysis. Subsequent titration studies followed the same process but used decreased levels of viral genome (1.5E+12 GC, 7.5E+11 GC, 3E+11 GC, 2E+11 GC, 1E+11 GC, and 1E+10 GC).

### Immunofluorescence Staining

Muscles were cryo-sectioned at 8 μm thickness and processed according to standard methods. Primary antibodies used were rabbit anti-laminin laminin α1 αE3 (1:500) and rat anti-laminin α2 (abeam ab11576, 1:500). Secondary antibodies used were goat anti-rabbit Alexa Fluor 594 (Thermo fisher A11012, 1:250) and goat anti-mouse 488 (Thermo fisher A11006, 1:250). Images were captured using Revolve microscope (ECHO) for epifluorescence images and Stellaris 5 (Leica) for confocal images.

### Statistical Analysis

Student's t test was used to assess differences between the CRISPRa 1.0 and 2.0 with GraphPad Prism 9.

### Example 2: CRISPRa 2.0-Mediated Lama1 Upregulation Ameliorates Major Disease Phenotypes In Vivo

This Example describes the use of systemic delivery of Lama1 via AAV9-CRISPRa 2.0 to treat LAMA2-related deficient congenital muscular dystrophy.

### Results

### AAV9-CRISPRa 2.0 Systemic Delivery

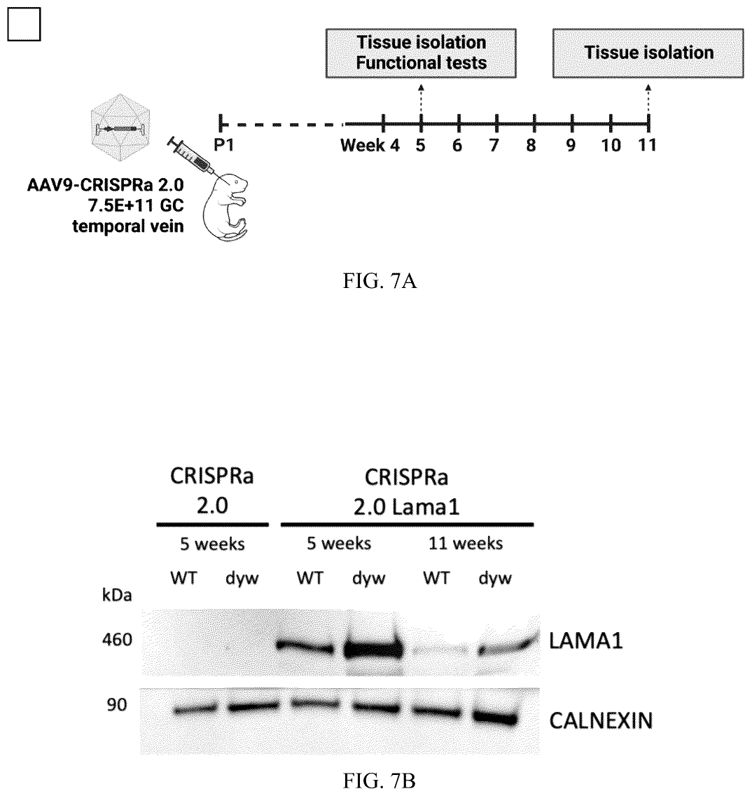

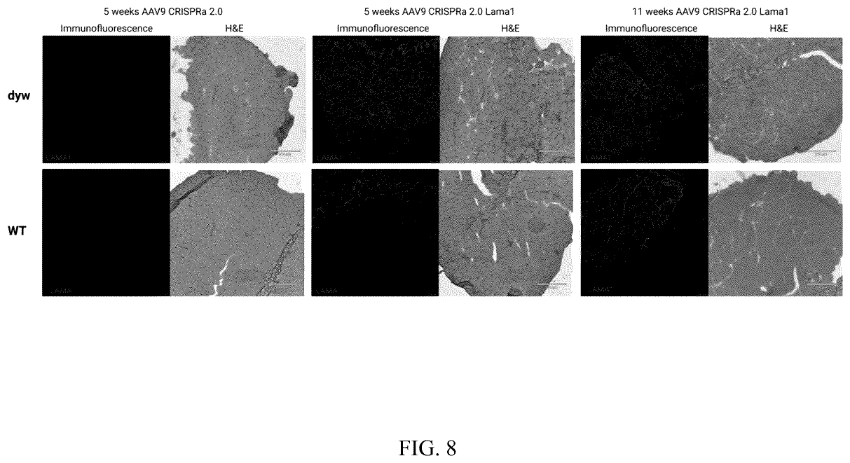

Pre-symptomatic, neonatal P1 dy^{w}/dy^{w} mice received CRISPRa 2.0 via temporal vein administration to determine whether CRISPRa 2.0 achieved systemic Lama1 upregulation and disease prevention (FIG. 7A). LAMA1 was detected at both 5 weeks and 11 weeks post-injection in the skeletal muscles (FIGS. 7B and 8), indicating that AAV9 successfully delivered the CRISPRa 2.0 system into the skeletal muscles. Hematoxylin and eosin staining of the muscles indicated improved muscle histopathology in the presence of LAMA1 (FIG. 8).

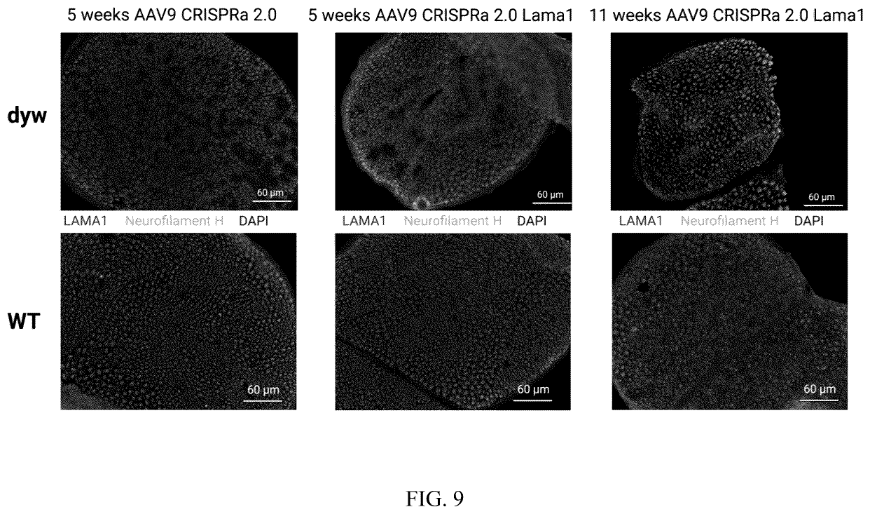

Because LAMA-RD affects not only the skeletal muscles but also the peripheral nerves, Lama1 expression in sciatic nerves was assessed (FIG. 9). In the nerves injected with AAV9 CRISPRa 2.0 Lama1, the expression of LAMA1 in the endoneurium area surrounding the axonal fibers was observed (FIG. 9). Similar to the skeletal muscles, LAMA1 expression in the nerves was detected at both 5 weeks and 11 weeks post-injection.

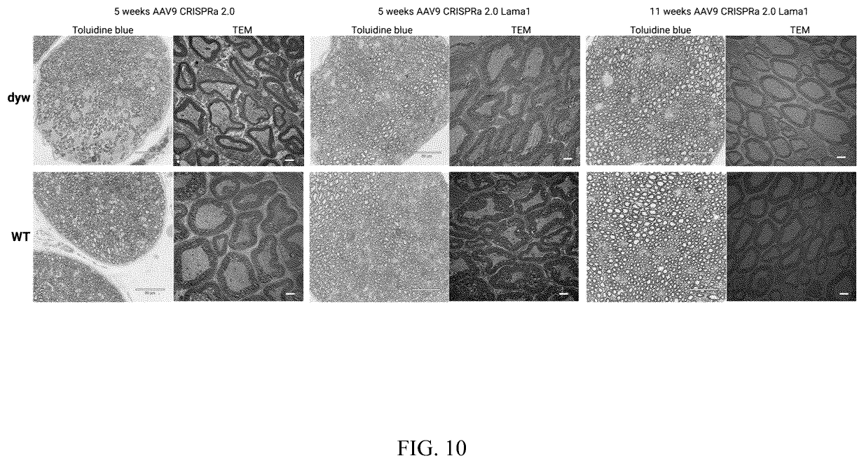

The morphology of the nerves was assessed using toluidine blue and transmission electron microscopy (TEM) to determine the impact of LAMA1 upregulation on the nerve structures (FIG. 10). dy^{w}/dy^{w} nerves with LAMA1 expression exhibited decreased areas of demyelination and thicker myelin layers in the toluidine blue-stained and the TEM-generated images, respectively.

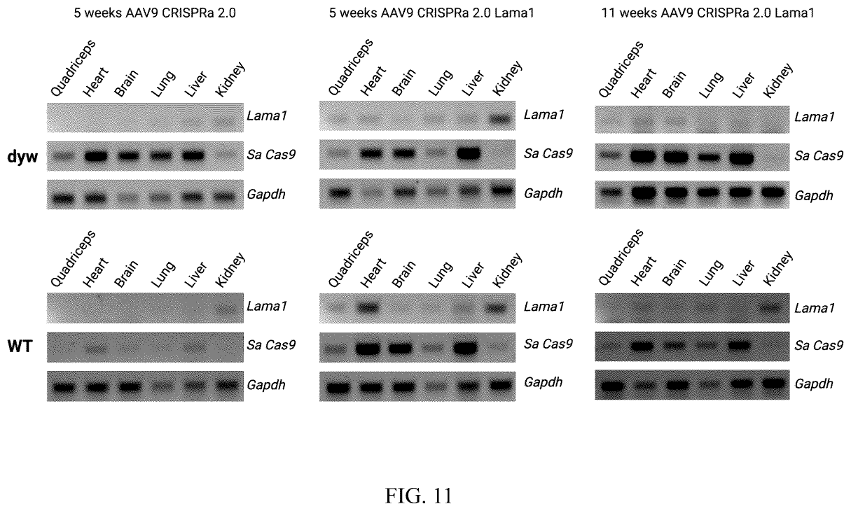

To determine the ability of CRISPRa 2.0, particularly when combined with 4×NRF1 promoter and delivered via AAV9, to activate Lama1 transcriptional machinery in tissues other than skeletal muscle and nerves, the expression of Lama1 and Sa dCas9 in the heart, brain, lungs, liver, and kidneys was assessed (FIG. 11). The expression of both transcripts was detected in all of the tissues analyzed with varying intensities. These results establish the previously unknown tissue targeting capability of the 4×NRF1 promoter in vivo and were necessary to rule out potential detrimental effects (or lack thereof) of Lama1 upregulation body-wide.

Overall, LAMA1 was only detected when CRISPRa 2.0 with Lama1 gRNA1 was administered. The presence of LAMA1 led to improvement in both muscle fiber and nerve histology and myelination. No apparent abnormalities in the gross morphology were observed in the injected wild-type mice.

### AAV9-CRISPRa 2.0 Systemic Delivery Leads to Improved Survival and Body Weight

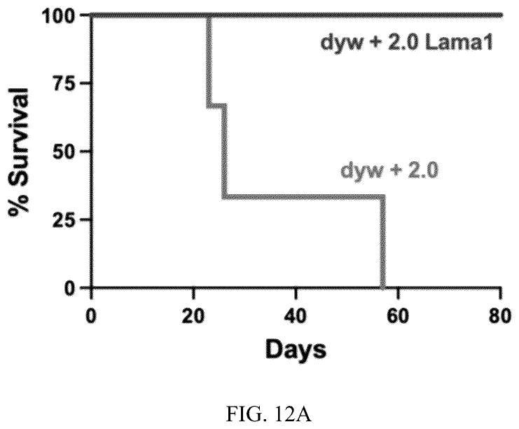

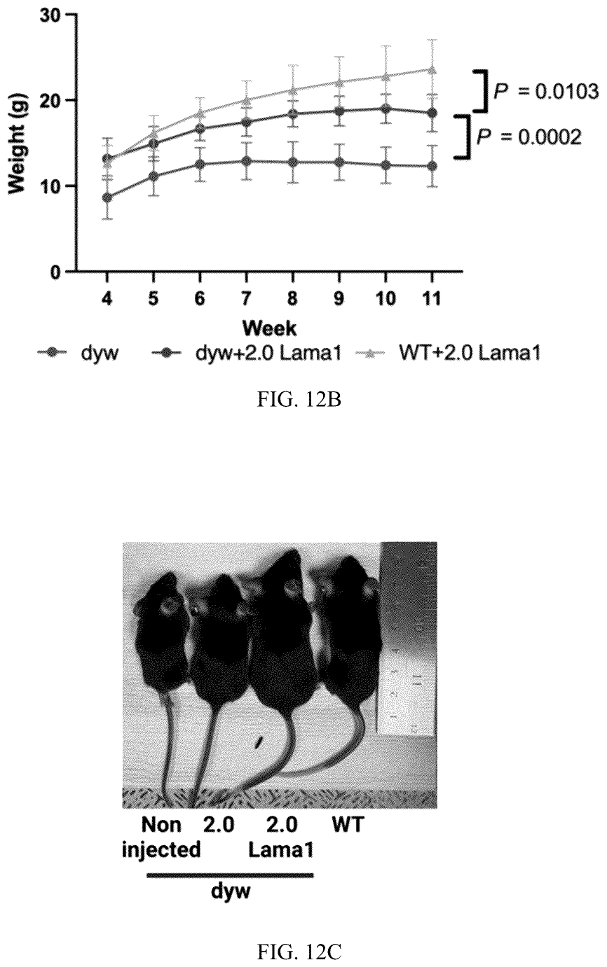

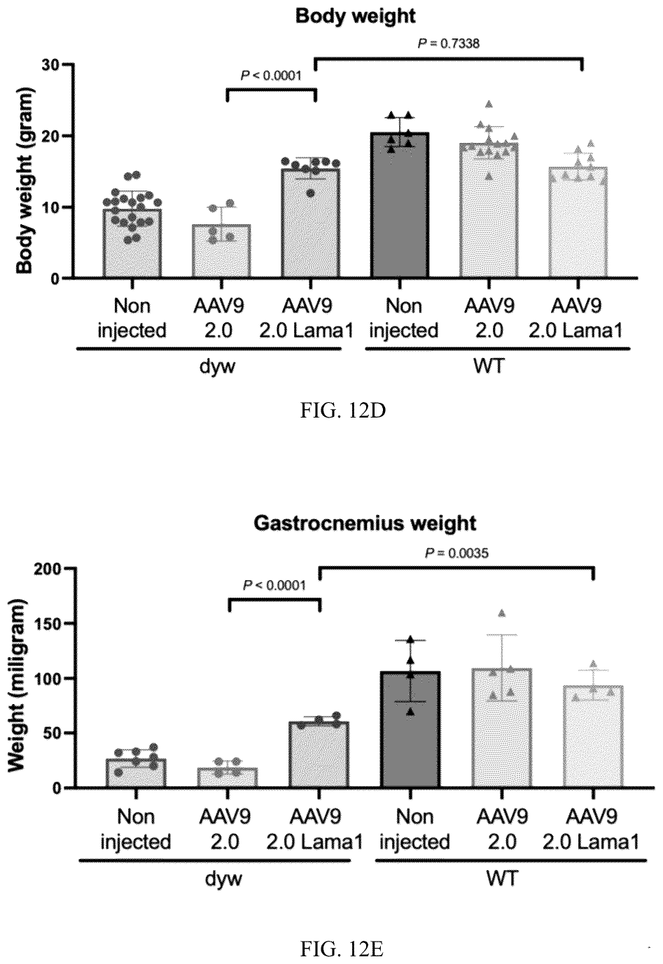

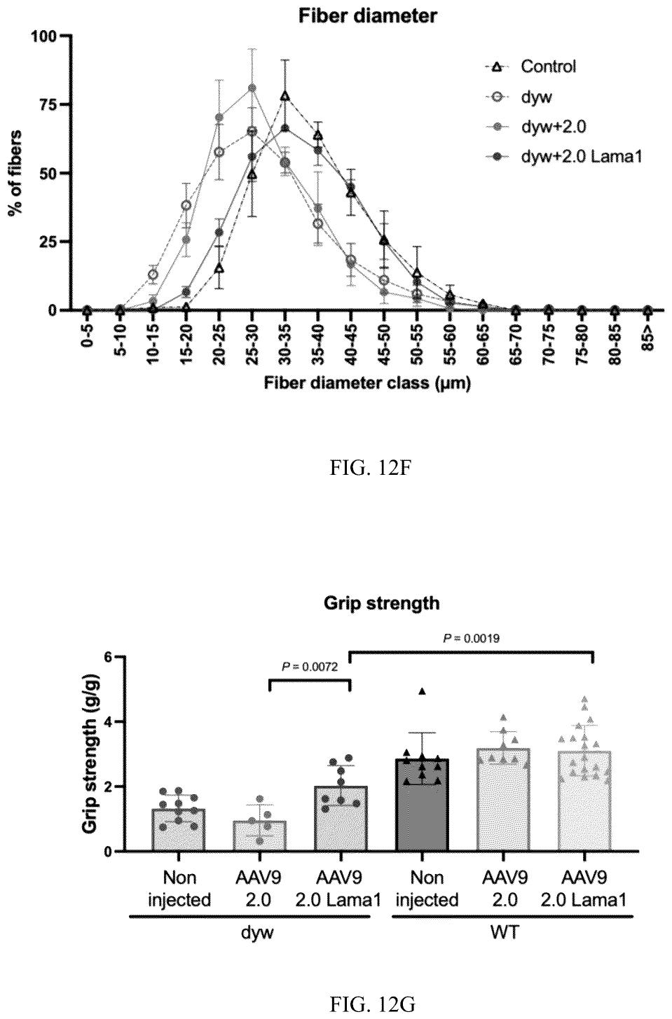

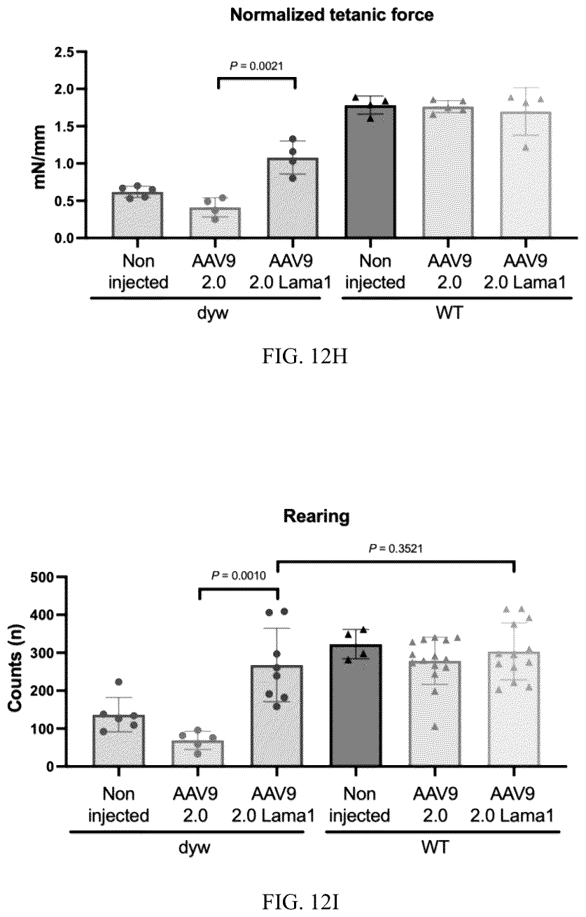

In addition to histopathology and molecular analyses, the physiological impact of LAMA1 upregulation was evaluated. Considering the short lifespan of the dy^{w}/dy^{w} mice, whether LAMA1 upregulation improves survival was assessed. The median survival of dy^{w}/dy^{w} mice was 8 weeks, which was similar to the survival of dy^{w}/dy^{w} mice injected with CRISPRa 2.0 no gRNA. The dy^{w}/dy^{w} mice injected with CRISPRa 2.0 with Lama1 gRNA1 and subsequently showed Lama1 upregulation survived up to the end of the study design, which was at 11 weeks (FIG. 12A).

In addition to improved survival, dy^{w}/dy^{w} mice treated with CRISPRa 2.0 with Lama1 gRNA1 and that were aged up to 11 weeks showed dramatic increases in body weight (FIGS. 12B-12D). There were no significant differences between the body weight of these treated dy^{w}/dy^{w} mice when compared to control wild-type mice until the 11-week mark (Table 8). Control dy^{w}/dy^{w} mice peaked at around 15 grams throughout the study period.

TABLE 8
Statistical analysis P values of single time point weights.

| | | dy^{w}/dy^{w} non-injected | dy^{w}/dy^{w} 2.0 Lama1 |
|---|---|---|---|
| | | vs. dy^{w}/dy^{w} 2.0 | vs. wild-type 2.0 |
| | | Lama1 | Lama1 |
| Week | 4 | 0.0119 | >0.9999 |
| | 5 | 0.0542 | 0.9851 |
| | 6 | 0.0254 | 0.8723 |
| | 7 | 0.0105 | 0.5511 |
| | 8 | 0.0008 | 0.4386 |
| | 9 | 0.0003 | 0.212 |
| | 10 | <0.0001 | 0.1137 |
| | 11 | 0.0002 | 0.0103 |

### AAV9-CRISPRa 2.0 Systemic Delivery Leads to Improvements in Hindlimb Paralysis

The effect of LAMA1 upregulation on the development of hindlimb paralysis was assessed. Hindlimb paralysis in untreated dy^{w}/dy^{w} mice began to appear at the age of 3 weeks and remained prevalent in any mice that survived past 7 weeks. The treated dy^{w}/dy^{w} mice showed either a delayed onset of hindlimb paralysis at 9 weeks (two out of four mice) or exhibited extremely mild signs of hindlimb paralysis at the end of the 11-week study (two out of four mice).

Treated dy^{w}/dy^{w} mice also showed significant improvement in body weight and gastrocnemius muscle weight compared to the untreated and control counterparts (FIGS. 12D-12E). Clear differences in the fiber size distribution, in which the treated dy^{w}/dy^{w} mice showed a shift of larger muscle fiber size towards the wild-type fibers, were detected (FIG. 12F). Moreover, the dy^{w}/dy^{w} mice treated with AAV9 CRISPRa 2.0 Lama1 show significant improvement in muscle strength (FIG. 12G), force (FIG. 12H), and rearing activity (FIG. 12I), compared to the AAV9 CRISPRa 2.0-injected dy^{w}/dy^{w} mice. The extent of some of these improvements, namely body weight and rearing activities, reached the same physiological levels as wild-type counterparts, indicating successful rescue of the disease phenotypes upon treatment.

### Delayed Treatment Improves Survival and Mobility

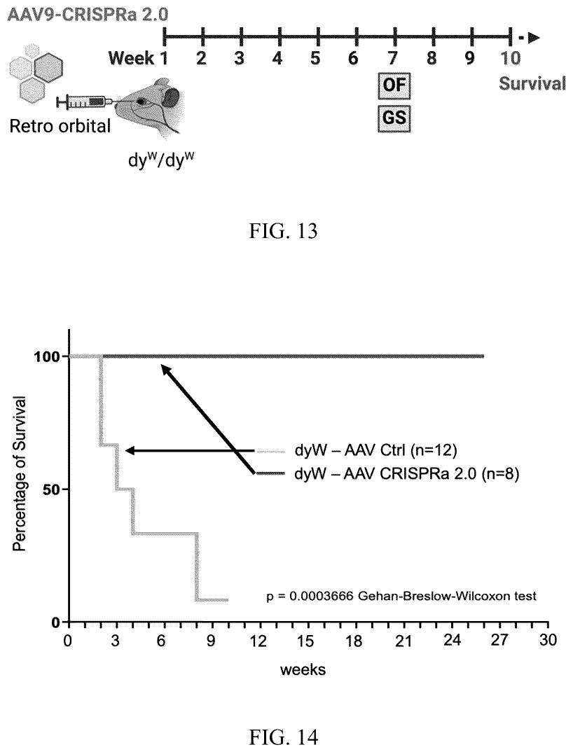

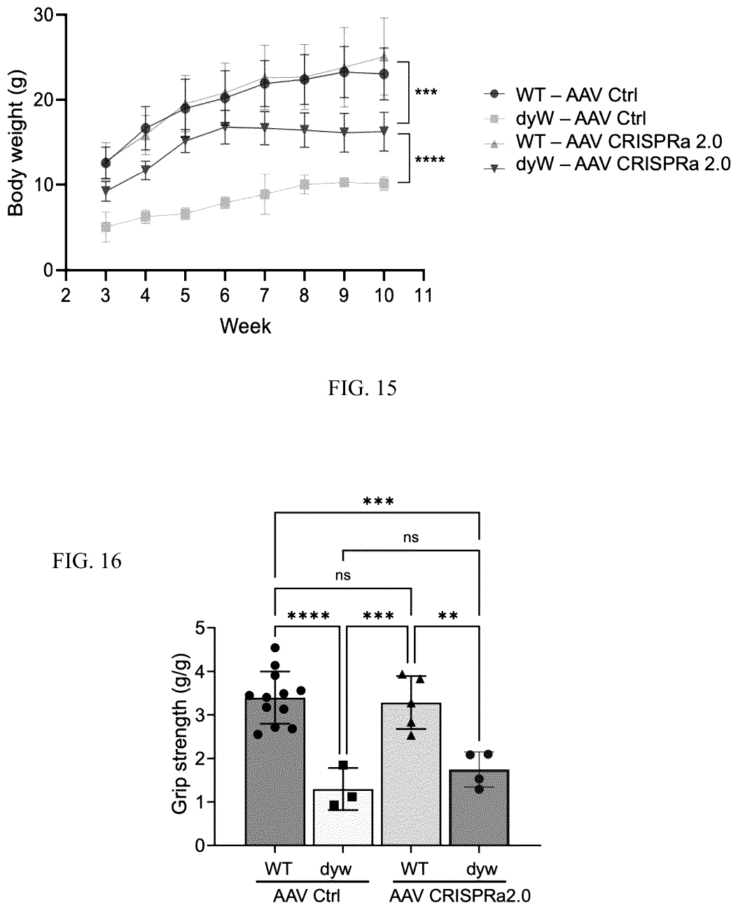

It was shown that disease pathophysiology can be prevented when the mice were treated at neonatal stage. Subsequently, it was examined whether Lama1 upregulation can still be beneficial when administered in older animals. AAV9 CRISPRa 2.0 was injected into 1-week old dy^{w}/dy^{w} mice via retroorbital administration route at the dose of 7.5×10¹¹ vg/animal and monitored their lifespan, body weight, and muscle functions (FIG. 13). It was found that approximately 50% of the dy^{w}/dy^{w} receiving control AAV died by 3 weeks old, with more than 90% of them did not make it past 8 weeks old (FIG. 14). In contrast, all dy^{w}/dy^{w} mice receiving AAV9 CRISPRa 2.0 showed normal life span and were still alive at 28 weeks post-injection. Furthermore, the dy^{w}/dy^{w} mice receiving AAV9 CRISPRa 2.0 showed significant increase in their body weights compared to the control dy^{w}/dy^{W} mice (FIG. 15), indicating the general improvement in the animal's welfare.

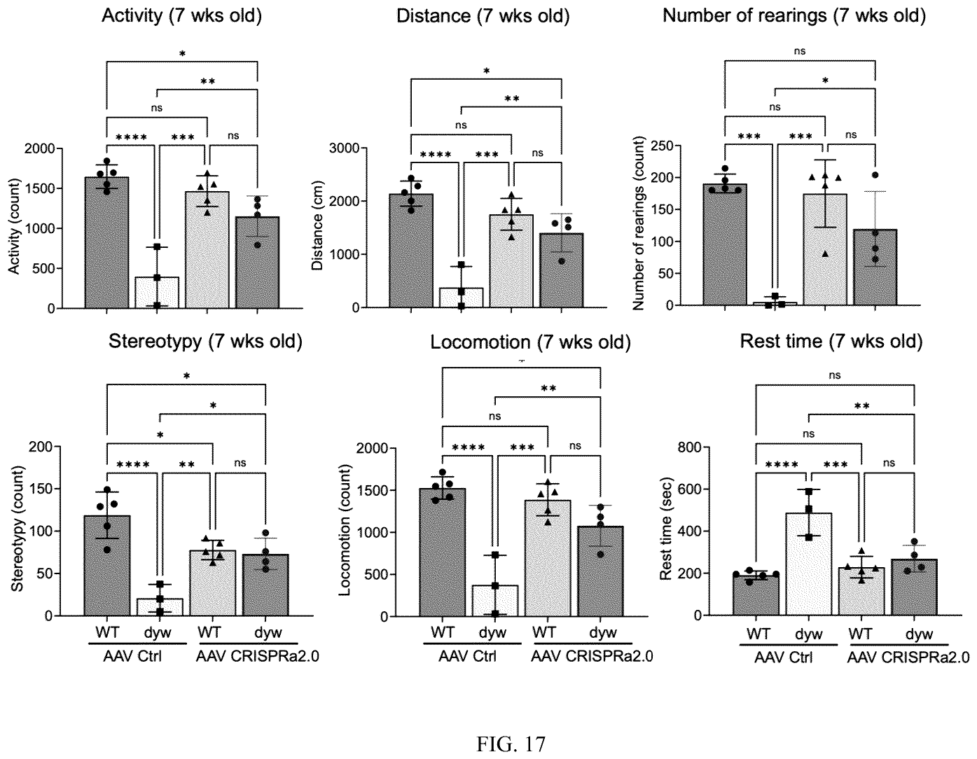

To evaluate the effect of treatment on muscle functions, the animals were tested for grip strength (FIG. 16) and open field (FIG. 17) at the age of 7 weeks old. There was no difference between the dy^{w}/dy^{w} mice receiving AAV control and AAV CRISPRa 2.0 on grip strength (FIG. 16). However, the dy^{w}/dy^{w} mice receiving AAV CRISPRa 2.0 outperformed the control dy^{w}/dy^{w} mice in the open field test, as shown in multiple indicators such as activity, total distance, number of rearing, stereotypy, locomotion, and reduced rest time (FIG. 17). There was no significant difference between the dy^{w}/dy^{w} mice receiving AAV CRISPRa 2.0 to the wildtype counterparts, indicating the remarkable rescue of the phenotypes.

### Materials and Methods

### Viral Vector Injection and Mouse Tissue Harvesting

For in vivo systemic studies, entire litters of P1 newborn mice were injected with CRISPRa 2.0 gRNA1 or no gRNA 7.5E+11 GC intravenously via the temporal vein. Uniformed volume of 30 μL was used, using sterile PBS as a diluent to achieve the desired virus concentration, and delivered via a 31G needle. Newborn mice were cryoanesthetized before injection by placing them directly onto ice for 1 to 2 minutes.

For the delayed treatment study, the mice were genotyped 24 hours before injection. Only wildtype and dy^{w}/dy^{w} mice were used in the study. At the age of 1 week old, the mice were injected via retroorbital route with the same doses of virus as the prevention study. No anesthesia was applied.

Tissues of interest were harvested 5- or 11-weeks post-injection. Tibialis anterior, gastrocnemius, quadriceps, triceps, and heart were snap-frozen in isopentane submerged in liquid nitrogen. Sciatic nerves were placed into optimal cutting temperature compound (OCT, Sakura Finetek) and snap-frozen in isopentane submerged in liquid nitrogen for immunofluorescence analysis. Sciatic nerves for toluidine blue and transmission electron microscopy (TEM) were fixed in cold 2.5% glutaraldehyde and 2% paraformaldehyde in 0.01 M PBS. Brain, liver, kidney, and lung were snap-frozen in liquid nitrogen. Tissues were stored at −80° C. for downstream analysis.

### Muscle Force Assessment

Open field and in situ muscle force assays were carried out on P1 temporal vein systemic injected cohorts. For the open field test, Panlab LE8825 IR actimeter (Harvard Apparatus, USA), which has an infrared tracking system to monitor animal movements was used. The chamber was physically divided into two areas (22.5 cm×22.5 cm each) to allow testing of two mice at the same time. At the start of the test, each mouse was placed in the periphery of the chamber. The system recorded several parameters, including rearing and total distance travelled within the 20 minutes test period. Grip strength was assessed using Gt3 Grip Strength Meter (Bioseb). Mice were grabbed by the tail and lowered forepaws to the metal grip bar and swiftly pulled away. Measurements are averaged from 5 repeated pulls and normalized to body weight. In situ muscle contraction tests were performed using 1 newton 1300A: 3-in-1 Whole Animal System (Aurora Scientific). The mice were anesthetized with 2.5% isoflurane inhalation via SomnoSuite anesthesia system (Kent Scientific). The hindlimb skin of the mouse was carefully removed to expose the calcaneal tendon, in which two double square knots were tied using a 3-0 54 Nylon suture (Teleflex) while leaving a small loop big enough to attach it to the level arm of the 1300A. The tendon alongside a part of the gastrocnemius was then cut to partially detach the muscle from the femur. Contractile output was measured using electrodes that stimulated the nerves to induce the contraction of the gastrocnemius. The maximum tetanic force was measured by stimulating the muscle at 150 Hz, 0.2 pulse width for 0.5 seconds. The specific tetanic force was obtained by correcting to femur length.

### Tissue RNA and Protein Isolation

RNA from the tissues of interest were harvested using TRIzol reagent (Invitrogen) following the manufacturer's instructions. Protein was isolated using RIPA buffer (50 mM Tris HCl pH 7.4, 150 nM NaCl, 1 mM EDTA, 1% deoxycholate, 1% NP40, 1% Triton X-100, and proteinase inhibitor cocktail (Roche)) and homogenized using prefilled 2 mL tubes with 1.4 mm ceramic beads (FisherScientific). cDNA synthesis, RT-PCR, protein quantification, and western blot were carried out.

### Immunofluorescence Staining

Muscles and sciatic nerves were cryo-sectioned at 8 μm thickness and processed according to standard methods. Hematoxylin and eosin staining was performed as per the manufacturer's instruction (Thermo Scientific) and imaged via Revolve microscope (ECHO). Muscle fiber diameter quantification was carried out using imageJ. Feret's diameter distribution of 300 fibers per mouse was recorded and at least three mouse per condition were used for the statistical analysis.

### Toluidine Blue and Transmission Electron Microscopy

Freshly harvested sciatic nerves were fixed in cold 2.5% glutaraldehyde and 2% paraformaldehyde in 0.01 M PBS. Samples were rinsed in PBS, post-fixed in 1% osmium tetroxide with 1% potassium ferricyanide, rinsed in PBS, dehydrated through a graded series of ethanol and propylene oxide, and embedded in Poly/Bed® 812 (Glauert formulations). Semi-thin (300 nm) sections were cut on a Leica Reichart Ultracut, stained with 0.5% Toluidine Blue in 1% sodium borate, and examined under the light microscope. Ultrathin sections (65 nm) were examined on JEOL 1400 Plus transmission electron with a side mount AMT 2k digital camera (Advanced Microscopy Techniques, Danvers, MA).

### Statistical Analysis

The results are presented as means±S.D. unless otherwise indicated. Two experimental groups were compared using Student's t-test. For more than two groups, a two-way analysis of variance (ANOVA) with multiple comparison tests between the groups using Šidák's procedure was used. All procedures were performed using GraphPad Prism 9. Significance is considered for P<0.05.

### Example 3: Exemplary Sequences

Exemplary 4xNRF1 promoter sequence
(SEQ ID NO: 65)
CGCGCATGCGCACCTCTGGGCCGTCGCATGCGCACCAGTCTAGCCGCGCA
GGCGCGCTAGTCAGGCTGCGCATGCGCG
Exemplary 10xNRF1 promoter sequence
(SEQ ID NO: 66)
CGCGCATGCGCACCTCTGGGCCGTCGCATGCGCACCAGTCTAGCCGCGCA
GGCGCGCTAGTCAGGCTGCGCATGCGCGCCATTGCCTGTGCGCAAGCGCG
CTACTCGCACTGCGCATGCGCACCTTTGCATGGGCGCCTGCGCACAATGG
TCTGCGCGCAGGCGCACACGTCAGACTGCGCAGGCGTGCACGTCGGGCGG
CGCATGCGCA
Exemplary dCas9 polypeptide sequence
(SEQ ID NO: 43)
MAPKKKRKVGIHGVPAAKRNYILGLAIGITSVGYGIIDYETRDVIDAGVR
LFKEANVENNEGRRSKRGARRLKRRRRHRIQRVKKLLFDYNLLTDHSELS
GINPYEARVKGLSQKLSEEEFSAALLHLAKRRGVHNVNEVEEDTGNELST
KEQISRNSKALEEKYVAELQLERLKKDGEVRGSINRFKTSDYVKEAKQLL
KVQKAYHQLDQSFIDTYIDLLETRRTYYEGPGEGSPFGWKDIKEWYEMLM
GHCTYFPEELRSVKYAYNADLYNALNDLNNLVITRDENEKLEYYEKFQII
ENVFKQKKKPTLKQIAKEILVNEEDIKGYRVTSTGKPEFTNLKVYHDIKD
ITARKEIIENAELLDQIAKILTIYQSSEDIQEELTNLNSELTQEEIEQIS
NLKGYTGTHNLSLKAINLILDELWHTNDNQIAIFNRLKLVPKKVDLSQQK
EIPTTLVDDFILSPVVKRSFIQSIKVINAIIKKYGLPNDIIIELAREKNS
KDAQKMINEMQKRNRQTNERIEEIIRTTGKENAKYLIEKIKLHDMQEGKC
LYSLEAIPLEDLLNNPFNYEVDHIIPRSVSFDNSFNNKVLVKQEEASKKG
NRTPFQYLSSSDSKISYETFKKHILNLAKGKGRISKTKKEYLLEERDINR
FSVQKDFINRNLVDTRYATRGLMNLLRSYFRVNNLDVKVKSINGGFTSFL
RRKWKFKKERNKGYKHHAEDALIIANADFIFKEWKKLDKAKKVMENQMFE
EKQAESMPEIETEQEYKEIFITPHQIKHIKDFKDYKYSHRVDKKPNRELI
NDTLYSTRKDDKGNTLIVNNLNGLYDKDNDKLKKLINKSPEKLLMYHHDP
QTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVI
KKENYYEVNSKCYEEAKKLKKISNQAEFIASFYNNDLIKINGELYRVIGV
NNDLLNRIEVNMIDITYREYLENMNDKRPPRIIKTIASKTQSIKKYSTDI
LGNLYEVKSKKHPQIIKKGKRPAATKKAGQAKKKK
Nucleic acid sequenceen coding SEQ ID NO: 43
(SEQ ID NO: 78)
ATGGCCCCAAAGAAGAAGCGGAAGGTCGGTATCCACGGAGTCCCAGCAGC
CAAGCGGAACTACATCCTGGGCCTGGCCATCGGCATCACCAGCGTGGGCT
ACGGCATCATCGACTACGAGACACGGGACGTGATCGATGCCGGCGTGCGG
CTGTTCAAAGAGGCCAACGTGGAAAACAACGAGGGCAGGCGGAGCAAGAG
AGGCGCCAGAAGGCTGAAGCGGCGGAGGCGGCATAGAATCCAGAGAGTGA
AGAAGCTGCTGTTCGACTACAACCTGCTGACCGACCACAGCGAGCTGAGC
GGCATCAACCCCTACGAGGCCAGAGTGAAGGGCCTGAGCCAGAAGCTGAG
CGAGGAAGAGTTCTCTGCCGCCCTGCTGCACCTGGCCAAGAGAAGAGGCG
TGCACAACGTGAACGAGGTGGAAGAGGACACCGGCAACGAGCTGTCCACC
AAAGAGCAGATCAGCCGGAACAGCAAGGCCCTGGAAGAGAAATACGTGGC
CGAACTGCAGCTGGAACGGCTGAAGAAAGACGGCGAAGTGCGGGGCAGCA
TCAACAGATTCAAGACCAGCGACTACGTGAAAGAAGCCAAACAGCTGCTG
AAGGTGCAGAAGGCCTACCACCAGCTGGACCAGAGCTTCATCGACACCTA
CATCGACCTGCTGGAAACCCGGCGGACCTACTATGAGGGACCTGGCGAGG
GCAGCCCCTTCGGCTGGAAGGACATCAAAGAATGGTACGAGATGCTGATG
GGCCACTGCACCTACTTCCCCGAGGAACTGCGGAGCGTGAAGTACGCCTA
CAACGCCGACCTGTACAACGCCCTGAACGACCTGAACAATCTCGTGATCA
CCAGGGACGAGAACGAGAAGCTGGAATATTACGAGAAGTTCCAGATCATC
GAGAACGTGTTCAAGCAGAAGAAGAAGCCCACCCTGAAGCAGATCGCCAA
AGAAATCCTCGTGAACGAAGAGGATATTAAGGGCTACAGAGTGACCAGCA
CCGGCAAGCCCGAGTTCACCAACCTGAAGGTGTACCACGACATCAAGGAC
ATTACCGCCCGGAAAGAGATTATTGAGAACGCCGAGCTGCTGGATCAGAT
TGCCAAGATCCTGACCATCTACCAGAGCAGCGAGGACATCCAGGAAGAAC
TGACCAATCTGAACTCCGAGCTGACCCAGGAAGAGATCGAGCAGATCTCT
AATCTGAAGGGCTATACCGGCACCCACAACCTGAGCCTGAAGGCCATCAA
CCTGATCCTGGACGAGCTGTGGCACACCAACGACAACCAGATCGCTATCT
TCAACCGGCTGAAGCTGGTGCCCAAGAAGGTGGACCTGTCCCAGCAGAAA
GAGATCCCCACCACCCTGGTGGACGACTTCATCCTGAGCCCCGTCGTGAA
GAGAAGCTTCATCCAGAGCATCAAAGTGATCAACGCCATCATCAAGAAGT
ACGGCCTGCCCAACGACATCATTATCGAGCTGGCCCGCGAGAAGAACTCC
AAGGACGCCCAGAAAATGATCAACGAGATGCAGAAGCGGAACCGGCAGAC
CAACGAGCGGATCGAGGAAATCATCCGGACCACCGGCAAAGAGAACGCCA
AGTACCTGATCGAGAAGATCAAGCTGCACGACATGCAGGAAGGCAAGTGC
CTGTACAGCCTGGAAGCCATCCCTCTGGAAGATCTGCTGAACAACCCCTT
CAACTATGAGGTGGACCACATCATCCCCAGAAGCGTGTCCTTCGACAACA
GCTTCAACAACAAGGTGCTCGTGAAGCAGGAAGAAGCCAGCAAGAAGGGC
AACCGGACCCCATTCCAGTACCTGAGCAGCAGCGACAGCAAGATCAGCTA
CGAAACCTTCAAGAAGCACATCCTGAATCTGGCCAAGGGCAAGGGCAGAA
TCAGCAAGACCAAGAAAGAGTATCTGCTGGAAGAACGGGACATCAACAGG
TTCTCCGTGCAGAAAGACTTCATCAACCGGAACCTGGTGGATACCAGATA
CGCCACCAGAGGCCTGATGAACCTGCTGCGGAGCTACTTCAGAGTGAACA
ACCTGGACGTGAAAGTGAAGTCCATCAATGGCGGCTTCACCAGCTTTCTG
CGGCGGAAGTGGAAGTTTAAGAAAGAGCGGAACAAGGGGTACAAGCACCA
CGCCGAGGACGCCCTGATCATTGCCAACGCCGATTTCATCTTCAAAGAGT
GGAAGAAACTGGACAAGGCCAAAAAAGTGATGGAAAACCAGATGTTCGAG
GAAAAGCAGGCCGAGAGCATGCCCGAGATCGAAACCGAGCAGGAGTACAA
AGAGATCTTCATCACCCCCCACCAGATCAAGCACATTAAGGACTTCAAGG
ACTACAAGTACAGCCACCGGGTGGACAAGAAGCCTAATAGAGAGCTGATT
AACGACACCCTGTACTCCACCCGGAAGGACGACAAGGGCAACACCCTGAT
CGTGAACAATCTGAACGGCCTGTACGACAAGGACAATGACAAGCTGAAAA
AGCTGATCAACAAGAGCCCCGAAAAGCTGCTGATGTACCACCACGACCCC
CAGACCTACCAGAAACTGAAGCTGATTATGGAACAGTACGGCGACGAGAA
GAATCCCCTGTACAAGTACTACGAGGAAACCGGGAACTACCTGACCAAGT
ACTCCAAAAAGGACAACGGCCCCGTGATCAAGAAGATTAAGTATTACGGC
AACAAACTGAACGCCCATCTGGACATCACCGACGACTACCCCAACAGCAG
AAACAAGGTCGTGAAGCTGTCCCTGAAGCCCTACAGATTCGACGTGTACC
TGGACAATGGCGTGTACAAGTTCGTGACCGTGAAGAATCTGGATGTGATC
AAAAAAGAAAACTACTACGAAGTGAATAGCAAGTGCTATGAGGAAGCTAA
GAAGCTGAAGAAGATCAGCAACCAGGCCGAGTTTATCGCCTCCTTCTACA
ACAACGATCTGATCAAGATCAACGGCGAGCTGTATAGAGTGATCGGCGTG
AACAACGACCTGCTGAACCGGATCGAAGTGAACATGATCGACATCACCTA
CCGCGAGTACCTGGAAAACATGAACGACAAGAGGCCCCCCAGGATCATTA
AGACAATCGCCTCCAAGACCCAGAGCATTAAGAAGTACAGCACAGACATT
CTGGGCAACCTGTATGAAGTGAAATCTAAGAAGCACCCTCAGATCATCAA
AAAGGGCAAAAGGCCGGCGGCCACGAAAAAGGCCGGCCAGGCAAAAAAGA
AAAAG
Exemplary VP64-Δp65-ΔRTA sequence
(SEQ ID NO: 44)
DALDDEDLDMLGSDALDDFDLDMLGSDALDDEDLDMLGSDALDDEDLDML
INSRSSGSPKKKRKVGSGGGSGGSGSVLPQAPAPAPAPAMVSALAQAPAP
VPVLAPGPPQAVAPPAPKPTQAGEGTLSEALLQLQFDDEDLGALLGNSTD
PAVFTDLASVDNSEFQQLLNQGIPVAPHTTEPMLMEYPEAITRLVTGAQR
PPDPAPAPLGAPGLPNGLLSGDEDFSSIADMDFSALLSGGGSGGSGSDLS
HPPPRGHLDELTTTLESMTEDLNLDSPLTPELNEILDTFLNDECLLHAMH
ISTGLSIFDTSLF
thefirstbold/underlinedsection = VP64
thesecondbold/underlinedsection = Δp65
thethirdbold/underlinedsection = ΔRTA
Nucleic acid sequence encoding SEQ ID NO: 44
(SEQ ID NO: 79)
GACGCATTGGACGATTTTGATCTGGATATGCTGGGAAGTGACGCCCTCGA
TGATTTTGACCTTGACATGCTTGGTTCGGATGCCCTTGATGACTTTGACC
TCGACATGCTCGGCAGTGACGCCCTTGATGATTTCGACCTGGACATGCTG
ATTAACTCTAGAAGTTCCGGATCTCCGAAAAAGAAACGCAAAGTTGGTTC
GGGAGGTGGTTCGGGTGGCTCTGGATCAGTGCTGCCTCAGGCTCCTGCTC
CTGCACCAGCTCCAGCCATGGTGTCTGCACTGGCTCAGGCACCAGCACCC
GTGCCTGTGCTGGCTCCTGGACCTCCACAGGCTGTGGCTCCACCAGCCCC
TAAACCTACACAGGCCGGCGAGGGCACACTGTCTGAAGCTCTGCTGCAGC
TGCAGTTCGACGACGAGGATCTGGGAGCCCTGCTGGGAAACAGCACCGAT
CCTGCCGTGTTCACCGACCTGGCCAGCGTGGACAACAGCGAGTTCCAGCA
GCTGCTGAACCAGGGCATCCCTGTGGCCCCTCACACCACCGAGCCCATGC
TGATGGAATACCCCGAGGCCATCACCCGGCTCGTGACAGGCGCTCAGAGG
CCTCCTGATCCAGCTCCTGCCCCTCTGGGAGCACCAGGCCTGCCTAATGG
ACTGCTGTCTGGCGACGAGGACTTCAGCTCTATCGCCGATATGGATTTCT
CAGCCTTGCTGTCAGGCGGTGGTAGTGGTGGGAGCGGTAGTGACCTTTCC
CATCCGCCCCCAAGGGGCCATCTGGATGAGCTGACAACCACACTTGAGTC
CATGACCGAGGATCTGAACCTGGACTCACCCCTGACCCCGGAATTGAACG
AGATTCTGGATACCTTCCTGAACGACGAGTGCCTCTTGCATGCCATGCAT
ATCAGCACAGGACTGTCCATCTTCGACACATCTCTGTTT

the first bold/underlined section=DNA sequence encoding VP64
the second bold/underlined section=DNA sequence encoding Δp65
the third bold/underlined section=DNA sequence encoding ΔRTA

### Example 4: Exemplary Synthetic Construct

This Example shows a nucleic acid sequence of an exemplary double stranded engineered DNA sequence comprising (1) first ITR, (2) a U6 promoter operably linked to a DNA sequence encoding a gRNA that is complementary to a target site upstream of a transcriptional start site of an endogenous nucleic acid sequence encoding a Lama1 polypeptide (a Lama1 gRNA), (3) a 4×NRF1 promoter operably linked to a DNA sequence encoding a SadCas9 polypeptide and a VP64-Δp65-ΔRTA transcriptional activator, and (4) a second ITR. These components are annotated as shown below.

Total length 5029 bp, with 325 bp of buffer bp between the different components
first section of bold text: AAV ITR 2×141 bp
first section of bold/underlined text: U6 promoter+Lama1 2RNA1+Sa gRNA scaffold 350 bp
second section of bold text: 4×NRF1 78 bp
second section of bold/underlined text: SadCas9 3255 bp
third section of bold text: VP64 150 bp
third section of bold/underlined text: delP65 357 bp
fourth section of bold text: delRTA 198 bp
fourth section of bold/underlined text: 2×sNRP-1 terminator 34 bp
fifth section of bold text: AAV ITR 2×141 bp

(SEQ ID NO: 80)
cctgcaggcagctgcgcgctcgctcgctcactgaggccgcccgggcaaagcccgggcgtcgggcgacctttggtcgcccggc
ctcagtgagcgagcgagcgcgcagaggacgtccgtcgacgcgcgagcgagcgagtgactccggcgggcccgtttcgggcccg
cagcccgctggaaaccaggggccggagtcactcgctcgctcgcgcgtctgagggagtggccaactccatcactaggggttcc
tgcggccgcctcgaggagggcctatttcccatgattccttcatatttgcatatacgatacaaggctgttagagactccctca
ccggttgaggtagtgatccccaaggacgccggcggagctcctcccggataaagggtactaaggaagtataaacgtatatgct
atgttccgacaatctctgataattggaattaatttgactgtaaacacaaagatattagtacaaaatacgtgacgtagaaagt
aataatttcttgggtagtttgcagttttaaaattatgttttactattaaccttaattaaactgacatttgtgtttctataat
catgttttatgcactgcatctttcattattaaagaacccatcaaacgtcaaaattttaatacaaaataaatggactatcata
tgcttaccgtaacttgaaagtatttcgatttcttggctttatatatcttgtggaaaggacgaaaCACCGAAGGGCCTGCGCA
CCAGGCgttttttacctgatagtatacgaatggcattgaactttcataaagctaaagaaccgaaatatatagaacacctttc
ctgctttGTGGCTTCCCGGACGCGTGGTCCGcaaatagtactctggaaacagaatctactaaaacaaggcaaaatgccgtgt
ttatctcgtcaacttgttggcgagaTTTTTggcgttCGCGCATGCGCACCTCTGGGCCGTatcatgagacctttgtcttaga
tgattttgttccgttttacggcacaaatagagcagttgaacaaccgctctAAAAAccgcaaGCGCGTACGCGTGGAGACCCG
GCACGCATGCGCACCAGTCTAGCCGCGCAGGCGCGCTAGTCAGGCTGCGCATGCGCGctctggctaactaccggtgccacca
tggccccaaagaagaagcggaaggtcggGCGTACGCGTGGTCAGATCGGCGCGTCCGCGCGATCAGTCCGACGCGTACGCGC
gagaccgattgatggccacggtggtaccggggtttcttcttcgccttccagcctatccacggagtcccagcagccaagcgga
actacatcctgggcctggccatcggcatcaccagcgtgggctacggcatcatcgactacgagacacgggacgtgatcgatag
gtgcctcagggtcgtcggttcgccttgatgtaggacccggaccggtagccgtagtggtcgcacccgatgccgtagtagctga
tgctctgtgccctgcactagcatgccggcgtgcggctgttcaaagaggccaacgtggaaaacaacgagggcaggcggagcaa
gagaggcgccagaaggctgaagcggcggaggcggcatagaatccagtacggccgcacgccgacaagtttctccggttgcacc
ttttgttgctcccgtccgcctcgttctctccgcggtcttccgacttcgccgcctccgccgtatcttaggtcagagtgaagaa
gctgctgttcgactacaacctgctgaccgaccacagcgagctgagcggcatcaacccctacgaggccagagtgaagggcctg
agccagaagctgagtctcacttcttcgacgacaagctgatgttggacgactggctggtgtcgctcgactcgccgtagttggg
gatgctccggtctcacttcccggactcggtcttcgactccgaggaagagttctctgccgccctgctgcacctggccaagaga
agaggcgtgcacaacgtgaacgaggtggaagaggacaccggcaacgagctgtccaccaaagagcgctccttctcaagagacg
gcgggacgacgtggaccggttctcttctccgcacgtgttgcacttgctccaccttctcctgtggccgttgctcgacaggtgg
tttctcgagatcagccggaacagcaaggccctggaagagaaatacgtggccgaactgcagctggaacggctgaagaaagacg
gcgaagtgcggggcagcatcaacagattcaagtctagtcggccttgtcgttccgggaccttctctttatgcaccggcttgac
gtcgaccttgccgacttctttctgccgcttcacgccccgtcgtagttgtctaagttcaccagcgactacgtgaaagaagcca
aacagctgctgaaggtgcagaaggcctaccaccagctggaccagagcttcatcgacacctacatcgacctgctggaaacccg
tggtcgctgatgcactttcttcggtttgtcgacgacttccacgtcttccggatggtggtcgacctggtctcgaagtagctgt
ggatgtagctggacgacctttgggcgcggacctactatgagggacctggcgagggcagccccttcggctggaaggacatcaa
agaatggtacgagatgctgatgggccactgcacctacttccccgaggaaccgcctggatgatactccctggaccgctcccgt
cggggaagccgaccttcctgtagtttcttaccatgctctacgactacccggtgacgtggatgaaggggctccttgtgcggag
cgtgaagtacgcctacaacgccgacctgtacaacgccctgaacgacctgaacaatctcgtgatcaccagggacgagaacgag
aagctggaatattacgagacgcctcgcacttcatgcggatgttgcggctggacatgttgcgggacttgctggacttgttaga
gcactagtggtccctgctcttgctcttcgaccttataatgctcaagttccagatcatcgagaacgtgttcaagcagaagaag
aagcccaccctgaagcagatcgccaaagaaatcctcgtgaacgaagaggatattaagggctacagagtttcaaggtctagta
gctcttgcacaagttcgtcttcttcttcggggggacttcgtctagcggtttctttaggagcacttgcttctcctataattcc
cgatgtctcagaccagcaccggcaagcccgagttcaccaacctgaaggtgtaccacgacatcaaggacattaccgcccggaa
agagattattgagaacgccgagctgctggatcagactggtcgtggccgttcgggctcaagtggttggacttccacatggtgc
tgtagttcctgtaatgggggcctttctctaataactcttgcggctcgacgacctagtctttgccaagatcctgaccatctac
cagagcagcgaggacatccaggaagaactgaccaatctgaactccgagctgacccaggaagagatcgagcagatctctaatc
tgaacggttctaggactggtagatggtctcgtcgctcctgtaggtccttcttgactggttagacttgaggctcgactgggtc
cttctctagctcgtctagagattagacaagggctataccggcacccacaacctgagcctgaaggccatcaacctgatcctgg
acgagctgtggcacaccaacgacaaccagatcgctatcttcaaccggctgaattcccgatatggccgtgggtgttggactcg
gacttccggtagttggactaggacctgctcgacaccgtgtggttgctgttggtctagcgatagaagttggccgacttgctgg
tgcccaagaaggtggacctgtcccagcagaaagagatccccaccaccctggtggacgacttcatcctgagccccgtcgtgaa
gagaagcttcatccagagcacgaccacgggttcttccacctggacagggtcgtctttctctaggggtggtgggaccacctgc
tgaagtaggactcggggcagcacttctcttcgaagtaggtctcgttcaaagtgatcaacgccatcatcaagaagtacggcct
gcccaacgacatcattatcgagctggcccgcgagaagaactccaaggacgcccagaaaatgatcaacgagagtttcactagt
tgcggtagtagttcttcatgccggacgggttgctgtagtaatagctcgaccgggcgctcttcttgaggttcctgcgggtctt
ttactagttgctcatgcagaagcggaaccggcagaccaacgagcggatcgaggaaatcatccggaccaccggcaaagagaac
gccaagtacctgatcgagaagatcaagctgcacgacattacgtcttcgccttggccgtctggttgctcgcctagctccttta
gtaggcctggtggccgtttctcttgcggttcatggactagctcttctagttcgacgtgctgtagcaggaaggcaagtgcctg
tacagcctggaagccatccctctggaagatctgctgaacaaccccttcaactatgaggtggaccacatcatccccagaagcg
tgtcctcgtccttccgttcacggacatgtcggaccttcggtagggagaccttctagacgacttgttggggaagttgatactc
cacctggtgtagtaggggtcttcgcacaggatcgacaacagcttcaacaacaaggtgctcgtgaagcaggaagaagccagca
agaagggcaaccggaccccattccagtacctgagcagcagcgacagcaagatcagcagctgttgtcgaagttgttgttccac
gagcacttcgtccttcttcggtcgttcttcccgttggcctggggtaaggtcatggactcgtcgtcgctgtcgttctagtcgt
acgaaaccttcaagaagcacatcctgaatctggccaagggcaagggcagaatcagcaagaccaagaaagagtatctgctgga
agaacgggacatcaacaggttctcatgctttggaagttcttcgtgtaggacttagaccggttcccgttcccgtcttagtcgt
tctggttctttctcatagacgaccttcttgccctgtagttgtccaagagcgtgcagaaagacttcatcaaccggaacctggt
ggataccagatacgccaccagaggcctgatgaacctgctgcggagctacttcagagtgaacaacctggacgtgagcacgtct
ttctgaagtagttggccttggaccacctatggtctatgcggtggtctccggactacttggacgacgcctcgatgaagtctca
cttgttggacctgcactaagtgaagtccatcaatggcggcttcaccagctttctgcggcggaagtggaagtttaagaaagag
cggaacaaggggtacaagcaccacgccgaggacgccctgatcttcacttcaggtagttaccgccgaagtggtcgaaagacgc
cgccttcaccttcaaattctttctcgccttgttccccatgttcgtggtgcggctcctgcgggactagattgccaacgccgat
ttcatcttcaaagagtggaagaaactggacaaggccaaaaaagtgatggaaaaccagatgttcgaggaaaagcaggccgaga
gcatgcccgataacggttgcggctaaagtagaagtttctcaccttctttgacctgttccggttttttcactaccttttggtc
tacaagctccttttcgtccggctctcgtacgggctgatcgaaaccgagcaggagtacaaagagatcttcatcaccccccacc
agatcaagcacattaaggacttcaaggactacaagtacagccaccgggtggacaagaagcctagctttggctcgtcctcatg
tttctctagaagtagtggggggggtctagttcgtgtaattcctgaagttcctgatgttcatgtcggtggcccacctgttctt
cgctaatagagagctgattaacgacaccctgtactccacccggaaggacgacaagggcaacaccctgatcgtgaacaatctg
aacggcctgtacgacaaggacaatgacgattatctctcgactaattgctgtgggacatgaggtgggccttcctgctgttccc
gttgtgggactagcacttgttagacttgccggacatgctgttcctgttactgaagctgaaaaagctgatcaacaagagcccc
gaaaagctgctgatgtaccaccacgacccccagacctaccagaaactgaagctgattatggaacagtacggcgacgattcga
ctttttcgactagttgttctcggggcttttcgacgactacatggtggtgctgggggtctggatggtctttgacttcgactaa
taccttgtcatgccgctgctgaagaatcccctgtacaagtactacgaggaaaccgggaactacctgaccaagtactccaaaa
aggacaacggccccgtgatcaagaagattaagtattacggcaacacttcttaggggacatgttcatgatgctcctttggccc
ttgatggactggttcatgaggtttttcctgttgccggggcactagttcttctaattcataatgccgttgtaactgaacgccc
atctggacatcaccgacgactaccccaacagcagaaacaaggtcgtgaagctgtccctgaagccctacagattcgacgtgta
cctggacaatggcttgacttgcgggtagacctgtagtggctgctgatggggttgtcgtctttgttccagcacttcgacaggg
acttcgggatgtctaagctgcacatggacctgttaccggtgtacaagttcgtgaccgtgaagaatctggatgtgatcaaaaa
agaaaactactacgaagtgaatagcaagtgctatgaggaagctaagaagctgaagaagatcagcacatgttcaagcactggc
acttcttagacctacactagttttttcttttgatgatgcttcacttatcgttcacgatactccttcgattcttcgacttctt
ctagtccaaccaggccgagtttatcgcctccttctacaacaacgatctgatcaagatcaacggcgagctgtatagagtgatc
ggcgtgaacaacgacctgctgaaccggatcggttggtccggctcaaatagcggaggaagatgttgttgctagactagttcta
gttgccgctcgacatatctcactagccgcacttgttgctggacgacttggcctagcaagtgaacatgatcgacatcacctac
cgcgagtacctggaaaacatgaacgacaagaggccccccaggatcattaagacaatcgcctccaagacccagagcattaagt
tcacttgtactagctgtagtggatggcgctcatggaccttttgtacttgctgttctccggggggtcctagtaattctgttag
cggaggttctgggtctcgtaattcaagtacagcacagacattctgggcaacctgtatgaagtgaaatctaagaagcaccctc
agatcatcaaaaagggcaaaaggccggcggccacgaaaaaggccggccattcatgtcgtgtctgtaagacccgttggacata
cttcactttagattcttcgtgggagtctagtagtttttcccgttttccggccgccggtgctttttccggccggtggcaaaaa
agaaaaagggatccgaggccagcggttccggacgggctgacgcattggacgattttgatctggatatgctgggaagtgacgc
cctcgatgattttgaccccgttttttctttttccctaggctccggtcgccaaggcctgcccgactgcgtaacctgctaaaac
tagacctatacgacccttcactgcgggagctactaaaactggttgacatgcttggttcggatgcccttgatgactttgacct
cgacatgctcggcagtgacgcccttgatgatttcgacctggacatgctgattaactctagaagttccaactgtacgaaccaa
gcctacgggaactactgaaactggagctgtacgagccgtcactgcgggaactactaaagctggacctgtacgactaattgag
atcttcaaggggatctccgaaaaagaaacgcaaagttggttcgggaggtggttcgggtggctctggatcagtgctgcctcag
gctcctgctcctgcaccagctccagccatggtgtccctagaggctttttctttgcgtttcaaccaagccctccaccaagccc
accgagacctagtcacgacggagtccgaggacgaggacgtggtcgaggtcggtaccacagtgcactggctcaggcaccagca
cccgtgcctgtgctggctcctggacctccacaggctgtggctccaccagcccctaaacctacacaggccggcgagggcacac
tgtacgtgaccgagtccgtggtcgtgggcacggacacgaccgaggacctggaggtgtccgacaccgaggtggtcggggattt
ggatgtgtccggccgctcccgtgtgacactgaagctctgctgcagctgcagttcgacgacgaggatctgggagccctgctgg
gaaacagcaccgatcctgccgtgttcaccgacctggccagcgtggacaacagcgacttcgagacgacgtcgacgtcaagctg
ctgctcctagaccctcgggacgaccctttgtcgtggctaggacggcacaagtggctggaccggtcgcacctgttgtcggagt
tccagcagctgctgaaccagggcatccctgtggcccctcacaccaccgagcccatgctgatggaataccccgaggccatcac
ccggctcgtgacaggcgctcactcaaggtcgtcgacgacttggtcccgtagggacaccggggagtgtggtggctcgggtacg
actaccttatggggctccggtagtgggccgagcactgtccgcgagtgaggcctcctgatccagctcctgcccctctgggagc
accaggcctgcctaatggactgctgtctggcgacgaggacttcagctctatcgccgatatggatttctcagctccggaggac
taggtcgaggacggggagaccctcgtggtccggacggattacctgacgacagaccgctgctcctgaagtcgagatagcggct
atacctaaagagtcccttgctgtcaggcggtggtagtggtgggagcggtagtgacctttcccatccgcccccaaggggccat
ctggatgagctgacaaccacacttgagtccatgaccgagggaacgacagtccgccaccatcaccaccctcgccatcactgga
aagggtaggcgggggttccccggtagacctactcgactgttggtgtgaactcaggtactggctcgatctgaacctggactca
cccctgaccccggaattgaacgagattctggataccttcctgaacgacgagtgcctcttgcatgccatgcatatcagcacag
gactgtcctagacttggacctgagtggggactggggccttaacttgctctaagacctatggaaggacttgctgctcacggag
aacgtacggtacgtatagtcgtgtcctgacagcatcttcgacacatctctgttttaggaattcaaataaaatacgaaatgaa
ataaaatacgaaatgcaattgggccgcaggaacccctagtgatggagttggccactcgtagaagctgtgtagagacaaaatc
cttaagtttattttatgctttactttattttatgctttacgttaacccggcgtccttggggatcactacctcaaccggtgag
cctctctgcgcgctcgctcgctcactgaggccgggcgaccaaaggttgcccgacgcccgggctttgcccgggcggcctcagt
gagcgagcgagcgcgcagctgcctgggagagacgcgcgagcgagcgagtgactccggcccgctggtttccaacgggctgcgg
gcccgaaacgggcccgccggagtcactcgctcgctcgcgcgtcgacggaccagggtcc

### Example 5: Treating LAMA2-RD

A human identified as having LAMA2-RD is administered a composition including a population of a single AAV vector designed for targeted gene activation of nucleic acid encoding a Lama1 polypeptide (e.g., designed to have a genome including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide, and (b) a nucleic acid encoding a dCas polypeptide including one or more transcriptional activators). The single AAV vector designed for targeted gene activation of nucleic acid encoding a Lama1 polypeptide is effective to increase expression of a Lama1 polypeptide by cells within that mammal.

### Example 6: Treating LAMA2-RD

A human identified as having LAMA2-RD is administered a composition including a population of a single AAV vector designed for targeted gene activation of nucleic acid encoding a Lama1 polypeptide (e.g., designed to have a genome including a nucleic acid sequence (e.g., an engineered DNA sequence) including (a) a nucleic acid encoding a gRNA that is complementary to a target site upstream of a transcriptional start site of an endogenous nucleic acid sequence encoding a Lama1 polypeptide, and (b) a nucleic acid encoding a dCas polypeptide including one or more transcriptional activators). The single AAV vector designed for targeted gene activation of nucleic acid encoding a Lama1 polypeptide is effective to reduce one or more symptoms of LAMA2-RD within that mammal.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An adeno-associated virus comprising a single-stranded DNA, wherein said single-stranded DNA comprises a first inverted terminal repeat (ITR) sequence followed by an engineered DNA sequence followed by a second ITR sequence, wherein said engineered DNA sequence comprises (a) a first DNA sequence comprising a first promotor sequence operably linked to a DNA sequence encoding a guide RNA or (a-ii) a reverse-complement thereof and (b) a second DNA sequence comprising (b-i) a second promotor sequence operably linked to a DNA sequence encoding a catalytically inactive Cas polypeptide, wherein said guide RNA is at least 95 percent complementary to a genomic sequence located between 0 and 1000 nucleotides upstream of a transcriptional start site of an endogenous nucleic acid sequence encoding a Lama1 polypeptide within a mammal, wherein said second promotor sequence comprises at least four motifs having the DNA sequence set forth in SEQ ID NO:45 (5′-YGCGCANGCGCR-3′), wherein 8 to 12 intervening nucleotides are located between each adjacent motif of said at least four motifs, wherein said catalytically inactive Cas polypeptide comprises at least one transcriptional activator, and wherein delivery of said adeno-associated virus to a cell within said mammal results in expression of said Lama1 polypeptide within said cell.

2. The adeno-associated virus of claim 1, said mammal is a human.

3. The adeno-associated virus of claim 1, wherein said cell is a muscle cell.

4. The adeno-associated virus of claim 1, wherein said cell is a skeletal muscle cell.

5. The adeno-associated virus of claim 1, wherein said cell is a Schwann cell.

6. The adeno-associated virus of claim 1, wherein said adeno-associated virus is AAV6, AAV8, AAV2, or AAV9.

7. The adeno-associated virus of claim 1, wherein said first ITR sequence is directly followed by said engineered DNA sequence.

8. The adeno-associated virus of claim 1, wherein said first ITR sequence is indirectly followed by said engineered DNA sequence.

9. The adeno-associated virus of claim 1, wherein said engineered DNA sequence is directly followed by said second ITR sequence.

10. The adeno-associated virus of claim 1, wherein said engineered DNA sequence is indirectly followed by said second ITR sequence.

11. The adeno-associated virus of claim 1, wherein said first ITR sequence comprises the DNA sequence set forth in SEQ ID NO:67.

12. The adeno-associated virus of claim 1, wherein said second ITR sequence comprises the DNA sequence set forth in SEQ ID NO:69.

13. The adeno-associated virus of claim 1, wherein said first promotor sequence is a type 3 RNA polymerase III promoter.

14. The adeno-associated virus of claim 13, wherein said first promotor sequence is a 7SK promoter, a H1 promoter, or a U6 promotor.

15. The adeno-associated virus of claim 1, wherein said guide RNA is complementary to said genomic sequence.

16. The adeno-associated virus of claim 1, wherein said genomic sequence is located between 0 and 500 nucleotides upstream of said transcriptional start site.

17. The adeno-associated virus of claim 1, wherein said guide RNA comprises the RNA sequence set forth in SEQ ID NO:1.

18. The adeno-associated virus of claim 1, wherein said second promotor sequence comprises four of said motifs.

19. The adeno-associated virus of claim 1, wherein each of said motifs comprise a DNA sequence set forth in any one of SEQ ID NOs: 46 to 55.

20. The adeno-associated virus of claim 1, wherein each of said motifs comprise a DNA sequence set forth in any one of SEQ ID NOs: 46 to 49.

21. The adeno-associated virus of claim 1, wherein each of said 8 to 12 intervening nucleotides comprise a DNA sequence set forth in any one of SEQ ID NOs: 56 to 64.

22. The adeno-associated virus of claim 1, wherein each of said 8 to 12 intervening nucleotides comprise a DNA sequence set forth in any one of SEQ ID NOs: 56 to 58.

23. The adeno-associated virus of claim 1, wherein said catalytically inactive Cas polypeptide is a catalytically inactive Cas9 polypeptide.

24. The adeno-associated virus of claim 1, wherein said catalytically inactive Cas polypeptide is a Staphylococcus aureus deactivated Cas9 (SadCas9) polypeptide.

25. The adeno-associated virus of claim 1, wherein said catalytically inactive Cas polypeptide comprises the amino acid sequence set forth in SEQ ID NO:43.

26. The adeno-associated virus of claim 1, wherein said catalytically inactive Cas polypeptide comprises a VP64 transcriptional activator.

27. The adeno-associated virus of claim 1, wherein said catalytically inactive Cas polypeptide comprises a VP64-Δp65-ΔRTA transcriptional activator.

28. The adeno-associated virus of claim 27, wherein said VP64-Δp65-ΔRTA transcriptional activator comprises the amino acid sequence set forth in SEQ ID NO:44.

29. An isolated nucleic acid molecule comprising the engineered DNA sequence of claim 1.

30. An in vitro host cell, wherein said host cell comprises an adeno-associated virus of claim 1.

31. An in vitro host cell, wherein said host cell comprises a nucleic acid of claim 29.

32. The host cell of claim 30, wherein said host cell is a muscle cell, a fibroblast, a Schwann cell, or an epithelial cell.

33. A composition comprising an adeno-associated virus of claim 1.

34. A method for increasing expression of a Lama1 polypeptide by cells within a mammal, wherein said method comprises administering an adeno-associated virus of claim 1 to said mammal, thereby increasing expression of said Lama1 polypeptide by said cells within said mammal.

35. The method of claim 34, wherein said mammal is a human.

36. A method for treating muscular dystrophy, wherein said method comprises administering an adeno-associated virus of claim 1 to a mammal having muscular dystrophy, wherein expression of a Lama1 polypeptide by cells within said mammal is increased following said administering step, and wherein severity of a symptom of said muscular dystrophy is reduced following said administering step.

37. The method of claim 36, wherein said mammal is a human.


SEQUENCE LISTING (84 sequences)

SEQ ID NO: 1 - RNA, 20 nt, synthetic construct
  source 1..20: mol_type = other RNA
gaagggcctgcgcaccaggc

SEQ ID NO: 2 - RNA, 21 nt, synthetic construct
  source 1..21: mol_type = other RNA
gcggggcgcggccaggcaggc

SEQ ID NO: 3 - RNA, 22 nt, synthetic construct
  source 1..22: mol_type = other RNA
gaccccaactggagtggagggt

SEQ ID NO: 4 - RNA, 22 nt, synthetic construct
  source 1..22: mol_type = other RNA
gcacggcggggctccccctgga

SEQ ID NO: 5 - RNA, 20 nt, synthetic construct
  source 1..20: mol_type = other RNA
ggcggggcggggcgcagccg

SEQ ID NO: 6 - RNA, 21 nt, synthetic construct
  source 1..21: mol_type = other RNA
caagctgggcgcccccggggg

SEQ ID NO: 7 - RNA, 21 nt, synthetic construct
  source 1..21: mol_type = other RNA
aggccaagctgggcgcccccg

SEQ ID NO: 8 - RNA, 21 nt, synthetic construct
  source 1..21: mol_type = other RNA
gtcagcccggcctccccgact

SEQ ID NO: 9 - RNA, 21 nt, synthetic construct
  source 1..21: mol_type = other RNA
agccggggaggcggccgcggt

SEQ ID NO: 10 - RNA, 21 nt, synthetic construct
  source 1..21: mol_type = other RNA
gcagcccagtttctcctcccc

SEQ ID NO: 11 - RNA, 21 nt, synthetic construct
  source 1..21: mol_type = other RNA
ccggacccccgcagcccagtt

SEQ ID NO: 12 - RNA, 21 nt, synthetic construct
  source 1..21: mol_type = other RNA
cctcccggacccccgcagccc

SEQ ID NO: 13 - RNA, 21 nt, synthetic construct
  source 1..21: mol_type = other RNA
tcagctgggcacatagtttct

SEQ ID NO: 14 - RNA, 21 nt, synthetic construct
  source 1..21: mol_type = other RNA
gcagggccgcggcggggcggg

SEQ ID NO: 15 - RNA, 21 nt, synthetic construct
  source 1..21: mol_type = other RNA
cccagctcctggcaggggcgc

SEQ ID NO: 16 - RNA, 21 nt, synthetic construct
  source 1..21: mol_type = other RNA
ccgagcctgggtggcttcccg

SEQ ID NO: 17 - RNA, 21 nt, synthetic construct
  source 1..21: mol_type = other RNA
agggcggcgggtcggccgagc

SEQ ID NO: 18 - RNA, 21 nt, synthetic construct
  source 1..21: mol_type = other RNA
ctgcccacggcggaaagggcg

SEQ ID NO: 19 - RNA, 21 nt, synthetic construct
  source 1..21: mol_type = other RNA
aagtcctctgcccacggcgga

SEQ ID NO: 20 - RNA, 21 nt, synthetic construct
  source 1..21: mol_type = other RNA
tttcagaaaaaatcctcaagt

SEQ ID NO: 21 - RNA, 21 nt, synthetic construct
  source 1..21: mol_type = other RNA
gcgtcctctctctccagcagt

SEQ ID NO: 22 - DNA, 20 bp, synthetic construct
  source 1..20: mol_type = other DNA
gaagggcctgcgcaccaggc

SEQ ID NO: 23 - DNA, 21 bp, synthetic construct
  source 1..21: mol_type = other DNA
gcggggcgcggccaggcaggc

SEQ ID NO: 24 - DNA, 22 bp, synthetic construct
  source 1..22: mol_type = other DNA
gaccccaactggagtggagggt

SEQ ID NO: 25 - DNA, 22 bp, synthetic construct
  source 1..22: mol_type = other DNA
gcacggcggggctccccctgga

SEQ ID NO: 26 - DNA, 20 bp, synthetic construct
  source 1..20: mol_type = other DNA
ggcggggcggggcgcagccg

SEQ ID NO: 27 - DNA, 21 bp, synthetic construct
  source 1..21: mol_type = other DNA
caagctgggcgcccccggggg

SEQ ID NO: 28 - DNA, 21 bp, synthetic construct
  source 1..21: mol_type = other DNA
aggccaagctgggcgcccccg

SEQ ID NO: 29 - DNA, 21 bp, synthetic construct
  source 1..21: mol_type = other DNA
gtcagcccggcctccccgact

SEQ ID NO: 30 - DNA, 21 bp, synthetic construct
  source 1..21: mol_type = other DNA
agccggggaggcggccgcggt

SEQ ID NO: 31 - DNA, 21 bp, synthetic construct
  source 1..21: mol_type = other DNA
gcagcccagtttctcctcccc

SEQ ID NO: 32 - DNA, 21 bp, synthetic construct
  source 1..21: mol_type = other DNA
ccggacccccgcagcccagtt

SEQ ID NO: 33 - DNA, 21 bp, synthetic construct
  source 1..21: mol_type = other DNA
cctcccggacccccgcagccc

SEQ ID NO: 34 - DNA, 21 bp, synthetic construct
  source 1..21: mol_type = other DNA
tcagctgggcacatagtttct

SEQ ID NO: 35 - DNA, 21 bp, synthetic construct
  source 1..21: mol_type = other DNA
gcagggccgcggcggggcggg

SEQ ID NO: 36 - DNA, 21 bp, synthetic construct
  source 1..21: mol_type = other DNA
cccagctcctggcaggggcgc

SEQ ID NO: 37 - DNA, 21 bp, synthetic construct
  source 1..21: mol_type = other DNA
ccgagcctgggtggcttcccg

SEQ ID NO: 38 - DNA, 21 bp, synthetic construct
  source 1..21: mol_type = other DNA
agggcggcgggtcggccgagc

SEQ ID NO: 39 - DNA, 21 bp, synthetic construct
  source 1..21: mol_type = other DNA
ctgcccacggcggaaagggcg

SEQ ID NO: 40 - DNA, 21 bp, synthetic construct
  source 1..21: mol_type = other DNA
aagtcctctgcccacggcgga

SEQ ID NO: 41 - DNA, 21 bp, synthetic construct
  source 1..21: mol_type = other DNA
tttcagaaaaaatcctcaagt

SEQ ID NO: 42 - DNA, 21 bp, synthetic construct
  source 1..21: mol_type = other DNA
gcgtcctctctctccagcagt

SEQ ID NO: 43 - protein, 1085 aa, synthetic construct
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 44 - protein, 313 aa, synthetic construct
DALDDFDLDMLGSDALDDFDLDMLGSDALDDFDLDMLGSDALDDFDLDMLINSRSSGSPKKKRKVGSGGGSGGSGSVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSGGGSGGSGSDLSHPPPRGHLDELTTTLESMTEDLNLDSPLTPELNEILDTFLNDECLLHAMHISTGLSIFDTSLF

SEQ ID NO: 45 - DNA, 12 bp, synthetic construct
  source 1..12: mol_type = other DNA
ygcgcangcgcr

SEQ ID NO: 46 - DNA, 12 bp, synthetic construct
  source 1..12: mol_type = other DNA
cgcgcatgcgca

SEQ ID NO: 47 - DNA, 12 bp, synthetic construct
  source 1..12: mol_type = other DNA
gtcgcatgcgca

SEQ ID NO: 48 - DNA, 12 bp, synthetic construct
  source 1..12: mol_type = other DNA
cgcgcaggcgcg

SEQ ID NO: 49 - DNA, 12 bp, synthetic construct
  source 1..12: mol_type = other DNA
tgcgcatgcgcg

SEQ ID NO: 50 - DNA, 12 bp, synthetic construct
  source 1..12: mol_type = other DNA
tgcgcaagcgcg

SEQ ID NO: 51 - DNA, 12 bp, synthetic construct
  source 1..12: mol_type = other DNA
tgcgcatgcgca

SEQ ID NO: 52 - DNA, 12 bp, synthetic construct
  source 1..12: mol_type = other DNA
ggcgcctgcgca

SEQ ID NO: 53 - DNA, 12 bp, synthetic construct
  source 1..12: mol_type = other DNA
cgcgcaggcgca

SEQ ID NO: 54 - DNA, 12 bp, synthetic construct
  source 1..12: mol_type = other DNA
tgcgcaggcgtg

SEQ ID NO: 55 - DNA, 12 bp, synthetic construct
  source 1..12: mol_type = other DNA
ggcgcatgcgca

SEQ ID NO: 56 - DNA, 10 bp, synthetic construct
  source 1..10: mol_type = other DNA
cctctgggcc

SEQ ID NO: 57 - DNA, 10 bp, synthetic construct
  source 1..10: mol_type = other DNA
ccagtctagc

SEQ ID NO: 58 - DNA, 10 bp, synthetic construct
  source 1..10: mol_type = other DNA
ctagtcaggc

SEQ ID NO: 59 - DNA, 10 bp, synthetic construct
  source 1..10: mol_type = other DNA
ccattgcctg

SEQ ID NO: 60 - DNA, 10 bp, synthetic construct
  source 1..10: mol_type = other DNA
ctactcgcac

SEQ ID NO: 61 - DNA, 10 bp, synthetic construct
  source 1..10: mol_type = other DNA
cctttgcatg

SEQ ID NO: 62 - DNA, 10 bp, synthetic construct
  source 1..10: mol_type = other DNA
caatggtctg

SEQ ID NO: 63 - DNA, 10 bp, synthetic construct
  source 1..10: mol_type = other DNA
cacgtcagac

SEQ ID NO: 64 - DNA, 10 bp, synthetic construct
  source 1..10: mol_type = other DNA
cacgtcgggc

SEQ ID NO: 65 - DNA, 78 bp, synthetic construct
  source 1..78: mol_type = other DNA
cgcgcatgcgcacctctgggccgtcgcatgcgcaccagtctagccgcgcaggcgcgctagtcaggctgcgcatgcgcg

SEQ ID NO: 66 - DNA, 210 bp, synthetic construct
  source 1..210: mol_type = other DNA
cgcgcatgcgcacctctgggccgtcgcatgcgcaccagtctagccgcgcaggcgcgctagtcaggctgcgcatgcgcgccattgcctgtgcgcaagcgcgctactcgcactgcgcatgcgcacctttgcatgggcgcctgcgcacaatggtctgcgcgcaggcgcacacgtcagactgcgcaggcgtgcacgtcgggcggcgcatgcgca

SEQ ID NO: 67 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 68 - DNA, 141 bp, synthetic construct
  source 1..141: mol_type = other DNA
ggacgtccgtcgacgcgcgagcgagcgagtgactccggcgggcccgtttcgggcccgcagcccgctggaaaccagcgggccggagtcactcgctcgctcgcgcgtctctccctcaccggttgaggtagtgatccccaagga

SEQ ID NO: 69 - DNA, 141 bp, synthetic construct
  source 1..141: mol_type = other DNA
aggaacccctagtgatggagttggccactccctctctgcgcgctcgctcgctcactgaggccgggcgaccaaaggttgcccgacgcccgggctttgcccgggcggcctcagtgagcgagcgagcgcgcagctgcctgcagg

SEQ ID NO: 70 - DNA, 141 bp, synthetic construct
  source 1..141: mol_type = other DNA
tccttggggatcactacctcaaccggtgagggagagacgcgcgagcgagcgagtgactccggcccgctggtttccaacgggctgcgggcccgaaacgggcccgccggagtcactcgctcgctcgcgcgtcgacggacgtcc

SEQ ID NO: 71 - DNA, 20 bp, synthetic construct
  source 1..20: mol_type = other DNA
gtcccctccaccccacagtg

SEQ ID NO: 72 - DNA, 23 bp, synthetic construct
  source 1..23: mol_type = other DNA
ggaaggttacaaagttcgattgg

SEQ ID NO: 73 - DNA, 23 bp, synthetic construct
  source 1..23: mol_type = other DNA
acgtgaaataagaccttgccatc

SEQ ID NO: 74 - DNA, 20 bp, synthetic construct
  source 1..20: mol_type = other DNA
agcatgcccgagatcgaaac

SEQ ID NO: 75 - DNA, 20 bp, synthetic construct
  source 1..20: mol_type = other DNA
tgttgcccttgtcgtccttc

SEQ ID NO: 76 - DNA, 21 bp, synthetic construct
  source 1..21: mol_type = other DNA
tgtttgtgatgggtgtgaacc

SEQ ID NO: 77 - DNA, 20 bp, synthetic construct
  source 1..20: mol_type = other DNA
actgtggtcatgagcccttc

SEQ ID NO: 78 - DNA, 3255 bp, synthetic construct
  source 1..3255: mol_type = other DNA
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 79 - DNA, 939 bp, synthetic construct
  source 1..939: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggttcgggaggtggttcgggtggctctggatcagtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgtcaggcggtggtagtggtgggagcggtagtgacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 80 - DNA, 10066 bp, synthetic construct
  source 1..10066: mol_type = other DNA
cctgcaggcagctgcgcgctcgctcgctcactgaggccgcccgggcaaagcccgggcgtcgggcgacctttggtcgcccggcctcagtgagcgagcgagcgcgcagaggacgtccgtcgacgcgcgagcgagcgagtgactccggcgggcccgtttcgggcccgcagcccgctggaaaccagcgggccggagtcactcgctcgctcgcgcgtctgagggagtggccaactccatcactaggggttcctgcggccgcctcgaggagggcctatttcccatgattccttcatatttgcatatacgatacaaggctgttagagactccctcaccggttgaggtagtgatccccaaggacgccggcggagctcctcccggataaagggtactaaggaagtataaacgtatatgctatgttccgacaatctctgataattggaattaatttgactgtaaacacaaagatattagtacaaaatacgtgacgtagaaagtaataatttcttgggtagtttgcagttttaaaattatgttttactattaaccttaattaaactgacatttgtgtttctataatcatgttttatgcactgcatctttcattattaaagaacccatcaaacgtcaaaattttaatacaaaataaatggactatcatatgcttaccgtaacttgaaagtatttcgatttcttggctttatatatcttgtggaaaggacgaaacaccgaagggcctgcgcaccaggcgttttttacctgatagtatacgaatggcattgaactttcataaagctaaagaaccgaaatatatagaacacctttcctgctttgtggcttcccggacgcgtggtccgcaaatagtactctggaaacagaatctactaaaacaaggcaaaatgccgtgtttatctcgtcaacttgttggcgagatttttggcgttcgcgcatgcgcacctctgggccgtatcatgagacctttgtcttagatgattttgttccgttttacggcacaaatagagcagttgaacaaccgctctaaaaaccgcaagcgcgtacgcgtggagacccggcacgcatgcgcaccagtctagccgcgcaggcgcgctagtcaggctgcgcatgcgcgctctggctaactaccggtgccaccatggccccaaagaagaagcggaaggtcgggcgtacgcgtggtcagatcggcgcgtccgcgcgatcagtccgacgcgtacgcgcgagaccgattgatggccacggtggtaccggggtttcttcttcgccttccagcctatccacggagtcccagcagccaagcggaactacatcctgggcctggccatcggcatcaccagcgtgggctacggcatcatcgactacgagacacgggacgtgatcgataggtgcctcagggtcgtcggttcgccttgatgtaggacccggaccggtagccgtagtggtcgcacccgatgccgtagtagctgatgctctgtgccctgcactagcatgccggcgtgcggctgttcaaagaggccaacgtggaaaacaacgagggcaggcggagcaagagaggcgccagaaggctgaagcggcggaggcggcatagaatccagtacggccgcacgccgacaagtttctccggttgcaccttttgttgctcccgtccgcctcgttctctccgcggtcttccgacttcgccgcctccgccgtatcttaggtcagagtgaagaagctgctgttcgactacaacctgctgaccgaccacagcgagctgagcggcatcaacccctacgaggccagagtgaagggcctgagccagaagctgagtctcacttcttcgacgacaagctgatgttggacgactggctggtgtcgctcgactcgccgtagttggggatgctccggtctcacttcccggactcggtcttcgactccgaggaagagttctctgccgccctgctgcacctggccaagagaagaggcgtgcacaacgtgaacgaggtggaagaggacaccggcaacgagctgtccaccaaagagcgctccttctcaagagacggcgggacgacgtggaccggttctcttctccgcacgtgttgcacttgctccaccttctcctgtggccgttgctcgacaggtggtttctcgagatcagccggaacagcaaggccctggaagagaaatacgtggccgaactgcagctggaacggctgaagaaagacggcgaagtgcggggcagcatcaacagattcaagtctagtcggccttgtcgttccgggaccttctctttatgcaccggcttgacgtcgaccttgccgacttctttctgccgcttcacgccccgtcgtagttgtctaagttcaccagcgactacgtgaaagaagccaaacagctgctgaaggtgcagaaggcctaccaccagctggaccagagcttcatcgacacctacatcgacctgctggaaacccgtggtcgctgatgcactttcttcggtttgtcgacgacttccacgtcttccggatggtggtcgacctggtctcgaagtagctgtggatgtagctggacgacctttgggcgcggacctactatgagggacctggcgagggcagccccttcggctggaaggacatcaaagaatggtacgagatgctgatgggccactgcacctacttccccgaggaaccgcctggatgatactccctggaccgctcccgtcggggaagccgaccttcctgtagtttcttaccatgctctacgactacccggtgacgtggatgaaggggctccttgtgcggagcgtgaagtacgcctacaacgccgacctgtacaacgccctgaacgacctgaacaatctcgtgatcaccagggacgagaacgagaagctggaatattacgagacgcctcgcacttcatgcggatgttgcggctggacatgttgcgggacttgctggacttgttagagcactagtggtccctgctcttgctcttcgaccttataatgctcaagttccagatcatcgagaacgtgttcaagcagaagaagaagcccaccctgaagcagatcgccaaagaaatcctcgtgaacgaagaggatattaagggctacagagtttcaaggtctagtagctcttgcacaagttcgtcttcttcttcgggtgggacttcgtctagcggtttctttaggagcacttgcttctcctataattcccgatgtctcagaccagcaccggcaagcccgagttcaccaacctgaaggtgtaccacgacatcaaggacattaccgcccggaaagagattattgagaacgccgagctgctggatcagactggtcgtggccgttcgggctcaagtggttggacttccacatggtgctgtagttcctgtaatggcgggcctttctctaataactcttgcggctcgacgacctagtctttgccaagatcctgaccatctaccagagcagcgaggacatccaggaagaactgaccaatctgaactccgagctgacccaggaagagatcgagcagatctctaatctgaacggttctaggactggtagatggtctcgtcgctcctgtaggtccttcttgactggttagacttgaggctcgactgggtccttctctagctcgtctagagattagacaagggctataccggcacccacaacctgagcctgaaggccatcaacctgatcctggacgagctgtggcacaccaacgacaaccagatcgctatcttcaaccggctgaattcccgatatggccgtgggtgttggactcggacttccggtagttggactaggacctgctcgacaccgtgtggttgctgttggtctagcgatagaagttggccgacttgctggtgcccaagaaggtggacctgtcccagcagaaagagatccccaccaccctggtggacgacttcatcctgagccccgtcgtgaagagaagcttcatccagagcacgaccacgggttcttccacctggacagggtcgtctttctctaggggtggtgggaccacctgctgaagtaggactcggggcagcacttctcttcgaagtaggtctcgttcaaagtgatcaacgccatcatcaagaagtacggcctgcccaacgacatcattatcgagctggcccgcgagaagaactccaaggacgcccagaaaatgatcaacgagagtttcactagttgcggtagtagttcttcatgccggacgggttgctgtagtaatagctcgaccgggcgctcttcttgaggttcctgcgggtcttttactagttgctcatgcagaagcggaaccggcagaccaacgagcggatcgaggaaatcatccggaccaccggcaaagagaacgccaagtacctgatcgagaagatcaagctgcacgacattacgtcttcgccttggccgtctggttgctcgcctagctcctttagtaggcctggtggccgtttctcttgcggttcatggactagctcttctagttcgacgtgctgtagcaggaaggcaagtgcctgtacagcctggaagccatccctctggaagatctgctgaacaaccccttcaactatgaggtggaccacatcatccccagaagcgtgtcctcgtccttccgttcacggacatgtcggaccttcggtagggagaccttctagacgacttgttggggaagttgatactccacctggtgtagtaggggtcttcgcacaggatcgacaacagcttcaacaacaaggtgctcgtgaagcaggaagaagccagcaagaagggcaaccggaccccattccagtacctgagcagcagcgacagcaagatcagcagctgttgtcgaagttgttgttccacgagcacttcgtccttcttcggtcgttcttcccgttggcctggggtaaggtcatggactcgtcgtcgctgtcgttctagtcgtacgaaaccttcaagaagcacatcctgaatctggccaagggcaagggcagaatcagcaagaccaagaaagagtatctgctggaagaacgggacatcaacaggttctcatgctttggaagttcttcgtgtaggacttagaccggttcccgttcccgtcttagtcgttctggttctttctcatagacgaccttcttgccctgtagttgtccaagagcgtgcagaaagacttcatcaaccggaacctggtggataccagatacgccaccagaggcctgatgaacctgctgcggagctacttcagagtgaacaacctggacgtgagcacgtctttctgaagtagttggccttggaccacctatggtctatgcggtggtctccggactacttggacgacgcctcgatgaagtctcacttgttggacctgcactaagtgaagtccatcaatggcggcttcaccagctttctgcggcggaagtggaagtttaagaaagagcggaacaaggggtacaagcaccacgccgaggacgccctgatcttcacttcaggtagttaccgccgaagtggtcgaaagacgccgccttcaccttcaaattctttctcgccttgttccccatgttcgtggtgcggctcctgcgggactagattgccaacgccgatttcatcttcaaagagtggaagaaactggacaaggccaaaaaagtgatggaaaaccagatgttcgaggaaaagcaggccgagagcatgcccgataacggttgcggctaaagtagaagtttctcaccttctttgacctgttccggttttttcactaccttttggtctacaagctccttttcgtccggctctcgtacgggctgatcgaaaccgagcaggagtacaaagagatcttcatcaccccccaccagatcaagcacattaaggacttcaaggactacaagtacagccaccgggtggacaagaagcctagctttggctcgtcctcatgtttctctagaagtagtggggggtggtctagttcgtgtaattcctgaagttcctgatgttcatgtcggtggcccacctgttcttcgctaatagagagctgattaacgacaccctgtactccacccggaaggacgacaagggcaacaccctgatcgtgaacaatctgaacggcctgtacgacaaggacaatgacgattatctctcgactaattgctgtgggacatgaggtgggccttcctgctgttcccgttgtgggactagcacttgttagacttgccggacatgctgttcctgttactgaagctgaaaaagctgatcaacaagagccccgaaaagctgctgatgtaccaccacgacccccagacctaccagaaactgaagctgattatggaacagtacggcgacgattcgactttttcgactagttgttctcggggcttttcgacgactacatggtggtgctgggggtctggatggtctttgacttcgactaataccttgtcatgccgctgctgaagaatcccctgtacaagtactacgaggaaaccgggaactacctgaccaagtactccaaaaaggacaacggccccgtgatcaagaagattaagtattacggcaacacttcttaggggacatgttcatgatgctcctttggcccttgatggactggttcatgaggtttttcctgttgccggggcactagttcttctaattcataatgccgttgtaactgaacgcccatctggacatcaccgacgactaccccaacagcagaaacaaggtcgtgaagctgtccctgaagccctacagattcgacgtgtacctggacaatggcttgacttgcgggtagacctgtagtggctgctgatggggttgtcgtctttgttccagcacttcgacagggacttcgggatgtctaagctgcacatggacctgttaccggtgtacaagttcgtgaccgtgaagaatctggatgtgatcaaaaaagaaaactactacgaagtgaatagcaagtgctatgaggaagctaagaagctgaagaagatcagcacatgttcaagcactggcacttcttagacctacactagttttttcttttgatgatgcttcacttatcgttcacgatactccttcgattcttcgacttcttctagtccaaccaggccgagtttatcgcctccttctacaacaacgatctgatcaagatcaacggcgagctgtatagagtgatcggcgtgaacaacgacctgctgaaccggatcggttggtccggctcaaatagcggaggaagatgttgttgctagactagttctagttgccgctcgacatatctcactagccgcacttgttgctggacgacttggcctagcaagtgaacatgatcgacatcacctaccgcgagtacctggaaaacatgaacgacaagaggccccccaggatcattaagacaatcgcctccaagacccagagcattaagttcacttgtactagctgtagtggatggcgctcatggaccttttgtacttgctgttctccggggggtcctagtaattctgttagcggaggttctgggtctcgtaattcaagtacagcacagacattctgggcaacctgtatgaagtgaaatctaagaagcaccctcagatcatcaaaaagggcaaaaggccggcggccacgaaaaaggccggccattcatgtcgtgtctgtaagacccgttggacatacttcactttagattcttcgtgggagtctagtagtttttcccgttttccggccgccggtgctttttccggccggtggcaaaaaagaaaaagggatccgaggccagcggttccggacgggctgacgcattggacgattttgatctggatatgctgggaagtgacgccctcgatgattttgaccccgttttttctttttccctaggctccggtcgccaaggcctgcccgactgcgtaacctgctaaaactagacctatacgacccttcactgcgggagctactaaaactggttgacatgcttggttcggatgcccttgatgactttgacctcgacatgctcggcagtgacgcccttgatgatttcgacctggacatgctgattaactctagaagttccaactgtacgaaccaagcctacgggaactactgaaactggagctgtacgagccgtcactgcgggaactactaaagctggacctgtacgactaattgagatcttcaaggggatctccgaaaaagaaacgcaaagttggttcgggaggtggttcgggtggctctggatcagtgctgcctcaggctcctgctcctgcaccagctccagccatggtgtccctagaggctttttctttgcgtttcaaccaagccctccaccaagcccaccgagacctagtcacgacggagtccgaggacgaggacgtggtcgaggtcggtaccacagtgcactggctcaggcaccagcacccgtgcctgtgctggctcctggacctccacaggctgtggctccaccagcccctaaacctacacaggccggcgagggcacactgtacgtgaccgagtccgtggtcgtgggcacggacacgaccgaggacctggaggtgtccgacaccgaggtggtcggggatttggatgtgtccggccgctcccgtgtgacactgaagctctgctgcagctgcagttcgacgacgaggatctgggagccctgctgggaaacagcaccgatcctgccgtgttcaccgacctggccagcgtggacaacagcgacttcgagacgacgtcgacgtcaagctgctgctcctagaccctcgggacgaccctttgtcgtggctaggacggcacaagtggctggaccggtcgcacctgttgtcggagttccagcagctgctgaaccagggcatccctgtggcccctcacaccaccgagcccatgctgatggaataccccgaggccatcacccggctcgtgacaggcgctcactcaaggtcgtcgacgacttggtcccgtagggacaccggggagtgtggtggctcgggtacgactaccttatggggctccggtagtgggccgagcactgtccgcgagtgaggcctcctgatccagctcctgcccctctgggagcaccaggcctgcctaatggactgctgtctggcgacgaggacttcagctctatcgccgatatggatttctcagctccggaggactaggtcgaggacggggagaccctcgtggtccggacggattacctgacgacagaccgctgctcctgaagtcgagatagcggctatacctaaagagtcccttgctgtcaggcggtggtagtggtgggagcggtagtgacctttcccatccgcccccaaggggccatctggatgagctgacaaccacacttgagtccatgaccgagggaacgacagtccgccaccatcaccaccctcgccatcactggaaagggtaggcgggggttccccggtagacctactcgactgttggtgtgaactcaggtactggctcgatctgaacctggactcacccctgaccccggaattgaacgagattctggataccttcctgaacgacgagtgcctcttgcatgccatgcatatcagcacaggactgtcctagacttggacctgagtggggactggggccttaacttgctctaagacctatggaaggacttgctgctcacggagaacgtacggtacgtatagtcgtgtcctgacagcatcttcgacacatctctgttttaggaattcaaataaaatacgaaatgaaataaaatacgaaatgcaattgggccgcaggaacccctagtgatggagttggccactcgtagaagctgtgtagagacaaaatccttaagtttattttatgctttactttattttatgctttacgttaacccggcgtccttggggatcactacctcaaccggtgagcctctctgcgcgctcgctcgctcactgaggccgggcgaccaaaggttgcccgacgcccgggctttgcccgggcggcctcagtgagcgagcgagcgcgcagctgcctgggagagacgcgcgagcgagcgagtgactccggcccgctggtttccaacgggctgcgggcccgaaacgggcccgccggagtcactcgctcgctcgcgcgtcgacggaccagggtcc

SEQ ID NO: 81 - DNA, 340 bp, synthetic construct
  source 1..340: mol_type = other DNA
aatgctcaccccaactggagtggagggtcagagtaccacccaggagaggcaccccgccaccaccaccaggggcagaacagatccccttgggaagagacccctaggaaggactccgcacggcggggctccccctggaggacagacctgggcgcagcggggcgcctttgggggaggtcctgggtaggacaggggcggggcgcggccaggcaggcgggggtgtgtccgaagggcctgcgcaccaggccagggtcgagctgggaagagcacactcgcgcagtgcctgtgagcgagcacgggcgagaccttccccaggagcgcagggagcggcggcgacaacatg

SEQ ID NO: 82 - DNA, 2051 bp, synthetic construct
  source 1..2051: mol_type = other DNA
actagtgtctggtccaagcagtggagtgcggaggctctcagaaaaggcagccgttttattaaactcatttttcaccctgagcaggtctccacactctccgcagccctggaggcaatttaggtatttgccaagcagagtgaggagtggaaaactctgtcctgtgaactttcataaaacatttgggatgaagatattttgaccattccattttatccgtgtgctgttgtgaaattttcttttgctgaaacagtcctgcttgattctgagccgcgattgtaagtcatgacctgtggctcctgatgcttccgatctgcgatctgcgatctgcaggtgatggctgttctcctgcccgtaactccagcaccaggtgtgactgacctgcaggctgtatttggggagctgtctggaccttggagccctcttacattgagtcgtatggtgacagtacaggaaactgggtgagaaatcagatctgagctctagccccacactaaacacaagcaggtaagacaggcctgagcttacttcctggtctctcccaggagcctctctaactgcccctcttagagaaaatcacagacatgaatgaatgttctttacgatgccctatgcatgtatctaatatataagcaaggcagttatttagatgcaaaaacatttgtgatgttctaaatgtttaacgcttgagtaattatttttagatgcttactttacccctcatcactcacttgccatctaggtattcattttaatatttaagtagttaagaacatctgactttaacctttagtgatgtgttgagtggcattactgagccacagtggattttttccccgtgtgtgtgataatttcgcctgtgctaattacccaccacatgcatactcagtgaacgcagaggtcaagagagggtgttgtatccttggaactagagttacagaagcttgtgagcctgaattgagccctgatttttgcaagaacagcaagtgttttaagcccctaaaccctttttgcagccccccccccccgtacctgtttaaagggccaaacggtacaggtttgttttgtacttcctgttaacaaataatagccaactattctcattagaatttactcattacgtggttaaggacctgtgcttagagaaaccgggaattggtagagttttcaaagtgatttagacaaactgaacaaaaatgacctcctcctatgaaagagaaaacggcacagggcatcgtgggaaacgtcatggtgtctgtgcatagtgtggaaatgcgttatgaatacgtttgcgaggaaactaaactaaaactttctcaggtttaaaacgagtttttccaaaatgagcgttgagacttggagactacccttatacttaagtcccagcaaccgtctgagctttccctaggcaaaaaagctacccattttctgaagggggctggcaaacaaacaaaatgaaaacacagccacccaaaccaaaaccactgagagtgtcctgtgcaggaacagacaggatatagggtcccgcaagaatggactgtgacctgactatatttctacagctggggattggccaagactgcacagttccaacaggcagaaaagggtcaacagggcaaatactttcttggaatccaccctccagggccaggcttaacagaaacctgcttctccccggcccctgtctctccaccttcctccctattggaccctgaatgctcaccccaactggagtggagggtcagagtaccacccaggagaggcaccccgccaccaccaccaggggcagaacagatccccttgggaagagacccctaggaaggactccgcacggcggggctccccctggaggacagacctgggcgcagcggggcgcctttgggggaggtcctgggtaggacaggggcggggcgcggccaggcaggcgggggtgtgtccgaagggcctgcgcaccaggccagggtcgagctgggaagagcacactcgcgcagtgcctgtgagcgagcacgggcgagaccttccccaggagcgcagggagcggcggcgacaacatg

SEQ ID NO: 83 - DNA, 2170 bp, synthetic construct
  source 1..2170: mol_type = other DNA
attcttttcattgaaatcttttctgcttaattttgagcagtgataaatagtgccaatttgtaacttttgatgcttcctattcactccccagaggtgctgaccacttgcctcccttgaaattacacacgtgagatccctgcttgcagttcaaagataaggtcccattgacctcctgcctgaatttctaggaacccaggatatggggagacccagaatatggggacaccccttgtgattcctccagtgtggttttttttttttttttttttgagacggagtcttgctctgtcgcccaggcgggggtgcagtggcgctatcctggctcactgcaagctccgcctcccgggttcacaccattctcctgtctcagcctcccgagtagctgggactacaggcgcctgccaccgccccccggctaattttttgtatttttagtggagacggggtttcaccgcgttagccaggatggtctcgatctcctgacctcaggtgatccacccgcctcggcctcccaaagtgctgggattacaggcgttagccaccacctccaagtttgcttttgatcaacatcctctgtgatttcagcgtggtgttgtaaaggactatggactagaaaccagacatggggtctagctctagaccagacatttgctgacactgagaactcacttccttatctcctggcaagcatctcatagagttgtgaagatcccaaaagataatctatatgaatgctaggtctgtgtgtgcagttatttcagtgcataaatgcttataattctgcattttaatagctatgtgcctaatactataatatttgatcatttattaaatactaagtttacttatttaataattacttagtaacattttaatagttacgaacgtctgcattttaaatttagtactgtatggagtattcactgaactatattgcatccttttaaaacggccaaactatcattttgttccatgatttttaaaaattaatcgatggctaagtactataactaagttctactcactagatgtgtaaggaagtgtcctcagaaaaacaggaattgatagagttgtcaaatagattcacacaagtgagaggaaattatcttcctatcaagaaaaaaaaaccctgcaaaataagatggtgggatgataatgagatattgtccctgtatatttatattgagaaaatgtgaaatctacaaagtgttatgaataccttttttttcttttttgagacagagtctcgctctgtcgcccaggctggagtgcagctggtgtgatctcagctcactgcaacctcccactcccgggttcaagcaattctcctgcctcagcctcccaagtagctgggactgcaagcgcgtgccaccacacccagctaatttttgtatgtttttagtagagacgaggtttcaccatgatggccaggctggtttcgaactcctgacctcaagtgatccacccgcctcggcctcccaaagtgctggaattacaggcgtgagccactgcacccatccaatacttttgatagaaaaaaaaaatcagaggaaattaaaaattctttatcggggaccagacatagttttaaaataataataattataataacagcagttgaagttgcgtgtcaggagcgcgtcctctctctccagcagtaagggtatctaattataataattaagtccaagtgaccttgccttctttaggcaaacaaagtcgggaacaagttgcaactcattttcagaaaaaatcctcaagtcctctgcccacggcggaaagggcggcgggtcggccgagcctgggtggcttcccgaggggtcgcctctcgcccagctcctggcaggggcgcgtgggtcctggcccggggactgcagggccgcggcggggcgggcagggtcagctgggcacatagtttctcggaatccaccctcccggacccccgcagcccagtttctcctcccctcagcccaccttctctgcccacctcctacccggagccggggaggcggccgcggtcagcccggcctccccgacttggggtccgcgtgcgccgggacccagaggccaagctgggcgcccccgggggaggggtctgggtggcggggccaagccggggcgggacggggcggggcggggcgcagccgaggggtgtgtccgagggcggctg

SEQ ID NO: 84 - DNA, 599 bp, synthetic construct
  source 1..599: mol_type = other DNA
ttgaagttgcgtgtcaggagcgcgtcctctctctccagcagtaagggtatctaattataataattaagtccaagtgaccttgaattctttaggcaaacaaagtcgggaacaagttgcaactcattttcagaaaaaatcctcaagtcctctgcccacggcggaaagggcggcgggtcggccgagcctgggtggattcccgaggggtcgcctctcgcccagctcctggcaggggcgcgtgggtcctggcccggggactgcagggccgcggcggggcgggcagggtcagctgggcacatagtttctcggaatccaccctcccggacccccgcagcccagttctcctcccctcagcccaccttctctgcccacctcctacccggagccggggaggcggccgcggtcagcccggcctccccgacttggggtccgcgtgcgccgggacccagaggccaagctgggcgcccccgggggaggggtctgggtggcggggccaagccggggcgggacggggcggggcggggcgcagccgaggggtgtgtccgagggcggctggagggaggggcggcaggctccaggagcggcggggccagggcagcgcg